(19) United States
(12) Patent Application Publication
(10) Pub. No.: US 2024/0376472 A1
(43) Pub. Date: Nov. 14, 2024

(54) Conditional Double Stranded Antisense Oligonucleotides

(71) Applicant: Emory University, Atlanta, GA (US)

(72) Inventors: Jiahui Zhang, Dedham, MA (US); Khalid Salaita, Atlanta, GA (US); Hanjoong Jo, Atlanta, GA (US)

(21) Appl. No.: 18/683,467

(22) PCT Filed: Aug. 17, 2022
(86) PCT No.: PCT/US2022/075049

§ 371 Date: Feb. 13, 2024

Related U.S. Application Data
(60) Provisional application No. 63/233,979, filed on Aug. 17, 2021.

(51) Int. Cl. C12N 15/113

(52) U.S. Cl. CPC C12N 15/113; C12N 2310/113; C12N 2310/315; C12N 2310/3231

(57) ABSTRACT

This disclosure relates to conditional double stranded antisense oligonucleotides and uses in managing diseases and conditions. In certain embodiments, the conditional double stranded antisense oligonucleotides are non-naturally occurring double stranded nucleobase polymer complexes comprising a first strand and a second strand, wherein the first strand is an antisense oligonucleotide connected to a segment that is partially identical to cell specific RNA, e.g., microRNA, and the second strand is a locking strand configured to release the first strand when in the presence of the cells specific RNA; thus, releasing the antisense oligonucleotide in a manner limited to cells that express a specific RNA.

### CROSS-REFERENCE TO RELATED APPLICATIONS

This application claims the benefit of U.S. Provisional Application No. 63/233,979 filed Aug. 17, 2021. The entirety of this application is hereby incorporated by reference for all purposes.

### STATEMENT REGARDING FEDERALLY SPONSORED RESEARCH OR DEVELOPMENT

This invention was made with government support under HL142866, HL095070, and HL139757 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21033PCT.xml. The XML file is 2310 KB, was created on Aug. 15, 2022, and is being submitted electronically via the USPTO patent electronic filing system.

### BACKGROUND

Antisense oligonucleotides (ASOs) are promising therapeutics being developed to treat diseases. ASOs were historically designed as single stranded nucleobase polymers to hybridize to a mRNA target, thereby reducing protein expression, e.g., by recruiting RNase H to cleave the mRNA. However, single stranded ASOs may induce unwanted side effects in nontargeted tissues or cells. Thus, there is a need to identify improved methods for targeted delivery of ASOs.
Greenberger et al. report an RNA antagonist of hypoxia-inducible factor-1α, EZN-2968, inhibits tumor cell growth. Molecular Cancer Therapeutics, 2008, 7(11):3598-3608.
Lewis et al. report regulation and biological function of the liver-specific miR-122. Biochem. Soc. Trans, 2010, 38, 1553-1557.
Zhang et al. report dynamic DNA nanotechnology using strand-displacement reactions. Nature Chemistry, 2011, 3(2):103.
Prakash et al. report targeted delivery of antisense oligonucleotides to hepatocytes using triantennary N-acetyl galactosamine. Nucleic acids research, 2014, 42(13):8796-8807.
Hanna et al. report the potential for microRNAs in clinical research. Frontiers in Genetics, 2019, 10, 478.
Esmerats et al., report disturbed flow increases UBE2C (Ubiquitin E2 Ligase C) via loss of miR-483-3p, inducing aortic valve calcification by the pVHL (von Hippel-Lindau protein) and HIF-1α (Hypoxia-Inducible Factor-la) pathway in endothelial cells. Arteriosclerosis, Thrombosis, and Vascular Biology, 2019, 39(3):467-481.

References cited herein are not an admission of prior art.

### SUMMARY

This disclosure relates to conditional double stranded antisense oligonucleotides and uses in managing diseases and conditions. In certain embodiments, the conditional double stranded antisense oligonucleotides are non-naturally occurring double stranded nucleobase polymer complexes comprising a first strand and a second strand, wherein the first strand is an antisense oligonucleotide connected to a segment that is partially identical to cell specific RNA, e.g., microRNA, and the second strand is a locking strand configured to release the first strand when in the presence of the cells specific RNA; thus, releasing the antisense oligonucleotide to alter the expression or degradation of a nucleic acid targeted by the antisense oligonucleotide in a manner limited to cells that express a specific RNA. In certain embodiments, the second strand contains a single stranded toehold segment that is designed to bind to the cell specific RNA facilitating the release of the first strand.

In certain embodiments, the conditional double stranded antisense oligonucleotides are non-naturally occurring double stranded nucleobase polymer complexes comprising a first strand comprising, a first domain with a first segment of a cell specific RNA and a second domain that is complementary to a segment of target RNA; and a second strand comprising, a first domain that is complementary to the second domain of the first strand, a second domain that is complementary to the first domain of the first strand, and a third domain that is complementary to a second segment of the cell specific RNA, wherein the third domain is not complementary to the first strand providing a single stranded toehold segment.

In certain embodiments, between the first domain of the first strand and the second domain of the first strand is one or more nucleobases that do not base pair with the second strand providing a bulge.

In certain embodiments, the cell specific RNA is microRNA. In certain embodiments, the cell specific RNA is hepatocyte-specific miRNA, miR-122. In certain embodiments, the target RNA is messenger RNA. In certain embodiments, the target RNA is HIF1alpha messenger RNA.

In certain embodiments, this disclosure contemplates a conditional double stranded antisense oligonucleotide disclosed herein comprising or conjugated to a label.

In certain embodiments, this disclosure contemplates a particle comprising a conditional double stranded antisense oligonucleotide disclosed herein.

In certain embodiments, this disclosure contemplates a pharmaceutical composition comprising conditional double stranded antisense oligonucleotide or particle comprising the same as disclosed herein and a pharmaceutically acceptable excipient.

In certain embodiments, this disclosure relates to methods of treating a disease or condition associated with target nucleic acid/RNA overexpression in a cell characterized by cell specific nucleic acid/RNA expression comprising administering to a subject in need thereof an effective amount of a double stranded nucleobase polymer complex or particle thereof as disclosed herein.

In certain embodiments, this disclosure relates to the production of a medicament for use in treating a disease or condition associated with target nucleic acid/RNA overexpression in a cell characterized by cell specific nucleic acid/RNA expression as disclosed herein.

### BRIEF DESCRIPTION OF THE SEVERAL VIEWS OF THE DRAWINGS

FIG. 1 illustrates a scheme showing the design and the triggering mechanism of conditional antisense oligonucleotides (ASOs). In this example, the conditional ASOs are formed by annealing a cell specific partial miRNA sequence and antisense oligonucleotide, i.e., pM-ASO strand and a locking strand. The pM-ASO strand is the parental ASO extended with partial miRNA sequence at its 5′ terminus. The locking stand comprises an anti-miRNA sequence and complementary sequence of the ASO. The mRNA targeting ASO sequence in the conditional ASO is sequestered, which abolishes its binding capability to the target mRNA in the absence of the trigger miRNA. The duplex can dissociate in the presence of trigger miRNA, exposing the mRNA targeting ASO sequence and causing the down regulation of the target mRNA. The triggered activation of conditional ASO is driven by toehold-mediated strand displacement. miRNA binds to the toehold region on the conditional ASO, which initiates branch migration and eventually leads to the dissociation of the pM-ASO strand and the locking strand. The activated pM-ASO strand can then bind to the target mRNA and cause its degradation.

FIG. 2A shows the design of conditional EZN2968 with end destabilization and bulge destabilization. For end destabilization, 3-7 nt on the 5′ termini of the locking strand were removed. For bulge destabilization, 3-7 nt in the middle of the locking strand were removed.

FIG. 2B shows a schematic description of in buffer assay to measure leakage activation triggered by a HIF1α mRNA mimicking sequence.

FIG. 2C shows a schematic description of miR-122 triggered activation of the conditional EZN2968. Cy5 and quencher were labeled on the conditional EZN2968 and the locking strand separately. The annealed duplex was incubated with a HIF1α mRNA mimicking sequence (mRNA mimic) or a miR-122 mimicking sequence (mRNA mimic).

FIG. 2D shows the fluorescence increases due to dequenching caused by displacement which was quantified with plate reader to calculate the percentage displacement. 10 nM unmodified conditional EZN2968 duplex was incubated with 100 nM HIF1α mRNA mimic at 37° C. for 2 h, and fluorescence intensity of Cy5 was measured to determine the percentage of displaced and activated pM-EZN strands. 10 nM unmodified conditional EZN2968 duplex was incubated with 100 nM miRNA mimic at 37° C. for 2 h, and fluorescence intensity of Cy5 was measured to determine the percentage of displaced and activated pM-EZN strands. Ratios of miR-122 triggered displacement and mRNA triggered displacement were determined.

FIG. 3A illustrates the structure and chemistry of conditional EZN2968. A conditional EZN2968 is composed of a pM-EZN strand and a locking strand. By tuning the length of the two strands, the duplexes with different toehold lengths and bulge sizes were created.

FIG. 3B shows data when HeLa cells were transfected with 10 nM of each duplex and incubated for 24 h. HIF1α mRNA levels were quantified by qPCR normalized to 18 s.

FIG. 3C shows data where LN229-V6R-Luc cells were transfected with 10 nM of each duplex and incubated for 4 h. 20 μM IOX4 was added in each well and incubated for another 20 h before luciferase assay was conducted to assess HIF1α activity.

FIG. 3D shows mean fluorescence intensity when HeLa cells were transfected with 10 nM Cy5-quencher labeled duplexes and incubated for 24 h, quantified by flow cytometry.

FIG. 4A shows data where U373 cells were co-transfected with 10 nM T10B0*, T7B0* or T7B3* and 500 nM miR-122 mimic. After 24 h incubation, HIF1α mRNA levels were quantified by qPCR normalized to 18 s.

FIG. 4B shows a western blot where U373 cells were cotransfected with 10 nM T7B3* and 500 nM miR-122 mimic and incubated for 24 h incubation. The cells were then lysed.

FIG. 4C shows data where HIF1α protein was quantified from the western blot.

FIG. 4D shows data where cells were co-transfected with 10 nM T7B3* and different concentrations of miR-122 mimic. After 24 h incubation, HIF1α mRNA levels were quantified by qPCR normalized to 18 s.

FIG. 4E shows data where U373 cells were co-transfected with 10 nM T7B3* with toehold or without toehold and 500 nM miR-122 mimic. After 24 h incubation, HIF1α mRNA levels were quantified by qPCR normalized to 18 s.

FIG. 4F shows data where U373 cells were co-transfected with 10 nM T7B3* and 100 nM miR-122 mimic or scr. 1-7nt miR-122. After 24 h incubation, HIF1α mRNA levels were quantified by qPCR normalized to 18 s.

FIG. 5A shows a scheme of fluorescence dequenching and fluorescence lifetime increase of Cy5 due to activation of T7B3* by miR-122 mimic.

FIG. 5B shows data on mean fluorescence intensity of U373 cells cotransfected with 10 nM T7B3* and 500 nM miR-122, scr. miR-122 and scr. 1-7nt miR-122 and incubated for 24 h.

FIG. 5C shows data of amplitude-averaged fluorescence lifetime of U373 cells transfected with 10 nM Cy5-Q-labeled T7B3* and 500 nM miR-122, scr. miR-122, or scr. 1-7nt miR-122, and incubated for 24 h. Cy5-labeled pM15-EZN or Cy5-labeled T7B3* transfected cells are positive controls, and Cy5-Q-labeled T7B3* transfected cells are negative controls.

FIG. 5D shows data of intensity-averaged fluorescence lifetime.

FIG. 6A shows data when Huh7 liver cells were transfected with 50 nM T7B3* with toehold or without toehold using Oligofectamine™. After 24 h incubation, HIF1α mRNA levels were quantified by qPCR normalized to 18 s.

FIG. 6B shows data when Huh7 cells were co-transfected with 50 nM T7B3* and 500 nM locking strand B3*. After 24 h incubation, HIF1α mRNA levels were quantified by qPCR normalized to 18 s.

FIG. 6C shows data when Huh7 cells were transfected with different concentrations of anti-miR-122 and 50 nM T7B3* sequentially with a 6 h interval. 24 h after the second transfection, HIF1α mRNA levels were quantified by qPCR normalized to 18 s.

FIG. 6D shows data when U373 cells were transfected with 10 nM miR-122- or miR-21-inducible T7B3* for 24 h. Corresponding pM15-EZN strands were transfected as positive controls. HIF1α mRNA levels were quantified by qPCR and normalized to 18 s.

FIG. 7 illustrates a conditional double stranded antisense oligonucleotide which is a non-naturally occurring double stranded nucleobase polymer complex (top). A first strand (1, partial miRNA/pM-ASO strand) comprises, a first domain (2, partial miRNA) with a nucleotide sequence identical to a first segment (10) of cell specific RNA, (8), e.g. miRNA, and a second domain (3, ASO) that is complementary to a segment of target RNA, e.g. mRNA; and a second strand (4, locking Strand) comprising, a first domain (5, comp-ASO) that is complementary to the second domain (3) of the first strand, a second domain (6, anti-partial miRNA) that is complementary the first domain (2) of the first strand, and a third domain (7, anti-miRNA) that is complementary to a second segment (9) of the cell specific RNA (8), wherein the third domain is not complementary to the first strand (1) providing single stranded segment (7, toehold). The first domain (2) of the first strand (1) is on the 5′ end of the first strand (1). The second domain (3) of the first strand (1) is on the 3′ end of the first strand (1). The first domain (5) of the second strand (4) is on the 5′ end of the second strand (4). The second domain (6) of the second strand (4) is on the 3′ end of the first domain (5) of the second strand (4). The third domain (7) of the second strand (4) is on the 3′-end of the second strand (4). The first segment (10) of cell specific RNA (8) is on the 3′ end. The second segment (9) of the cell specific RNA (8) is on the 5′ end.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The term “embodiment” refers to an example and does not infer that an invention is necessary limited to such example.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The term “comprising” in reference to a nucleic acid having a nucleotide sequence refers a nucleotide that may contain additional 5′-end or 3′-end nucleotides, i.e., the term is intended to include the nucleic acid sequence within a larger sequence. The term “consisting of” in reference to a nucleic acid having a nucleotide sequence refers a nucleic acid having the exact number of nucleotides in the sequence and not more or having not more than a rage of nucleotides expressly specified in the claim. In certain embodiments, the disclosure contemplates that the “5′-end” of a nucleic acid may consist of a nucleotide sequence,” which refers to the 5′-end of the nucleic acid having the exact number of nucleotides in the sequence and not more or having not more than a rage of nucleotides specified in the claim; however, the 3′-end may be connected to additional nucleotides, e.g., as part of a larger nucleic acid. In certain embodiments, the disclosure contemplates that the “3′-end” of a nucleic acid may consist of a nucleotide sequence,” which refers to the 3′-end of the nucleic acid having the exact number of nucleotides in the sequence and not more or having not more than a rage of nucleotides specified in the claim; however, the 5′-end may be connected to additional nucleotides, e.g., as part of a larger nucleic acid.

The term “inserting” into a cell refers to the process of introducing nucleobase polymers, nucleic acids, DNA, or RNA into the cytosol/cytoplasm of cells e.g., eukaryotic somatic cells, bypassing the cellular membrane. Phosphate backbones of DNA and RNA are negatively charged molecules and cell membranes are negatively charged. Thus, nucleic acids typically do not spontaneously pass-through cellular membranes. A variety of techniques are known in the art to move extracellular nucleic acid inside cells. Inserting nucleic acids into cells can be accomplished by mechanical means, e.g., microneedles/microinjection, electroporation, chemical, or biomolecular means, e.g., surrounding the nucleic acid in recombinant viral particles that release the interior components after cellular fusion and entry.

Cellular “transfection” refers to is the process of introducing nucleic acids into cells by chemical mechanism that interact with bilayer membranes. For example, calcium phosphate and diethylaminoethyl (DEAE)-dextran and cationic lipid-based reagents are able to coat nucleic acids, enabling the complexes of DNA:transfection reagents to cross cell membranes These complexes may be integrated into lipids formations/artificial liposomes. Cationic lipids are typically mixed with neutral lipids such as L-dioleoyl phosphatidylethanolamine to enhance fusion with lipid bilayers.

The term “specific binding agent” refers to a molecule, such as a protein, antibody, or nucleic acid, that binds a target molecule with a greater affinity than other random molecules, proteins, or nucleic acids. Examples of specific binding agents include antibodies that bind an epitope of an antigen or a receptor which binds a ligand. “Specifically binds” refers to the ability of a specific binding agent (such as an ligand, receptor, enzyme, nucleic acid, antibody or binding region/fragment thereof) to recognize and bind a target molecule such that its affinity (as determined by, e.g., affinity ELISA or other assays) is at least 10 times as great, but optionally 50 times as great, 100, 250 or 500 times as great, or even at least 1000 times as great or more as the affinity of the same for any other random molecule, nucleic acid, or polypeptide.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of covalent molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRn—, (C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups and alkoxyalkyl groups.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

A “fluorescent tag” or “fluorescent dye” refers to a compound that can re-emit electromagnetic radiation upon excitation with electromagnetic radiation (e.g., ultraviolet light) of a different wavelength. Typically, the emitted light has a longer wavelength (e.g., in visible spectrum) than the absorbed radiation. As the emitted light typically occurs almost simultaneously, i.e., in less than one second, when the absorbed radiation is in the invisible ultraviolet region of the spectrum, the emitted light may be in the visible region resulting in a distinctive identifiable color signal. Small molecule fluorescent tags typically contain several combined aromatic groups, or planar or cyclic molecules with multiple interconnected double bonds. Chen et al. report a variety of fluorescent tags that can be viewed across the visible spectrum. Nature Biotechnology, 2019, 37, 1287-1293. The term “fluorescent tag” is intended to include compounds of larger molecular weight such as natural fluorescent proteins, e.g., green fluorescent protein (GFP) and phycobiliproteins (PE, APC), and fluorescence particles such as quantum dots, e.g., preferably having 2-10 nm diameter.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

With regard to the nucleobases, it is contemplated that the term encompasses isobases, otherwise known as modified bases, e.g., are isoelectronic or have other substitutes configured to mimic naturally occurring hydrogen bonding base-pairs. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161). Within any of the sequences disclosed herein U may be substituted for T, or T may be substituted for U. U is one of the four nucleobases in the nucleic acid RNA. In DNA, the uracil (U) nucleobase is replaced by thymine (T). Uracil is a demethylated form of thymine. Thus, from a structural standpoint natural RNA is distinct from DNA due to the presence of a 2′ hydroxy on the ribose unit and demethylated thymine bases.

Nucleobase polymers may be chemically modified, e.g., within the sugar backbone or on the 5′ or 3′ ends. The nucleobase polymers can be modified, for example, with 2′-amino, 2′-O-allyl, 2′-fluoro, 2′-O-methyl, 2′-methyl, 2′-H of the ribose ring, or a locked nucleic acid. Locked nucleic acid (LNA) refers to oligonucleotides that contain one or more nucleobases in which an extra methylene bridge fixes the confirmation sugar moiety, e.g., in the C3′-endo (beta-D-LNA) or C2′-endo (alpha-L-LNA) conformation of ribose. Using locked nucleic acids within a nucleobase polymer typically increases the specific binding between a double stranded complex. In certain embodiments, the nucleobase polymer comprises locked monomers of 1-(hydroxymethyl)-2,5-dioxabicyclo[2.2.1]heptan-7-ol. In certain embodiments, nucleobase polymers are contemplated to comprise phosphorodiamidate morpholino oligomers (PMO).

In certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-methyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphonamidate, morpholin-2-ylmethanol, (2-(hydroxymethyl)morpholino)(piperazin-1-yl)phosphinate, or peptide nucleic acids or combinations thereof.

Conditional Antisense Oligonucleotide Triggered by miRNA

Antisense oligonucleotides (ASOs) are an emerging class of promising therapeutics which function by down regulating disease-related RNA. However, like all other oligonucleotide therapeutics, ASOs lack specificity to desired cell subtypes or tissues, which might induce unwanted effects in nontargeted tissues or cells. To provide finer control of ASO activity toward the goal of enhancing its specificity, a conditional ASO was designed that can be activated by a tissue/cell type specific transcript. The miR-122-inducible HIF1α ASO conditionally inhibits HIF1α in the presence of a hepatocyte-specific miRNA, miR-122, via toehold exchange reaction. The effect of conformation, thermostability, and chemical composition of the conditional ASO on its spontaneous activation and miR-122 triggered activation were evaluated. Knockdown of HIF1α expression by the conditional ASO upon triggering is demonstrated by both synthetic miR-122 mimic and endogenous miR-122 in vitro. The HIF1α knockdown mediated by the conditional ASO is due to separation and re-hybridization of the duplex, depending on toehold binding and miR-122 levels. The design principle of miRNA inducible ASO provides a method for developing other disease or tissue specific transcript inducible ASOs to enhance specificity and enhance controllability and safety of oligonucleotide therapeutics. Utilization of intracellular markers for controlling activity of ASOs expand the targeting competency to a broader range of clinical indications and provide better control over ASOs therapeutics.

To control oligonucleotide-mediated activity, a complementary sequence with high affinity against the oligonucleotide can be hybridized to it; thus, blocking its ability to bind the mRNA target, hence inhibiting its activity. ASO activity is inhibited via hybridization using a complementary sequence. De-hybridization of the duplex will rescue the ASO activity, which can be achieved via toehold-mediated displacement or exchange reaction triggered by an endogenous RNA transcript such as microRNAs (miRNAs). Expression of microRNAs change dynamically at different developmental and disease stages as well as in specific tissues and cell types. Here, miRNAs are contemplated as endogenous triggers to control ASO activity and enable mRNA knockdown in specific cell types or tissues.

Liver-specific microRNA (miR-122) triggers activation of a conditional HIF1α ASO reported herein. MicroRNA-122 is primarily expressed in hepatocytes and makes up to 70% and 52% of the total hepatic miRNA pool in adult mouse and human, respectively. HIF1α is a transcription factor that is related to diverse human diseases, such as cancer and cardiovascular diseases. However, because HIF1α is involved in a variety of cell activities and plays protective roles in wound healing and repairing acute injury as well as regulating neo-angiogenesis and tissue vascularization, systemically inhibiting of HIF1α may lead to side effects. Therefore, conditional regulation of HIF1α in targeted tissue or specific cell types could be beneficial.

A library of conditional ASOs were created and characterized. The library was tested to identify the design features that result in the most selective triggering of the ASO. The role of duplex architecture was investigated, including the length and spatial arrangement of single and double stranded domains, thermostability and chemical composition of the conditional ASO. Activation of the conditional ASOs were demonstrated by both a synthetic oligonucleotide mimicking miR-122 and endogenous miR-122 in vitro. The design principles of the conditional ASO discovered herein provide insights for the development of specific transcript inducible ASOs to enhance specificity of oligonucleotide therapeutics.

The main advantage of this design is the enhancement in specificity: allowing therapeutic oligonucleotides to exclusively down regulate gene expression transiently in a cell-type specific manner. Conditional ASO will be most desirable when target mRNAs are broadly expressed across many cell types but where selective inhibition would be desirable.

In addition to miRNAs, other cellular RNAs can be also used to trigger the conditional ASOs. Herein, miRNA was used as a trigger because of its expression is highly regulated in certain cell types and disease conditions, and because of its innate functions in binding to complementary nucleic acids. Conditional oligonucleotides can be delivered with other commonly used delivery methods, such as lipid conjugation, spherical nucleic acids, and other nanomaterial delivery vehicles, to facilitate cellular internalization.

Because dehybridization may occur for double-stranded conditional ASOs due to gradual dilution, bridging the 3′ terminus of the pM-ASO strand and the 5′ end of the locking strand could potentially reduce spontaneous activation, and provide reversibility of the conditional ASOs.

### Conditional Double Stranded Antisense Oligonucleotides

In certain embodiments, conditional double stranded antisense oligonucleotides are non-naturally occurring double stranded nucleobase polymer complexes comprising a first strand and a second strand, wherein the first strand is an antisense oligonucleotide connected to a segment that is partially identical to cell specific RNA, e.g., microRNA, and the second strand is a locking strand configured to release the first strand when in the presence of the cell specific RNA; thus, releasing the antisense oligonucleotide to alter the expression or degradation of a nucleic acid targeted by the antisense oligonucleotide in a manner limited to cells that express a specific RNA. In certain embodiments, the second strand contains a single stranded toehold segment that is designed to bind to the cell specific RNA facilitating the release of the first strand.

In certain embodiments, conditional double stranded antisense oligonucleotides are as illustrated in FIG. 7. In certain embodiments, the conditional double stranded antisense oligonucleotides are non-naturally occurring double stranded nucleobase polymer complexes comprising a first strand comprising, a first domain with a first segment of a cell specific RNA and a second domain that is complementary to a segment of target RNA; and a second strand comprising, a first domain that is complementary to the second domain of the first strand, a second domain that is complementary the first domain of the first strand second, and a third domain that is complementary to a second segment of the cell specific RNA, wherein the third domain is not complementary to the first strand providing a single stranded segment.

In certain embodiments, between the first domain of the first strand and the second domain of the first strand is one or more nucleobases that do not base pair with the second strand providing a bulge. In certain embodiments, the one or more nucleobases that do not base pair with the second strand are one, two, three, four, five or more nucleobases.

In certain embodiments, the after the second domain of the second strand there are one or more nucleobases that do not base pair with the second domain of the first strand providing a single stranded toehold segment on the 3′ end of the second strand.

In certain embodiments, the first domain of the first strand is on the 5′ end of the first strand. In certain embodiments, the second domain of the first strand is on the 3′ end of the first strand. In certain embodiments, the first domain of the second strand is on the 5′ end of the second strand. In certain embodiments, the second domain of the second strand is on the 3′ end of the first domain of the second strand. In certain embodiments, the third domain of the second strand is on the 3′ end of the second strand.

In certain embodiments, the cell specific RNA is microRNA. In certain embodiments, the cell specific RNA is hepatocyte-specific miRNA, miR-122. In certain embodiments, the microRNA is selected from: hsa-miR-576-3p (SEQ ID NO: 20), hsa-miR-140-5p (SEQ ID NO: 21), hsa-miR-522-5p (SEQ ID NO: 22), hsa-miR-1298-5p (SEQ ID NO: 23), hsa-miR-133a-3p (SEQ ID NO: 24), hsa-miR-4743-3p (SEQ ID NO: 25), hsa-miR-557 (SEQ ID NO: 26), hsa-miR-548ao-3p (SEQ ID NO: 27), hsa-miR-4649-5p (SEQ ID NO: 28), hsa-miR-5088-5p (SEQ ID NO: 29), hsa-miR-665 (SEQ ID NO: 30), hsa-miR-3622b-3p (SEQ ID NO: 31), hsa-miR-4493 (SEQ ID NO: 32), hsa-miR-1250-5p (SEQ ID NO: 33), hsa-miR-3622a-5p (SEQ ID NO: 34), hsa-miR-381-3p (SEQ ID NO: 35), hsa-miR-4709-3p (SEQ ID NO: 36), hsa-miR-6738-5p (SEQ ID NO: 37), hsa-miR-374b-3p (SEQ ID NO: 38), hsa-miR-7159-5p (SEQ ID NO: 39), hsa-miR-6874-3p (SEQ ID NO: 40), hsa-miR-448 (SEQ ID NO: 41), hsa-miR-548s (SEQ ID NO: 42), hsa-miR-515-5p (SEQ ID NO: 43), hsa-miR-6891-5p (SEQ ID NO: 44), hsa-miR-3940-5p (SEQ ID NO: 45), hsa-miR-431-3p (SEQ ID NO: 46), hsa-miR-103a-2-5p (SEQ ID NO: 47), hsa-miR-6832-5p (SEQ ID NO: 48), hsa-miR-6877-5p (SEQ ID NO: 49), hsa-miR-1298-3p (SEQ ID NO: 50), hsa-miR-3155a (SEQ ID NO: 51), hsa-miR-3682-5p (SEQ ID NO: 52), hsa-miR-5089-5p (SEQ ID NO: 53), hsa-miR-648 (SEQ ID NO: 54), hsa-miR-367-5p (SEQ ID NO: 55), hsa-miR-6740-5p (SEQ ID NO: 56), hsa-miR-450a-2-3p (SEQ ID NO: 57), hsa-miR-6793-3p (SEQ ID NO: 58), hsa-miR-541-5p (SEQ ID NO: 59), hsa-miR-3622a-3p (SEQ ID NO: 60), hsa-miR-6515-3p (SEQ ID NO: 61), hsa-miR-1276 (SEQ ID NO: 62), hsa-miR-6499-3p (SEQ ID NO: 63), hsa-miR-2276-3p (SEQ ID NO: 64), hsa-miR-3120-3p (SEQ ID NO: 65), hsa-miR-6780b-5p (SEQ ID NO: 66), hsa-miR-4769-5p (SEQ ID NO: 67), hsa-miR-1306-5p (SEQ ID NO: 68), hsa-miR-519b-3p (SEQ ID NO: 69), hsa-miR-489-3p (SEQ ID NO: 70), hsa-miR-23a-3p (SEQ ID NO: 71), hsa-miR-675-3p (SEQ ID NO: 72), hsa-miR-6796-5p (SEQ ID NO: 73), hsa-miR-1269a (SEQ ID NO: 74), hsa-miR-2110 (SEQ ID NO: 75), hsa-miR-483-3p (SEQ ID NO: 76), hsa-miR-4659a-3p (SEQ ID NO: 77), hsa-miR-1912-3p (SEQ ID NO: 78), hsa-miR-146a-5p (SEQ ID NO: 79), hsa-miR-1289 (SEQ ID NO: 80), hsa-miR-8485 (SEQ ID NO: 81), hsa-miR-563 (SEQ ID NO: 82), hsa-miR-425-3p (SEQ ID NO: 83), hsa-miR-4474-3p (SEQ ID NO: 84), hsa-miR-3672 (SEQ ID NO: 85), hsa-let-7a-3p (SEQ ID NO: 86), hsa-miR-5002-5p (SEQ ID NO: 87), hsa-miR-190b-3p (SEQ ID NO: 88), hsa-miR-4488 (SEQ ID NO: 89), hsa-miR-10527-5p (SEQ ID NO: 90), hsa-miR-3691-5p (SEQ ID NO: 91), hsa-miR-3688-5p (SEQ ID NO: 92), hsa-miR-532-3p (SEQ ID NO: 93), hsa-miR-4747-5p (SEQ ID NO: 94), hsa-miR-6813-3p (SEQ ID NO: 95), hsa-miR-3150b-3p (SEQ ID NO: 96), hsa-miR-6858-5p (SEQ ID NO: 97), hsa-miR-6768-3p (SEQ ID NO: 98), hsa-miR-4797-3p (SEQ ID NO: 99), hsa-miR-4684-5p (SEQ ID NO: 100), hsa-miR-4638-5p (SEQ ID NO: 101), hsa-miR-4300 (SEQ ID NO: 102), hsa-miR-6868-3p (SEQ ID NO: 103), hsa-miR-101-5p (SEQ ID NO: 104), hsa-miR-4650-3p (SEQ ID NO: 105), hsa-miR-548bc (SEQ ID NO: 106), hsa-miR-6771-3p (SEQ ID NO: 107), hsa-miR-6748-3p (SEQ ID NO: 108), hsa-miR-1248 (SEQ ID NO: 109), hsa-miR-4463 (SEQ ID NO: 110), hsa-miR-6794-5p (SEQ ID NO: 111), hsa-miR-4763-5p (SEQ ID NO: 112), hsa-miR-6132 (SEQ ID NO: 113), hsa-miR-1306-3p (SEQ ID NO: 114), hsa-miR-579-5p (SEQ ID NO: 115), hsa-miR-3917 (SEQ ID NO: 116), hsa-miR-4793-3p (SEQ ID NO: 117), hsa-miR-575 (SEQ ID NO: 118), hsa-miR-496 (SEQ ID NO: 119), hsa-miR-320c (SEQ ID NO: 120), hsa-miR-6818-3p UUGUCUCUUGUUCCUCACACAG (SEQ ID NO: 121), hsa-miR-661 (SEQ ID NO: 122), hsa-miR-3162-3p (SEQ ID NO: 123), hsa-miR-6797-5p (SEQ ID NO: 124), hsa-miR-5705 (SEQ ID NO: 125), hsa-miR-4722-5p (SEQ ID NO: 126), hsa-miR-4477a (SEQ ID NO: 127), hsa-miR-6767-3p (SEQ ID NO: 128), hsa-miR-424-3p (SEQ ID NO: 129), hsa-miR-499b-3p (SEQ ID NO: 130), hsa-miR-1277-5p (SEQ ID NO: 131), hsa-miR-1238-5p (SEQ ID NO: 132), hsa-miR-4799-5p (SEQ ID NO: 133), hsa-miR-3074-5p (SEQ ID NO: 134), hsa-miR-95-5p (SEQ ID NO: 135), hsa-miR-7157-5p (SEQ ID NO: 136), hsa-miR-4436a (SEQ ID NO: 137), hsa-miR-4634 (SEQ ID NO: 138), hsa-miR-10394-5p (SEQ ID NO: 139), hsa-miR-664b-5p (SEQ ID NO: 140), hsa-miR-4525 (SEQ ID NO: 141), hsa-miR-302c-5p (SEQ ID NO: 142), hsa-miR-486-3p (SEQ ID NO: 143), hsa-miR-449a (SEQ ID NO: 144), hsa-miR-5001-5p (SEQ ID NO: 145), hsa-miR-4765 (SEQ ID NO: 146), hsa-miR-3619-5p (SEQ ID NO: 147), hsa-miR-487b-3p (SEQ ID NO: 148), hsa-miR-766-3p (SEQ ID NO: 149), hsa-miR-7113-5p (SEQ ID NO: 150), hsa-miR-330-3p (SEQ ID NO: 151), hsa-miR-4257 (SEQ ID NO: 152), hsa-miR-4264 (SEQ ID NO: 153), hsa-miR-489-5p (SEQ ID NO: 154), hsa-miR-92b-5p (SEQ ID NO: 155), hsa-miR-6829-3p (SEQ ID NO: 156), hsa-miR-6766-3p (SEQ ID NO: 157), hsa-miR-5584-5p (SEQ ID NO: 158), hsa-miR-4710 (SEQ ID NO: 159), hsa-miR-3910 (SEQ ID NO: 160), hsa-miR-11400 (SEQ ID NO: 161), hsa-miR-10524-5p (SEQ ID NO: 162), hsa-miR-302e (SEQ ID NO: 163), hsa-miR-885-5p (SEQ ID NO: 164), hsa-miR-1282 (SEQ ID NO: 165), hsa-miR-4715-3p (SEQ ID NO: 166), hsa-miR-3124-5p (SEQ ID NO: 167), hsa-miR-103a-1-5p (SEQ ID NO: 168), hsa-miR-4750-5p (SEQ ID NO: 169), hsa-miR-191-3p (SEQ ID NO: 170), hsa-miR-6841-3p (SEQ ID NO: 171), hsa-miR-1301-5p (SEQ ID NO: 172), hsa-miR-3928-3p (SEQ ID NO: 173), hsa-miR-6799-5p (SEQ ID NO: 174), hsa-miR-20a-3p (SEQ ID NO: 175), hsa-miR-6787-3p (SEQ ID NO: 176), hsa-miR-1915-3p (SEQ ID NO: 177), hsa-miR-603 (SEQ ID NO: 178), hsa-miR-32-5p (SEQ ID NO: 179), hsa-miR-5706 (SEQ ID NO: 180), hsa-miR-6778-5p (SEQ ID NO: 181), hsa-miR-1301-3p (SEQ ID NO: 182), hsa-miR-3938 (SEQ ID NO: 183), hsa-miR-4777-5p (SEQ ID NO: 184), hsa-miR-371b-3p (SEQ ID NO: 185), hsa-miR-7112-3p (SEQ ID NO: 186), hsa-miR-370-3p (SEQ ID NO: 187), hsa-miR-574-3p (SEQ ID NO: 188), hsa-miR-4751 (SEQ ID NO: 189), hsa-miR-8071 (SEQ ID NO: 190), hsa-miR-23a-5p (SEQ ID NO: 191), hsa-miR-329-5p (SEQ ID NO: 192), hsa-miR-372-3p (SEQ ID NO: 193), hsa-miR-4479 (SEQ ID NO: 194), hsa-miR-105-3p (SEQ ID NO: 195), hsa-miR-4326 (SEQ ID NO: 196), hsa-let-7b-5p (SEQ ID NO: 197), hsa-miR-6751-5p (SEQ ID NO: 198), hsa-miR-3192-3p (SEQ ID NO: 199), hsa-miR-6856-3p (SEQ ID NO: 200), hsa-miR-6807-5p (SEQ ID NO: 201), hsa-miR-5680 (SEQ ID NO: 202), hsa-miR-6735-5p (SEQ ID NO: 203), hsa-miR-30a-5p (SEQ ID NO: 204), hsa-miR-1207-5p (SEQ ID NO: 205), hsa-miR-3189-3p (SEQ ID NO: 206), hsa-miR-4438 (SEQ ID NO: 207), hsa-miR-6844 (SEQ ID NO: 208), hsa-miR-6805-3p (SEQ ID NO: 209), hsa-miR-6880-5p (SEQ ID NO: 210), hsa-miR-302b-5p (SEQ ID NO: 211), hsa-miR-623 (SEQ ID NO: 212), hsa-miR-875-3p (SEQ ID NO: 213), hsa-miR-6869-3p (SEQ ID NO: 214), hsa-miR-657 (SEQ ID NO: 215), hsa-miR-4689 (SEQ ID NO: 216), hsa-miR-136-3p (SEQ ID NO: 217), hsa-miR-519a-3p (SEQ ID NO: 218), hsa-miR-9718 (SEQ ID NO: 219), hsa-miR-5003-3p (SEQ ID NO: 220), hsa-miR-576-5p (SEQ ID NO: 221), hsa-miR-371a-3p (SEQ ID NO: 222), hsa-miR-6762-3p (SEQ ID NO: 223), hsa-miR-586 (SEQ ID NO: 224), hsa-miR-621 (SEQ ID NO: 225), hsa-miR-6791-3p (SEQ ID NO: 226), hsa-miR-3150a-5p (SEQ ID NO: 227), hsa-miR-4436b-3p (SEQ ID NO: 228), hsa-miR-4664-5p (SEQ ID NO: 229), hsa-miR-3692-3p (SEQ ID NO: 230), hsa-miR-338-5p (SEQ ID NO: 231), hsa-miR-4693-3p (SEQ ID NO: 232), hsa-miR-543 (SEQ ID NO: 233), hsa-miR-151b (SEQ ID NO: 234), hsa-miR-211-3p (SEQ ID NO: 235), hsa-miR-7150 (SEQ ID NO: 236), hsa-miR-4445-3p (SEQ ID NO: 237), hsa-miR-96-5p (SEQ ID NO: 238), hsa-miR-5579-5p (SEQ ID NO: 239), hsa-miR-593-5p (SEQ ID NO: 240), hsa-miR-887-3p (SEQ ID NO: 241), hsa-miR-3689a-5p (SEQ ID NO: 242), hsa-miR-618 (SEQ ID NO: 243), hsa-miR-6504-3p (SEQ ID NO: 244), hsa-miR-1228-3p (SEQ ID NO: 245), hsa-miR-4282 (SEQ ID NO: 246), hsa-miR-106a-3p (SEQ ID NO: 247), hsa-miR-491-5p (SEQ ID NO: 248), hsa-miR-4685-3p (SEQ ID NO: 249), hsa-miR-548a1 (SEQ ID NO: 250), hsa-miR-6765-3p (SEQ ID NO: 251), hsa-miR-511-3p (SEQ ID NO: 252), hsa-miR-4286 (SEQ ID NO: 253), hsa-miR-627-3p (SEQ ID NO: 254), hsa-miR-644a (SEQ ID NO: 255), hsa-miR-124-5p (SEQ ID NO: 256), hsa-miR-6887-3p (SEQ ID NO: 257), hsa-miR-4449 (SEQ ID NO: 258), hsa-miR-641 (SEQ ID NO: 259), hsa-miR-1285-3p (SEQ ID NO: 260), hsa-miR-1193 (SEQ ID NO: 261), hsa-miR-3153 (SEQ ID NO: 262), hsa-miR-30a-3p (SEQ ID NO: 263), hsa-miR-4312 (SEQ ID NO: 264), hsa-miR-525-5p (SEQ ID NO: 265), hsa-miR-5006-5p (SEQ ID NO: 266), hsa-miR-203a-5p (SEQ ID NO: 267), hsa-miR-6733-3p (SEQ ID NO: 268), hsa-miR-4711-3p (SEQ ID NO: 269), hsa-miR-20b-5p (SEQ ID NO: 270), hsa-miR-5699-5p (SEQ ID NO: 271), hsa-miR-1260a (SEQ ID NO: 272), hsa-miR-5692c (SEQ ID NO: 273), hsa-miR-3161 (SEQ ID NO: 274), hsa-miR-6763-3p (SEQ ID NO: 275), hsa-miR-92a-1-5p (SEQ ID NO: 276), hsa-miR-548 h-3p (SEQ ID NO: 277), hsa-miR-4297 (SEQ ID NO: 278), hsa-miR-4457 (SEQ ID NO: 279), hsa-miR-3680-3p (SEQ ID NO: 280), hsa-miR-2392 (SEQ ID NO: 281), hsa-miR-548t-3p (SEQ ID NO: 282), hsa-miR-4784 (SEQ ID NO: 283), hsa-miR-1273c (SEQ ID NO: 284), hsa-miR-2682-3p (SEQ ID NO: 285), hsa-miR-7112-5p (SEQ ID NO: 286), hsa-miR-5690 (SEQ ID NO: 287), hsa-miR-5011-3p (SEQ ID NO: 288), hsa-miR-4431 (SEQ ID NO: 289), hsa-miR-4418 (SEQ ID NO: 290), hsa-miR-891a-5p (SEQ ID NO: 291), hsa-miR-378f (SEQ ID NO: 292), hsa-miR-183-3p (SEQ ID NO: 293), hsa-miR-3119 (SEQ ID NO: 294), hsa-miR-4640-3p (SEQ ID NO: 295), hsa-miR-3606-3p (SEQ ID NO: 296), hsa-miR-106b-5p (SEQ ID NO: 297), hsa-miR-4712-5p (SEQ ID NO: 298), hsa-miR-379-5p (SEQ ID NO: 299), hsa-miR-4678 (SEQ ID NO: 300), hsa-miR-3126-5p (SEQ ID NO: 301), hsa-miR-6069 (SEQ ID NO: 302), hsa-miR-1255b-5p (SEQ ID NO: 303), hsa-miR-6715a-3p (SEQ ID NO: 304), hsa-miR-4276 (SEQ ID NO: 305), hsa-miR-6812-3p (SEQ ID NO: 306), hsa-miR-4680-3p (SEQ ID NO: 307), hsa-let-7g-5p (SEQ ID NO: 308), hsa-miR-4757-3p (SEQ ID NO: 309), hsa-miR-4731-3p (SEQ ID NO: 310), hsa-miR-4645-5p (SEQ ID NO: 311), hsa-miR-4304 (SEQ ID NO: 312), hsa-miR-3658 (SEQ ID NO: 313), hsa-miR-4511 (SEQ ID NO: 314), hsa-miR-197-5p (SEQ ID NO: 315), hsa-miR-34b-3p (SEQ ID NO: 316), hsa-miR-6846-5p (SEQ ID NO: 317), hsa-miR-580-5p (SEQ ID NO: 318), hsa-miR-519e-5p (SEQ ID NO: 319), hsa-miR-106a-5p (SEQ ID NO: 320), hsa-miR-6715b-3p (SEQ ID NO: 321), hsa-miR-494-5p (SEQ ID NO: 322), hsa-miR-12124 (SEQ ID NO: 323), hsa-miR-4714-5p (SEQ ID NO: 324), hsa-miR-599 (SEQ ID NO: 325), hsa-miR-5697 (SEQ ID NO: 326), hsa-miR-216b-3p (SEQ ID NO: 327), hsa-miR-3692-5p (SEQ ID NO: 328), hsa-miR-4682 (SEQ ID NO: 329), hsa-miR-4519 (SEQ ID NO: 330), hsa-miR-6801-5p (SEQ ID NO: 331), hsa-miR-548v (SEQ ID NO: 332), hsa-miR-3661 (SEQ ID NO: 333), hsa-miR-4791 (SEQ ID NO: 334), hsa-miR-4638-3p (SEQ ID NO: 335), hsa-miR-214-5p (SEQ ID NO: 336), hsa-miR-513b-3p (SEQ ID NO: 337), hsa-miR-1296-5p (SEQ ID NO: 338), hsa-miR-6500-3p (SEQ ID NO: 339), hsa-miR-635 (SEQ ID NO: 340), hsa-miR-6892-5p (SEQ ID NO: 341), hsa-miR-328-3p (SEQ ID NO: 342), hsa-miR-372-5p (SEQ ID NO: 343), hsa-miR-1291 (SEQ ID NO: 344), hsa-miR-3912-3p (SEQ ID NO: 345), hsa-miR-526a-5p (SEQ ID NO: 346), hsa-miR-4673 (SEQ ID NO: 347), hsa-miR-885-3p (SEQ ID NO: 348), hsa-miR-10522-5p (SEQ ID NO: 349), hsa-miR-6075 (SEQ ID NO: 350), hsa-miR-495-5p (SEQ ID NO: 351), hsa-miR-4759 (SEQ ID NO: 352), hsa-miR-141-5p (SEQ ID NO: 353), hsa-miR-4484 (SEQ ID NO: 354), hsa-miR-643 (SEQ ID NO: 355), hsa-miR-4676-3p (SEQ ID NO: 356), hsa-miR-3169 (SEQ ID NO: 357), hsa-miR-4735-5p (SEQ ID NO: 358), hsa-miR-4756-3p (SEQ ID NO: 359), hsa-miR-1908-5p (SEQ ID NO: 360), hsa-miR-2277-3p (SEQ ID NO: 361), hsa-miR-3162-5p (SEQ ID NO: 362), hsa-miR-551b-3p (SEQ ID NO: 363), hsa-miR-5739 (SEQ ID NO: 364), hsa-miR-6893-5p (SEQ ID NO: 365), hsa-miR-548ae-3p (SEQ ID NO: 366), hsa-miR-6821-3p (SEQ ID NO: 367), hsa-miR-378i (SEQ ID NO: 368), hsa-miR-323b-5p (SEQ ID NO: 369), hsa-miR-4539 (SEQ ID NO: 370), hsa-miR-375-3p (SEQ ID NO: 371), hsa-miR-518a-5p (SEQ ID NO: 372), hsa-miR-608 (SEQ ID NO: 373), hsa-miR-770-5p (SEQ ID NO: 374), hsa-miR-6514-5p (SEQ ID NO: 375), hsa-miR-3615 (SEQ ID NO: 376), hsa-miR-3621 (SEQ ID NO: 377), hsa-miR-4508 (SEQ ID NO: 378), hsa-miR-6839-3p (SEQ ID NO: 379), hsa-miR-4698 (SEQ ID NO: 380), hsa-miR-4521 (SEQ ID NO: 381), hsa-miR-33a-5p (SEQ ID NO: 382), hsa-miR-1287-5p (SEQ ID NO: 383), hsa-miR-4650-5p (SEQ ID NO: 384), hsa-miR-154-5p (SEQ ID NO: 385), hsa-miR-1912-5p (SEQ ID NO: 386), hsa-miR-449b-3p (SEQ ID NO: 387), hsa-miR-6857-3p (SEQ ID NO: 388), hsa-miR-3617-3p (SEQ ID NO: 389), hsa-miR-4253 (SEQ ID NO: 390), hsa-miR-216b-5p (SEQ ID NO: 391), hsa-miR-6817-3p (SEQ ID NO: 392), hsa-miR-1226-3p (SEQ ID NO: 393), hsa-miR-127-3p (SEQ ID NO: 394), hsa-miR-2681-5p (SEQ ID NO: 395), hsa-miR-23b-3p (SEQ ID NO: 396), hsa-miR-5004-3p (SEQ ID NO: 397), hsa-miR-3613-5p (SEQ ID NO: 398), hsa-miR-3614-5p (SEQ ID NO: 399), hsa-miR-10394-3p (SEQ ID NO: 400), hsa-miR-6779-3p (SEQ ID NO: 401), hsa-miR-718 (SEQ ID NO: 402), hsa-miR-4718 (SEQ ID NO: 403), hsa-miR-98-3p (SEQ ID NO: 404), hsa-miR-325 (SEQ ID NO: 405), hsa-miR-6795-3p (SEQ ID NO: 406), hsa-miR-5195-5p (SEQ ID NO: 407), hsa-miR-6501-5p (SEQ ID NO: 408), hsa-miR-4520-5p (SEQ ID NO: 409), hsa-miR-4774-3p (SEQ ID NO: 410), hsa-miR-4323 (SEQ ID NO: 411), hsa-miR-4750-3p (SEQ ID NO: 412), hsa-miR-4665-5p (SEQ ID NO: 413), hsa-miR-302a-3p (SEQ ID NO: 414), hsa-miR-6790-3p (SEQ ID NO: 415), hsa-miR-148b-5p (SEQ ID NO: 416), hsa-miR-3129-5p (SEQ ID NO: 417), hsa-miR-4299 (SEQ ID NO: 418), hsa-miR-3972 (SEQ ID NO: 419), hsa-miR-1843 (SEQ ID NO: 420), hsa-miR-5197-5p (SEQ ID NO: 421), hsa-miR-1343-5p (SEQ ID NO: 422), hsa-miR-3960 (SEQ ID NO: 423), hsa-miR-4303 (SEQ ID NO: 424), hsa-miR-3132 (SEQ ID NO: 425), hsa-miR-31-5p (SEQ ID NO: 426), hsa-miR-518f-3p (SEQ ID NO: 427), hsa-miR-541-3p (SEQ ID NO: 428), hsa-miR-126-5p (SEQ ID NO: 429), hsa-miR-6825-3p (SEQ ID NO: 430), hsa-miR-6770-3p (SEQ ID NO: 431), hsa-miR-4420 (SEQ ID NO: 432), hsa-miR-488-5p (SEQ ID NO: 433), hsa-miR-1258 (SEQ ID NO: 434), hsa-miR-3138 (SEQ ID NO: 435), hsa-miR-4662b (SEQ ID NO: 436), hsa-miR-6881-3p (SEQ ID NO: 437), hsa-miR-3142 (SEQ ID NO: 438), hsa-miR-6775-3p (SEQ ID NO: 439), hsa-miR-6864-5p (SEQ ID NO: 440), hsa-miR-6721-5p (SEQ ID NO: 441), hsa-miR-6782-3p (SEQ ID NO: 442), hsa-miR-7106-5p (SEQ ID NO: 443), hsa-miR-139-3p (SEQ ID NO: 444), hsa-miR-554 (SEQ ID NO: 445), hsa-miR-491-3p (SEQ ID NO: 446), hsa-miR-4659a-5p (SEQ ID NO: 447), hsa-miR-4778-3p (SEQ ID NO: 448), hsa-miR-6882-3p (SEQ ID NO: 449), hsa-miR-4261 (SEQ ID NO: 450), hsa-miR-6754-5p (SEQ ID NO: 451), hsa-miR-376c-3p (SEQ ID NO: 452), hsa-miR-12114 (SEQ ID NO: 453), hsa-miR-6826-3p (SEQ ID NO: 454), hsa-miR-30b-5p (SEQ ID NO: 455), hsa-miR-6867-3p (SEQ ID NO: 456), hsa-miR-5687 (SEQ ID NO: 457), hsa-miR-9-3p (SEQ ID NO: 458), hsa-miR-2681-3p (SEQ ID NO: 459), hsa-miR-6858-3p (SEQ ID NO: 460), hsa-miR-4697-5p (SEQ ID NO: 461), hsa-miR-3684 (SEQ ID NO: 462), hsa-miR-634 (SEQ ID NO: 463), hsa-miR-208a-5p (SEQ ID NO: 464), hsa-miR-12133 (SEQ ID NO: 465), hsa-miR-567 (SEQ ID NO: 466), hsa-miR-3191-3p (SEQ ID NO: 467), hsa-miR-4524b-5p (SEQ ID NO: 468), hsa-miR-6726-5p (SEQ ID NO: 469), hsa-miR-2052 (SEQ ID NO: 470), hsa-miR-6797-3p (SEQ ID NO: 471), hsa-miR-1914-5p (SEQ ID NO: 472), hsa-miR-330-5p (SEQ ID NO: 473), hsa-miR-3197 (SEQ ID NO: 474), hsa-miR-139-5p (SEQ ID NO: 475), hsa-miR-10401-3p (SEQ ID NO: 476), hsa-miR-941 (SEQ ID NO: 477), hsa-miR-6761-5p (SEQ ID NO: 478), hsa-miR-4795-5p (SEQ ID NO: 479), hsa-miR-301a-5p (SEQ ID NO: 480), hsa-miR-181b-3p (SEQ ID NO: 481), hsa-miR-5047 (SEQ ID NO: 482), hsa-miR-548ap-3p (SEQ ID NO: 483), hsa-miR-571 (SEQ ID NO: 484), hsa-miR-492 (SEQ ID NO: 485), hsa-miR-6855-3p (SEQ ID NO: 486), hsa-miR-340-3p (SEQ ID NO: 487), hsa-miR-3160-3p (SEQ ID NO: 488), hsa-miR-126-3p (SEQ ID NO: 489), hsa-miR-4446-5p (SEQ ID NO: 490), hsa-miR-4742-5p (SEQ ID NO: 491), hsa-miR-451a (SEQ ID NO: 492), hsa-miR-548ag (SEQ ID NO: 493), hsa-miR-3194-3p (SEQ ID NO: 494), hsa-miR-8076 (SEQ ID NO: 495), hsa-miR-7151-5p (SEQ ID NO: 496), hsa-miR-4709-5p (SEQ ID NO: 497), hsa-miR-2277-5p (SEQ ID NO: 498), hsa-miR-6784-5p (SEQ ID NO: 499), hsa-miR-3125 (SEQ ID NO: 500), hsa-miR-4476 (SEQ ID NO: 501), hsa-miR-103a-3p (SEQ ID NO: 502), hsa-miR-4498 (SEQ ID NO: 503), hsa-miR-4472 (SEQ ID NO: 504), hsa-let-7i-3p (SEQ ID NO: 505), hsa-miR-199b-5p (SEQ ID NO: 506), hsa-miR-383-3p (SEQ ID NO: 507), hsa-miR-5702 (SEQ ID NO: 508), hsa-miR-4696 (SEQ ID NO: 509), hsa-miR-8062 (SEQ ID NO: 510), hsa-miR-518e-3p (SEQ ID NO: 511), hsa-miR-7114-5p (SEQ ID NO: 512), hsa-miR-6821-5p (SEQ ID NO: 513), hsa-miR-6879-3p (SEQ ID NO: 514), hsa-miR-5580-5p (SEQ ID NO: 515), hsa-miR-8079 (SEQ ID NO: 516), hsa-miR-527 (SEQ ID NO: 517), hsa-miR-450a-1-3p (SEQ ID NO: 518), hsa-miR-4499 (SEQ ID NO: 519), hsa-miR-4781-5p (SEQ ID NO: 520), hsa-miR-1321 (SEQ ID NO: 521), hsa-miR-585-3p (SEQ ID NO: 522), hsa-miR-4305 (SEQ ID NO: 523), hsa-miR-2276-5p (SEQ ID NO: 524), hsa-miR-329-3p (SEQ ID NO: 525), hsa-miR-1915-5p (SEQ ID NO: 526), hsa-miR-224-5p (SEQ ID NO: 527), hsa-miR-6840-3p (SEQ ID NO: 528), hsa-miR-6072 (SEQ ID NO: 529), hsa-miR-6079 (SEQ ID NO: 530), hsa-miR-548ax (SEQ ID NO: 531), hsa-miR-6511b-5p (SEQ ID NO: 532), hsa-miR-144-3p (SEQ ID NO: 533), hsa-miR-4700-5p (SEQ ID NO: 534), hsa-miR-3945 (SEQ ID NO: 535), hsa-miR-4442 (SEQ ID NO: 536), hsa-miR-4713-5p (SEQ ID NO: 537), hsa-miR-520 h (SEQ ID NO: 538), hsa-miR-520e-3p (SEQ ID NO: 539), hsa-miR-499a-5p (SEQ ID NO: 540), hsa-miR-3616-3p (SEQ ID NO: 541), hsa-miR-4311 (SEQ ID NO: 542), hsa-miR-374a-3p (SEQ ID NO: 543), hsa-miR-4514 (SEQ ID NO: 544), hsa-miR-141-3p (SEQ ID NO: 545), hsa-miR-6856-5p (SEQ ID NO: 546), hsa-miR-339-5p (SEQ ID NO: 547), hsa-miR-186-5p (SEQ ID NO: 548), hsa-miR-486-5p (SEQ ID NO: 549), hsa-miR-3927-5p (SEQ ID NO: 550), hsa-miR-7515 (SEQ ID NO: 551), hsa-miR-548ay-5p (SEQ ID NO: 552), hsa-miR-548j-5p (SEQ ID NO: 553), hsa-miR-3936 (SEQ ID NO: 554), hsa-miR-4320 (SEQ ID NO: 555), hsa-miR-1273 h-5p (SEQ ID NO: 556), hsa-miR-4782-3p (SEQ ID NO: 557), hsa-miR-6823-3p (SEQ ID NO: 558), hsa-miR-218-5p (SEQ ID NO: 559), hsa-miR-3117-5p (SEQ ID NO: 560), hsa-miR-663b (SEQ ID NO: 561), hsa-miR-1279 (SEQ ID NO: 562), hsa-miR-4656 (SEQ ID NO: 563), hsa-miR-135a-5p (SEQ ID NO: 564), hsa-miR-3186-5p (SEQ ID NO: 565), hsa-miR-3922-5p (SEQ ID NO: 566), hsa-miR-6785-5p (SEQ ID NO: 567), hsa-miR-2053 (SEQ ID NO: 568), hsa-miR-376c-5p (SEQ ID NO: 569), hsa-miR-93-5p (SEQ ID NO: 570), hsa-miR-1587 (SEQ ID NO: 571), hsa-miR-6816-5p (SEQ ID NO: 572), hsa-miR-4452 (SEQ ID NO: 573), hsa-miR-548k (SEQ ID NO: 574), hsa-miR-6736-5p (SEQ ID NO: 575), hsa-miR-1236-5p (SEQ ID NO: 576), hsa-miR-764 (SEQ ID NO: 577), hsa-miR-10395-5p (SEQ ID NO: 578), hsa-miR-208b-5p (SEQ ID NO: 579), hsa-miR-6866-3p (SEQ ID NO: 580), hsa-miR-518f-5p (SEQ ID NO: 581), hsa-miR-4330 (SEQ ID NO: 582), hsa-miR-6511b-3p (SEQ ID NO: 583), hsa-miR-6750-5p (SEQ ID NO: 584), hsa-miR-4501 (SEQ ID NO: 585), hsa-miR-8067 (SEQ ID NO: 586), hsa-miR-196a-1-3p (SEQ ID NO: 587), hsa-miR-4803 (SEQ ID NO: 588), hsa-miR-887-5p (SEQ ID NO: 589), hsa-miR-6509-3p (SEQ ID NO: 590), hsa-miR-7107-5p (SEQ ID NO: 591), hsa-miR-4772-3p (SEQ ID NO: 592), hsa-miR-614 (SEQ ID NO: 593), hsa-miR-3193 (SEQ ID NO: 594), hsa-miR-1250-3p (SEQ ID NO: 595), hsa-miR-4783-3p (SEQ ID NO: 596), hsa-miR-10396a-5p (SEQ ID NO: 597), hsa-miR-6761-3p (SEQ ID NO: 598), hsa-miR-379-3p (SEQ ID NO: 599), hsa-miR-6734-3p (SEQ ID NO: 600), hsa-miR-892a (SEQ ID NO: 601), hsa-miR-1304-3p (SEQ ID NO: 602), hsa-miR-4530 (SEQ ID NO: 603), hsa-miR-4707-5p (SEQ ID NO: 604), hsa-miR-6068 (SEQ ID NO: 605), hsa-miR-24-1-5p (SEQ ID NO: 606), hsa-miR-3689e (SEQ ID NO: 607), hsa-miR-5584-3p (SEQ ID NO: 608), hsa-miR-5010-5p (SEQ ID NO: 609), hsa-miR-6125 (SEQ ID NO: 610), hsa-miR-4424 (SEQ ID NO: 611), hsa-miR-6835-3p (SEQ ID NO: 612), hsa-miR-7703 (SEQ ID NO: 613), hsa-miR-652-5p (SEQ ID NO: 614), hsa-miR-3691-3p (SEQ ID NO: 615), hsa-miR-4272 (SEQ ID NO: 616), hsa-miR-3183 (SEQ ID NO: 617), hsa-miR-6863 (SEQ ID NO: 618), hsa-miR-3942-3p (SEQ ID NO: 619), hsa-miR-1244 (SEQ ID NO: 620), hsa-miR-6812-5p (SEQ ID NO: 621), hsa-miR-373-5p (SEQ ID NO: 622), hsa-miR-556-3p (SEQ ID NO: 623), hsa-miR-564 (SEQ ID NO: 624), hsa-miR-548o-5p (SEQ ID NO: 625), hsa-miR-367-3p (SEQ ID NO: 626), hsa-miR-601 (SEQ ID NO: 627), hsa-miR-4327 (SEQ ID NO: 628), hsa-miR-4665-3p (SEQ ID NO: 629), hsa-miR-4437 (SEQ ID NO: 630), hsa-miR-548aq-3p (SEQ ID NO: 631), hsa-miR-5191 (SEQ ID NO: 632), hsa-miR-4738-3p (SEQ ID NO: 633), hsa-miR-876-3p (SEQ ID NO: 634), hsa-miR-3922-3p (SEQ ID NO: 635), hsa-miR-423-5p (SEQ ID NO: 636), hsa-miR-6792-3p (SEQ ID NO: 637), hsa-miR-9901 (SEQ ID NO: 638), hsa-miR-1272 (SEQ ID NO: 639), hsa-miR-200a-3p (SEQ ID NO: 640), hsa-miR-4434 (SEQ ID NO: 641), hsa-miR-6870-5p (SEQ ID NO: 642), hsa-miR-509-5p (SEQ ID NO: 643), hsa-miR-4313 (SEQ ID NO: 644), hsa-miR-944 (SEQ ID NO: 645), hsa-miR-3184-3p (SEQ ID NO: 646), hsa-miR-548e-3p (SEQ ID NO: 647), hsa-miR-29b-1-5p (SEQ ID NO: 648), hsa-miR-548ac (SEQ ID NO: 649), hsa-miR-1265 (SEQ ID NO: 650), hsa-miR-1343-3p (SEQ ID NO: 651), hsa-miR-515-3p (SEQ ID NO: 652), hsa-miR-5694 (SEQ ID NO: 653), hsa-miR-3166 (SEQ ID NO: 654), hsa-miR-137-5p (SEQ ID NO: 655), hsa-miR-10395-3p (SEQ ID NO: 656), hsa-miR-203b-5p (SEQ ID NO: 657), hsa-miR-410-3p (SEQ ID NO: 658), hsa-miR-4317 (SEQ ID NO: 659), hsa-miR-365b-3p (SEQ ID NO: 660), hsa-miR-3164 (SEQ ID NO: 661), hsa-miR-216a-5p (SEQ ID NO: 662), hsa-miR-514a-5p (SEQ ID NO: 663), hsa-miR-3190-5p (SEQ ID NO: 664), hsa-miR-6795-5p (SEQ ID NO: 665), hsa-miR-12126 (SEQ ID NO: 666), hsa-miR-4467 (SEQ ID NO: 667), hsa-miR-155-5p (SEQ ID NO: 668), hsa-miR-6734-5p (SEQ ID NO: 669), hsa-miR-1183 (SEQ ID NO: 670), hsa-miR-548x-3p (SEQ ID NO: 671), hsa-miR-3144-5p (SEQ ID NO: 672), hsa-miR-4694-3p (SEQ ID NO: 673), hsa-let-7d-3p (SEQ ID NO: 674), hsa-miR-11399 (SEQ ID NO: 675), hsa-miR-520g-5p (SEQ ID NO: 676), hsa-miR-7854-3p (SEQ ID NO: 677), hsa-miR-3652 (SEQ ID NO: 678), hsa-miR-1275 (SEQ ID NO: 679), hsa-miR-500a-3p (SEQ ID NO: 680), hsa-miR-498-5p (SEQ ID NO: 681), hsa-miR-552-3p (SEQ ID NO: 682), hsa-miR-22-3p (SEQ ID NO: 683), hsa-miR-219a-2-3p (SEQ ID NO: 684), hsa-miR-411-5p (SEQ ID NO: 685), hsa-miR-570-5p (SEQ ID NO: 686), hsa-miR-1288-5p (SEQ ID NO: 687), hsa-miR-6884-3p (SEQ ID NO: 688), hsa-miR-891b (SEQ ID NO: 689), hsa-miR-3606-5p (SEQ ID NO: 690), hsa-miR-1247-3p (SEQ ID NO: 691), hsa-miR-3147 (SEQ ID NO: 692), hsa-miR-5700 (SEQ ID NO: 693), hsa-miR-1252-3p (SEQ ID NO: 694), hsa-miR-4653-3p (SEQ ID NO: 695), hsa-miR-5090 (SEQ ID NO: 696), hsa-miR-365a-3p (SEQ ID NO: 697), hsa-miR-7154-3p (SEQ ID NO: 698), hsa-miR-3179 (SEQ ID NO: 699), hsa-miR-6792-5p (SEQ ID NO: 700), hsa-miR-3152-5p (SEQ ID NO: 701), hsa-miR-744-3p (SEQ ID NO: 702), hsa-miR-138-2-3p (SEQ ID NO: 703), hsa-miR-8084 (SEQ ID NO: 704), hsa-miR-5707 (SEQ ID NO: 705), hsa-miR-8081 (SEQ ID NO: 706), hsa-miR-502-3p (SEQ ID NO: 707), hsa-miR-3937 (SEQ ID NO: 708), hsa-miR-3064-5p (SEQ ID NO: 709), hsa-miR-509-3-5p (SEQ ID NO: 710), hsa-miR-889-5p (SEQ ID NO: 711), hsa-miR-6745 (SEQ ID NO: 712), hsa-miR-3918 (SEQ ID NO: 713), hsa-miR-4755-5p (SEQ ID NO: 714), hsa-miR-3619-3p (SEQ ID NO: 715), hsa-miR-500b-5p (SEQ ID NO: 716), hsa-miR-192-3p (SEQ ID NO: 717), hsa-miR-4296 (SEQ ID NO: 718), hsa-miR-873-5p (SEQ ID NO: 719), hsa-miR-517a-3p (SEQ ID NO: 720), hsa-miR-1266-3p (SEQ ID NO: 721), hsa-miR-1233-5p (SEQ ID NO: 722), hsa-miR-2114-5p (SEQ ID NO: 723), hsa-miR-222-5p (SEQ ID NO: 724), hsa-miR-542-3p (SEQ ID NO: 725), hsa-miR-4768-5p (SEQ ID NO: 726), hsa-miR-589-5p (SEQ ID NO: 727), hsa-miR-12116 (SEQ ID NO: 728), hsa-miR-16-1-3p (SEQ ID NO: 729), hsa-miR-517b-3p (SEQ ID NO: 730), hsa-miR-181c-3p (SEQ ID NO: 731), hsa-miR-624-3p (SEQ ID NO: 732), hsa-miR-548o-3p (SEQ ID NO: 733), hsa-miR-4777-3p (SEQ ID NO: 734), hsa-miR-6766-5p (SEQ ID NO: 735), hsa-miR-181a-2-3p (SEQ ID NO: 736), hsa-miR-6529-5p (SEQ ID NO: 737), hsa-miR-6814-5p (SEQ ID NO: 738), hsa-miR-4732-3p (SEQ ID NO: 739), hsa-miR-335-3p (SEQ ID NO: 740), hsa-miR-5581-5p (SEQ ID NO: 741), hsa-miR-875-5p (SEQ ID NO: 742), hsa-miR-27a-5p (SEQ ID NO: 743), hsa-miR-942-3p (SEQ ID NO: 744), hsa-miR-4269 (SEQ ID NO: 745), hsa-miR-376a-2-5p (SEQ ID NO: 746), hsa-miR-1245a (SEQ ID NO: 747), hsa-miR-650 (SEQ ID NO: 748), hsa-miR-6884-5p (SEQ ID NO: 749), hsa-miR-1184 (SEQ ID NO: 750), hsa-miR-4256 (SEQ ID NO: 751), hsa-miR-517-5p (SEQ ID NO: 752), hsa-miR-4480 (SEQ ID NO: 753), hsa-miR-4635 (SEQ ID NO: 754), hsa-miR-4531 (SEQ ID NO: 755), hsa-miR-6892-3p (SEQ ID NO: 756), hsa-miR-4797-5p (SEQ ID NO: 757), hsa-miR-18b-5p (SEQ ID NO: 758), hsa-miR-18a-5p (SEQ ID NO: 759), hsa-miR-4778-5p (SEQ ID NO: 760), hsa-miR-3943 (SEQ ID NO: 761), hsa-miR-6773-3p (SEQ ID NO: 762), hsa-miR-6507-3p (SEQ ID NO: 763), hsa-miR-7-1-3p (SEQ ID NO: 764), hsa-miR-378j (SEQ ID NO: 765), hsa-miR-6839-5p (SEQ ID NO: 766), hsa-miR-6788-3p (SEQ ID NO: 767), hsa-miR-337-5p (SEQ ID NO: 768), hsa-miR-4800-3p (SEQ ID NO: 769), hsa-miR-374a-5p (SEQ ID NO: 770), hsa-miR-6513-3p (SEQ ID NO: 771), hsa-miR-4524a-5p (SEQ ID NO: 772), hsa-miR-28-3p (SEQ ID NO: 773), hsa-miR-4640-5p (SEQ ID NO: 774), hsa-miR-3611 (SEQ ID NO: 775), hsa-miR-8070 (SEQ ID NO: 776), hsa-miR-10a-5p (SEQ ID NO: 777), hsa-miR-29b-2-5p (SEQ ID NO: 778), hsa-miR-4727-3p (SEQ ID NO: 779), hsa-miR-561-3p (SEQ ID NO: 780), hsa-miR-514b-5p (SEQ ID NO: 781), hsa-miR-638 (SEQ ID NO: 782), hsa-miR-6516-5p (SEQ ID NO: 783), hsa-miR-3146 (SEQ ID NO: 784), hsa-miR-4707-3p (SEQ ID NO: 785), hsa-miR-6860 (SEQ ID NO: 786), hsa-miR-10396b-3p (SEQ ID NO: 787), hsa-miR-4267 (SEQ ID NO: 788), hsa-miR-4325 (SEQ ID NO: 789), hsa-miR-510-5p (SEQ ID NO: 790), hsa-miR-5587-3p (SEQ ID NO: 791), hsa-miR-490-3p (SEQ ID NO: 792), hsa-miR-4726-5p (SEQ ID NO: 793), hsa-miR-647 (SEQ ID NO: 794), hsa-miR-4726-3p (SEQ ID NO: 795), hsa-miR-559 (SEQ ID NO: 796), hsa-miR-101-3p (SEQ ID NO: 797), hsa-miR-4644 (SEQ ID NO: 798), hsa-miR-6716-5p (SEQ ID NO: 799), hsa-miR-301b-5p (SEQ ID NO: 800), hsa-miR-127-5p (SEQ ID NO: 801), hsa-miR-3134 (SEQ ID NO: 802), hsa-miR-4663 (SEQ ID NO: 803), hsa-miR-193b-3p (SEQ ID NO: 804), hsa-miR-589-3p (SEQ ID NO: 805), hsa-miR-520e-5p (SEQ ID NO: 806), hsa-miR-6861-3p (SEQ ID NO: 807), hsa-miR-6722-5p (SEQ ID NO: 808), hsa-miR-7853-5p (SEQ ID NO: 809), hsa-miR-5194 (SEQ ID NO: 810), hsa-miR-520a-3p (SEQ ID NO: 811), hsa-miR-3192-5p (SEQ ID NO: 812), hsa-miR-597-3p (SEQ ID NO: 813), hsa-miR-6836-3p (SEQ ID NO: 814), hsa-miR-548x-5p (SEQ ID NO: 815), hsa-miR-6888-3p (SEQ ID NO: 816), hsa-miR-6793-5p (SEQ ID NO: 817), hsa-miR-8075 (SEQ ID NO: 818), hsa-miR-1471 (SEQ ID NO: 819), hsa-miR-5590-3p (SEQ ID NO: 820), hsa-miR-24-3p (SEQ ID NO: 821), hsa-miR-6825-5p (SEQ ID NO: 822), hsa-miR-3934-5p (SEQ ID NO: 823), hsa-miR-6508-5p (SEQ ID NO: 824), hsa-miR-1238-3p (SEQ ID NO: 825), hsa-miR-598-3p (SEQ ID NO: 826), hsa-miR-548an (SEQ ID NO: 827), hsa-miR-6773-5p (SEQ ID NO: 828), hsa-miR-134-5p (SEQ ID NO: 829), hsa-miR-7157-3p (SEQ ID NO: 830), hsa-miR-4512 (SEQ ID NO: 831), hsa-miR-6733-5p (SEQ ID NO: 832), hsa-miR-6749-5p (SEQ ID NO: 833), hsa-miR-182-3p (SEQ ID NO: 834), hsa-miR-625-3p (SEQ ID NO: 835), hsa-miR-616-5p (SEQ ID NO: 836), hsa-miR-138-5p (SEQ ID NO: 837), hsa-miR-4448 (SEQ ID NO: 838), hsa-miR-4430 (SEQ ID NO: 839), hsa-miR-3065-3p (SEQ ID NO: 840), hsa-miR-3117-3p (SEQ ID NO: 841), hsa-miR-6852-3p (SEQ ID NO: 842), hsa-miR-6505-5p (SEQ ID NO: 843), hsa-miR-365a-5p (SEQ ID NO: 844), hsa-miR-6816-3p (SEQ ID NO: 845), hsa-miR-539-3p (SEQ ID NO: 846), hsa-miR-380-3p (SEQ ID NO: 847), hsa-miR-4487 (SEQ ID NO: 848), hsa-miR-548n (SEQ ID NO: 849), hsa-miR-9851-3p (SEQ ID NO: 850), hsa-miR-122b-3p (SEQ ID NO: 851), hsa-miR-892b (SEQ ID NO: 852), hsa-miR-6805-5p (SEQ ID NO: 853), hsa-miR-3942-5p (SEQ ID NO: 854), hsa-miR-1245b-5p (SEQ ID NO: 855), hsa-let-7e-3p (SEQ ID NO: 856), hsa-miR-659-5p (SEQ ID NO: 857), hsa-miR-3191-5p (SEQ ID NO: 858), hsa-miR-6886-3p (SEQ ID NO: 859), hsa-miR-3180-3p (SEQ ID NO: 860), hsa-miR-2467-5p (SEQ ID NO: 861), hsa-miR-5688 (SEQ ID NO: 862), hsa-miR-6872-3p (SEQ ID NO: 863), hsa-miR-1288-3p (SEQ ID NO: 864), hsa-miR-3679-5p (SEQ ID NO: 865), hsa-miR-4494 (SEQ ID NO: 866), hsa-miR-6867-5p (SEQ ID NO: 867), hsa-miR-548c-5p (SEQ ID NO: 868), hsa-miR-128-1-5p (SEQ ID NO: 869), hsa-miR-7704 (SEQ ID NO: 870), hsa-miR-664b-3p (SEQ ID NO: 871), hsa-miR-15b-3p (SEQ ID NO: 872), hsa-miR-4697-3p (SEQ ID NO: 873), hsa-miR-4458 (SEQ ID NO: 874), hsa-miR-196b-5p (SEQ ID NO: 875), hsa-miR-4645-3p (SEQ ID NO: 876), hsa-miR-181b-5p (SEQ ID NO: 877), hsa-miR-219b-5p (SEQ ID NO: 878), hsa-miR-3934-3p (SEQ ID NO: 879), hsa-miR-4746-3p (SEQ ID NO: 880), hsa-miR-1266-5p (SEQ ID NO: 881), hsa-miR-6791-5p (SEQ ID NO: 882), hsa-miR-409-5p (SEQ ID NO: 883), hsa-miR-508-5p (SEQ ID NO: 884), hsa-miR-3909 (SEQ ID NO: 885), hsa-miR-4504 (SEQ ID NO: 886), hsa-miR-6850-5p (SEQ ID NO: 887), hsa-miR-3683 (SEQ ID NO: 888), hsa-miR-587 (SEQ ID NO: 889), hsa-miR-4469 (SEQ ID NO: 890), hsa-miR-6804-3p (SEQ ID NO: 891), hsa-miR-510-3p (SEQ ID NO: 892), hsa-miR-624-5p (SEQ ID NO: 893), hsa-miR-548ap-5p (SEQ ID NO: 894), hsa-miR-193b-5p (SEQ ID NO: 895), hsa-miR-3151-3p (SEQ ID NO: 896), hsa-miR-1304-5p (SEQ ID NO: 897), hsa-miR-6074 (SEQ ID NO: 898), hsa-miR-7155-3p (SEQ ID NO: 899), hsa-miR-550a-5p (SEQ ID NO: 900), hsa-miR-8087 (SEQ ID NO: 901), hsa-miR-6510-5p (SEQ ID NO: 902), hsa-miR-3605-5p (SEQ ID NO: 903), hsa-miR-1268b (SEQ ID NO: 904), hsa-miR-6129 (SEQ ID NO: 905), hsa-miR-6857-5p (SEQ ID NO: 906), hsa-miR-215-3p (SEQ ID NO: 907), hsa-miR-10398-5p (SEQ ID NO: 908), hsa-miR-30b-3p (SEQ ID NO: 909), hsa-miR-4687-3p (SEQ ID NO: 910), hsa-miR-519a-2-5p (SEQ ID NO: 911), hsa-miR-4794 (SEQ ID NO: 912), hsa-miR-6727-3p (SEQ ID NO: 913), hsa-miR-516a-5p (SEQ ID NO: 914), hsa-miR-6806-5p (SEQ ID NO: 915), hsa-miR-29b-3p (SEQ ID NO: 916), hsa-miR-4713-3p (SEQ ID NO: 917), hsa-miR-12123 (SEQ ID NO: 918), hsa-miR-4433b-5p (SEQ ID NO: 919), hsa-miR-6755-3p (SEQ ID NO: 920), hsa-miR-4659b-3p (SEQ ID NO: 921), hsa-miR-6513-5p (SEQ ID NO: 922), hsa-miR-8052 (SEQ ID NO: 923), hsa-miR-6872-5p (SEQ ID NO: 924), hsa-miR-431-5p (SEQ ID NO: 925), hsa-miR-5011-5p (SEQ ID NO: 926), hsa-miR-7974 (SEQ ID NO: 927), hsa-miR-548ar-5p (SEQ ID NO: 928), hsa-miR-200b-3p (SEQ ID NO: 929), hsa-miR-6768-5p (SEQ ID NO: 930), hsa-miR-6516-3p (SEQ ID NO: 931), hsa-miR-1249-5p (SEQ ID NO: 932), hsa-miR-3201 (SEQ ID NO: 933), hsa-miR-6499-5p (SEQ ID NO: 934), hsa-miR-1305 (SEQ ID NO: 935), hsa-miR-937-3p (SEQ ID NO: 936), hsa-miR-432-3p (SEQ ID NO: 937), hsa-miR-6870-3p (SEQ ID NO: 938), hsa-miR-6510-3p (SEQ ID NO: 939), hsa-miR-3660 (SEQ ID NO: 940), hsa-miR-550b-3p (SEQ ID NO: 941), hsa-miR-606 (SEQ ID NO: 942), hsa-miR-421 (SEQ ID NO: 943), hsa-miR-519e-3p (SEQ ID NO: 944), hsa-miR-3180 (SEQ ID NO: 945), hsa-miR-151a-5p (SEQ ID NO: 946), hsa-miR-378g (SEQ ID NO: 947), hsa-miR-29a-5p (SEQ ID NO: 948), hsa-miR-6891-3p (SEQ ID NO: 949), hsa-miR-521 (SEQ ID NO: 950), hsa-miR-6894-3p (SEQ ID NO: 951), hsa-miR-518c-5p (SEQ ID NO: 952), hsa-miR-646 (SEQ ID NO: 953), hsa-miR-4788 (SEQ ID NO: 954), hsa-miR-590-5p (SEQ ID NO: 955), hsa-miR-1281 (SEQ ID NO: 956), hsa-miR-4483 (SEQ ID NO: 957), hsa-miR-5192 (SEQ ID NO: 958), hsa-miR-3657 (SEQ ID NO: 959), hsa-miR-148a-3p (SEQ ID NO: 960), hsa-miR-3151-5p (SEQ ID NO: 961), hsa-miR-4691-5p (SEQ ID NO: 962), hsa-miR-8054 (SEQ ID NO: 963), hsa-miR-412-5p (SEQ ID NO: 964), hsa-miR-4482-3p (SEQ ID NO: 965), hsa-miR-6851-3p (SEQ ID NO: 966), hsa-miR-6502-5p (SEQ ID NO: 967), hsa-miR-570-3p (SEQ ID NO: 968), hsa-miR-2113 (SEQ ID NO: 969), hsa-miR-6736-3p (SEQ ID NO: 970), hsa-miR-3135a (SEQ ID NO: 971), hsa-miR-4757-5p (SEQ ID NO: 972), hsa-miR-324-3p (SEQ ID NO: 973), hsa-miR-622 (SEQ ID NO: 974), hsa-miR-5571-3p (SEQ ID NO: 975), hsa-miR-581 (SEQ ID NO: 976), hsa-miR-7156-3p (SEQ ID NO: 977), hsa-miR-520g-3p (SEQ ID NO: 978), hsa-miR-1207-3p (SEQ ID NO: 979), hsa-miR-506-3p (SEQ ID NO: 980), hsa-miR-6811-5p (SEQ ID NO: 981), hsa-miR-6775-5p (SEQ ID NO: 982), hsa-miR-6748-5p (SEQ ID NO: 983), hsa-miR-1273 h-3p (SEQ ID NO: 984), hsa-miR-31-3p (SEQ ID NO: 985), hsa-miR-676-5p (SEQ ID NO: 986), hsa-miR-2115-3p (SEQ ID NO: 987), hsa-miR-7152-5p (SEQ ID NO: 988), hsa-miR-4460 (SEQ ID NO: 989), hsa-miR-5681a (SEQ ID NO: 990), hsa-miR-3974 (SEQ ID NO: 991), hsa-miR-6083 (SEQ ID NO: 992), hsa-miR-7156-5p (SEQ ID NO: 993), hsa-miR-6780a-5p (SEQ ID NO: 994), hsa-miR-6802-3p (SEQ ID NO: 995), hsa-miR-484 (SEQ ID NO: 996), hsa-miR-382-3p (SEQ ID NO: 997), hsa-miR-6770-5p (SEQ ID NO: 998), hsa-miR-4701-5p (SEQ ID NO: 999), hsa-miR-4752 (SEQ ID NO: 1000), hsa-miR-6848-5p (SEQ ID NO: 1001), hsa-miR-6724-5p (SEQ ID NO: 1002), hsa-miR-124-3p (SEQ ID NO: 1003), hsa-miR-3923 (SEQ ID NO: 1004), hsa-miR-4422 (SEQ ID NO: 1005), hsa-miR-96-3p (SEQ ID NO: 1006), hsa-miR-4441 (SEQ ID NO: 1007), hsa-miR-4535 (SEQ ID NO: 1008), hsa-miR-6831-3p (SEQ ID NO: 1009), hsa-miR-4676-5p (SEQ ID NO: 1010), hsa-miR-1324 (SEQ ID NO: 1011), hsa-miR-320d (SEQ ID NO: 1012), hsa-miR-526b-5p (SEQ ID NO: 1013), hsa-miR-3620-5p (SEQ ID NO: 1014), hsa-miR-3926 (SEQ ID NO: 1015), hsa-miR-95-3p (SEQ ID NO: 1016), hsa-miR-4688 (SEQ ID NO: 1017), hsa-miR-222-3p (SEQ ID NO: 1018), hsa-miR-617 (SEQ ID NO: 1019), hsa-miR-4285 (SEQ ID NO: 1020), hsa-miR-6865-3p (SEQ ID NO: 1021), hsa-miR-1178-3p (SEQ ID NO: 1022), hsa-miR-4796-5p (SEQ ID NO: 1023), hsa-miR-1297 (SEQ ID NO: 1024), hsa-miR-7852-3p (SEQ ID NO: 1025), hsa-miR-1208 (SEQ ID NO: 1026), hsa-miR-4522 (SEQ ID NO: 1027), hsa-miR-34c-3p (SEQ ID NO: 1028), hsa-miR-6784-3p (SEQ ID NO: 1029), hsa-miR-767-3p (SEQ ID NO: 1030), hsa-miR-6771-5p (SEQ ID NO: 1031), hsa-miR-6875-3p (SEQ ID NO: 1032), hsa-miR-1253 (SEQ ID NO: 1033), hsa-miR-4764-5p (SEQ ID NO: 1034), hsa-miR-6789-3p (SEQ ID NO: 1035), hsa-miR-134-3p (SEQ ID NO: 1036), hsa-miR-936 (SEQ ID NO: 1037), hsa-miR-921 (SEQ ID NO: 1038), hsa-miR-193a-3p (SEQ ID NO: 1039), hsa-miR-1185-5p (SEQ ID NO: 1040), hsa-miR-1199-5p (SEQ ID NO: 1041), hsa-miR-6847-5p (SEQ ID NO: 1042), hsa-miR-4655-3p (SEQ ID NO: 1043), hsa-miR-6833-3p (SEQ ID NO: 1044), hsa-miR-1277-3p (SEQ ID NO: 1045), hsa-miR-1538 (SEQ ID NO: 1046), hsa-miR-4776-3p (SEQ ID NO: 1047), hsa-miR-3116 (SEQ ID NO: 1048), hsa-miR-208a-3p (SEQ ID NO: 1049), hsa-miR-30c-2-3p (SEQ ID NO: 1050), hsa-miR-30e-3p (SEQ ID NO: 1051), hsa-miR-149-5p (SEQ ID NO: 1052), hsa-miR-4646-5p (SEQ ID NO: 1053), hsa-miR-3198 (SEQ ID NO: 1054), hsa-miR-9900 (SEQ ID NO: 1055), hsa-miR-221-3p (SEQ ID NO: 1056), hsa-miR-520f-5p (SEQ ID NO: 1057), hsa-miR-3139 (SEQ ID NO: 1058), hsa-miR-4999-5p (SEQ ID NO: 1059), hsa-miR-20b-3p (SEQ ID NO: 1060), hsa-miR-5188 (SEQ ID NO: 1061), hsa-miR-652-3p (SEQ ID NO: 1062), hsa-miR-4737 (SEQ ID NO: 1063), hsa-miR-4734 (SEQ ID NO: 1064), hsa-miR-1469 (SEQ ID NO: 1065), hsa-miR-4490 (SEQ ID NO: 1066), hsa-miR-4260 (SEQ ID NO: 1067), hsa-miR-2115-5p (SEQ ID NO: 1068), hsa-miR-633 (SEQ ID NO: 1069), hsa-miR-4662a-5p (SEQ ID NO: 1070), hsa-miR-5681b (SEQ ID NO: 1071), hsa-miR-6084 (SEQ ID NO: 1072), hsa-miR-4471 (SEQ ID NO: 1073), hsa-miR-585-5p (SEQ ID NO: 1074), hsa-miR-212-3p (SEQ ID NO: 1075), hsa-miR-10396b-5p (SEQ ID NO: 1076), hsa-miR-1224-5p (SEQ ID NO: 1077), hsa-miR-4772-5p (SEQ ID NO: 1078), hsa-miR-6764-3p (SEQ ID NO: 1079), hsa-miR-3158-3p (SEQ ID NO: 1080), hsa-miR-4427 (SEQ ID NO: 1081), hsa-miR-3913-5p (SEQ ID NO: 1082), hsa-miR-553 (SEQ ID NO: 1083), hsa-miR-6740-3p (SEQ ID NO: 1084), hsa-miR-568 (SEQ ID NO: 1085), hsa-miR-3714 (SEQ ID NO: 1086), hsa-miR-592 (SEQ ID NO: 1087), hsa-miR-6741-5p (SEQ ID NO: 1088), hsa-miR-3189-5p (SEQ ID NO: 1089), hsa-miR-3616-5p (SEQ ID NO: 1090), hsa-miR-9983-3p (SEQ ID NO: 1091), hsa-miR-6753-5p (SEQ ID NO: 1092), hsa-miR-520b-5p (SEQ ID NO: 1093), hsa-miR-940 (SEQ ID NO: 1094), hsa-miR-33b-3p (SEQ ID NO: 1095), hsa-miR-588 (SEQ ID NO: 1096), hsa-miR-4668-3p (SEQ ID NO: 1097), hsa-miR-1251-5p (SEQ ID NO: 1098), hsa-miR-8078 (SEQ ID NO: 1099), hsa-miR-5703 (SEQ ID NO: 1100), hsa-miR-146a-3p (SEQ ID NO: 1101), hsa-miR-449c-5p (SEQ ID NO: 1102), hsa-miR-5087 (SEQ ID NO: 1103), hsa-miR-6800-3p (SEQ ID NO: 1104), hsa-miR-11181-3p (SEQ ID NO: 1105), hsa-miR-3913-3p (SEQ ID NO: 1106), hsa-miR-6814-3p (SEQ ID NO: 1107), hsa-miR-1292-3p (SEQ ID NO: 1108), hsa-miR-6735-3p (SEQ ID NO: 1109), hsa-miR-548av-3p (SEQ ID NO: 1110), hsa-miR-5701 (SEQ ID NO: 1111), hsa-miR-450b-3p (SEQ ID NO: 1112), hsa-miR-3187-5p (SEQ ID NO: 1113), hsa-miR-4497 (SEQ ID NO: 1114), hsa-miR-7847-3p (SEQ ID NO: 1115), hsa-miR-6820-5p (SEQ ID NO: 1116), hsa-miR-1181 (SEQ ID NO: 1117), hsa-miR-3121-3p (SEQ ID NO: 1118), hsa-miR-8058 (SEQ ID NO: 1119), hsa-miR-452-5p (SEQ ID NO: 1120), hsa-miR-10397-5p (SEQ ID NO: 1121), hsa-miR-642a-5p (SEQ ID NO: 1122), hsa-miR-1204 (SEQ ID NO: 1123), hsa-miR-12125 (SEQ ID NO: 1124), hsa-miR-6753-3p (SEQ ID NO: 1125), hsa-miR-3156-5p (SEQ ID NO: 1126), hsa-miR-363-5p (SEQ ID NO: 1127), hsa-miR-7851-3p (SEQ ID NO: 1128), hsa-miR-3152-3p (SEQ ID NO: 1129), hsa-miR-607 (SEQ ID NO: 1130), hsa-miR-520c-5p (SEQ ID NO: 1131), hsa-miR-6783-5p (SEQ ID NO: 1132), hsa-miR-580-3p (SEQ ID NO: 1133), hsa-miR-4766-5p (SEQ ID NO: 1134), hsa-miR-4716-3p (SEQ ID NO: 1135), hsa-miR-6881-5p (SEQ ID NO: 1136), hsa-miR-25-5p (SEQ ID NO: 1137), hsa-miR-137-3p (SEQ ID NO: 1138), hsa-miR-3178 (SEQ ID NO: 1139), hsa-miR-548b-3p (SEQ ID NO: 1140), hsa-miR-11401 (SEQ ID NO: 1141), hsa-miR-4651 (SEQ ID NO: 1142), hsa-miR-4798-5p (SEQ ID NO: 1143), hsa-miR-148a-5p (SEQ ID NO: 1144), hsa-miR-4781-3p (SEQ ID NO: 1145), hsa-miR-4538 (SEQ ID NO: 1146), hsa-miR-4716-5p (SEQ ID NO: 1147), hsa-miR-6722-3p (SEQ ID NO: 1148), hsa-miR-3973 (SEQ ID NO: 1149), hsa-miR-99b-5p (SEQ ID NO: 1150), hsa-miR-548au-5p (SEQ ID NO: 1151), hsa-miR-494-3p (SEQ ID NO: 1152), hsa-miR-452-3p (SEQ ID NO: 1153), hsa-miR-19b-2-5p (SEQ ID NO: 1154), hsa-miR-221-5p (SEQ ID NO: 1155), hsa-miR-4322 (SEQ ID NO: 1156), hsa-miR-6780a-3p (SEQ ID NO: 1157), hsa-miR-4329 (SEQ ID NO: 1158), hsa-miR-548at-3p (SEQ ID NO: 1159), hsa-miR-4704-3p (SEQ ID NO: 1160), hsa-miR-3664-3p (SEQ ID NO: 1161), hsa-miR-551a (SEQ ID NO: 1162), hsa-miR-4648 (SEQ ID NO: 1163), hsa-let-7f-2-3p (SEQ ID NO: 1164), hsa-miR-4749-5p (SEQ ID NO: 1165), hsa-miR-7844-5p (SEQ ID NO: 1166), hsa-miR-3167 (SEQ ID NO: 1167), hsa-miR-1295b-5p (SEQ ID NO: 1168), hsa-miR-891a-3p (SEQ ID NO: 1169), hsa-miR-215-5p (SEQ ID NO: 1170), hsa-miR-1292-5p (SEQ ID NO: 1171), hsa-miR-4670-3p (SEQ ID NO: 1172), hsa-miR-605-5p (SEQ ID NO: 1173), hsa-miR-12119 (SEQ ID NO: 1174), hsa-miR-6806-3p (SEQ ID NO: 1175), hsa-miR-4720-5p (SEQ ID NO: 1176), hsa-miR-5684 (SEQ ID NO: 1177), hsa-miR-6758-3p (SEQ ID NO: 1178), hsa-miR-150-5p (SEQ ID NO: 1179), hsa-miR-361-3p (SEQ ID NO: 1180), hsa-miR-4446-3p (SEQ ID NO: 1181), hsa-miR-6133 (SEQ ID NO: 1182), hsa-miR-4281 (SEQ ID NO: 1183), hsa-miR-628-5p (SEQ ID NO: 1184), hsa-miR-100-3p (SEQ ID NO: 1185), hsa-miR-3133 (SEQ ID NO: 1186), hsa-miR-4298 (SEQ ID NO: 1187), hsa-miR-6759-5p (SEQ ID NO: 1188), hsa-miR-6777-3p (SEQ ID NO: 1189), hsa-miR-323a-5p (SEQ ID NO: 1190), hsa-miR-345-5p (SEQ ID NO: 1191), hsa-miR-877-5p (SEQ ID NO: 1192), hsa-miR-5008-5p (SEQ ID NO: 1193), hsa-miR-27b-5p (SEQ ID NO: 1194), hsa-miR-301b-3p (SEQ ID NO: 1195), hsa-miR-5000-3p (SEQ ID NO: 1196), hsa-miR-4776-5p (SEQ ID NO: 1197), hsa-miR-1227-3p (SEQ ID NO: 1198), hsa-miR-659-3p (SEQ ID NO: 1199), hsa-miR-92a-2-5p (SEQ ID NO: 1200), hsa-miR-1256 (SEQ ID NO: 1201), hsa-miR-6813-5p (SEQ ID NO: 1202), hsa-miR-105-5p (SEQ ID NO: 1203), hsa-miR-4755-3p (SEQ ID NO: 1204), hsa-miR-640 (SEQ ID NO: 1205), hsa-miR-199a-3p (SEQ ID NO: 1206), hsa-miR-1224-3p (SEQ ID NO: 1207), hsa-miR-12129 (SEQ ID NO: 1208), hsa-miR-30d-3p (SEQ ID NO: 1209), hsa-miR-1307-5p (SEQ ID NO: 1210), hsa-miR-32-3p (SEQ ID NO: 1211), hsa-miR-4500 (SEQ ID NO: 1212), hsa-miR-525-3p (SEQ ID NO: 1213), hsa-miR-708-5p (SEQ ID NO: 1214), hsa-miR-4273 (SEQ ID NO: 1215), hsa-miR-488-3p (SEQ ID NO: 1216), hsa-miR-4466 (SEQ ID NO: 1217), hsa-miR-2467-3p (SEQ ID NO: 1218), hsa-miR-6764-5p (SEQ ID NO: 1219), hsa-miR-548y (SEQ ID NO: 1220), hsa-miR-3928-5p (SEQ ID NO: 1221), hsa-miR-3914 (SEQ ID NO: 1222), hsa-miR-34a-5p (SEQ ID NO: 1223), hsa-miR-711 (SEQ ID NO: 1224), hsa-miR-335-5p (SEQ ID NO: 1225), hsa-miR-5588-3p (SEQ ID NO: 1226), hsa-miR-6769a-5p (SEQ ID NO: 1227), hsa-miR-99b-3p (SEQ ID NO: 1228), hsa-miR-5091 (SEQ ID NO: 1229), hsa-miR-6848-3p (SEQ ID NO: 1230), hsa-miR-26a-5p (SEQ ID NO: 1231), hsa-miR-4793-5p (SEQ ID NO: 1232), hsa-miR-6819-5p (SEQ ID NO: 1233), hsa-miR-6747-3p (SEQ ID NO: 1234), hsa-miR-190a-5p (SEQ ID NO: 1235), hsa-miR-6728-3p (SEQ ID NO: 1236), hsa-miR-7109-5p (SEQ ID NO: 1237), hsa-miR-3674 (SEQ ID NO: 1238), hsa-miR-4647 (SEQ ID NO: 1239), hsa-miR-3165 (SEQ ID NO: 1240), hsa-miR-548c-3p (SEQ ID NO: 1241), hsa-miR-4308 (SEQ ID NO: 1242), hsa-miR-6731-5p (SEQ ID NO: 1243), hsa-miR-3130-3p (SEQ ID NO: 1244), hsa-miR-3666 (SEQ ID NO: 1245), hsa-miR-122b-5p (SEQ ID NO: 1246), hsa-miR-4639-5p (SEQ ID NO: 1247), hsa-miR-297 (SEQ ID NO: 1248), hsa-miR-1264 (SEQ ID NO: 1249), hsa-miR-5583-3p (SEQ ID NO: 1250), hsa-miR-3188 (SEQ ID NO: 1251), hsa-let-7a-5p (SEQ ID NO: 1252), hsa-miR-6080 (SEQ ID NO: 1253), hsa-miR-7-5p (SEQ ID NO: 1254), hsa-miR-3136-5p (SEQ ID NO: 1255), hsa-miR-499a-3p (SEQ ID NO: 1256), hsa-miR-629-3p (SEQ ID NO: 1257), hsa-miR-4771 (SEQ ID NO: 1258), hsa-miR-518d-3p (SEQ ID NO: 1259), hsa-miR-6815-5p (SEQ ID NO: 1260), hsa-miR-6746-5p (SEQ ID NO: 1261), hsa-miR-6772-3p (SEQ ID NO: 1262), hsa-miR-874-3p (SEQ ID NO: 1263), hsa-miR-130a-5p (SEQ ID NO: 1264), hsa-miR-331-3p (SEQ ID NO: 1265), hsa-miR-187-3p (SEQ ID NO: 1266), hsa-miR-3150a-3p (SEQ ID NO: 1267), hsa-miR-4433b-3p (SEQ ID NO: 1268), hsa-miR-924 (SEQ ID NO: 1269), hsa-miR-523-3p (SEQ ID NO: 1270), hsa-miR-579-3p (SEQ ID NO: 1271), hsa-miR-577 (SEQ ID NO: 1272), hsa-miR-382-5p (SEQ ID NO: 1273), hsa-miR-4770 (SEQ ID NO: 1274), hsa-miR-8083 (SEQ ID NO: 1275), hsa-miR-218-1-3p (SEQ ID NO: 1276), hsa-miR-3115 (SEQ ID NO: 1277), hsa-miR-6877-3p (SEQ ID NO: 1278), hsa-miR-4746-5p (SEQ ID NO: 1279), hsa-miR-5010-3p (SEQ ID NO: 1280), hsa-miR-376a-5p (SEQ ID NO: 1281), hsa-miR-188-5p (SEQ ID NO: 1282), hsa-miR-7978 (SEQ ID NO: 1283), hsa-miR-495-3p (SEQ ID NO: 1284), hsa-miR-4507 (SEQ ID NO: 1285), hsa-miR-4293 (SEQ ID NO: 1286), hsa-miR-4516 (SEQ ID NO: 1287), hsa-miR-4475 (SEQ ID NO: 1288), hsa-miR-1910-3p (SEQ ID NO: 1289), hsa-miR-4681 (SEQ ID NO: 1290), hsa-miR-152-5p (SEQ ID NO: 1291), hsa-miR-409-3p (SEQ ID NO: 1292), hsa-miR-204-5p (SEQ ID NO: 1293), hsa-miR-1249-3p (SEQ ID NO: 1294), hsa-miR-4787-3p (SEQ ID NO: 1295), hsa-miR-6071 (SEQ ID NO: 1296), hsa-miR-3920 (SEQ ID NO: 1297), hsa-miR-548az-3p (SEQ ID NO: 1298), hsa-miR-6879-5p (SEQ ID NO: 1299), hsa-miR-183-5p (SEQ ID NO: 1300), hsa-miR-6878-3p (SEQ ID NO: 1301), hsa-miR-544a (SEQ ID NO: 1302), hsa-miR-135b-3p (SEQ ID NO: 1303), hsa-miR-7973 (SEQ ID NO: 1304), hsa-miR-548ai (SEQ ID NO: 1305), hsa-miR-501-3p (SEQ ID NO: 1306), hsa-miR-4695-5p (SEQ ID NO: 1307), hsa-miR-1251-3p (SEQ ID NO: 1308), hsa-miR-548ao-5p (SEQ ID NO: 1309), hsa-miR-4692 (SEQ ID NO: 1310), hsa-miR-103b (SEQ ID NO: 1311), hsa-miR-3665 (SEQ ID NO: 1312), hsa-miR-524-3p (SEQ ID NO: 1313), hsa-miR-214-3p (SEQ ID NO: 1314), hsa-miR-1197 (SEQ ID NO: 1315), hsa-miR-3141 (SEQ ID NO: 1316), hsa-miR-346 (SEQ ID NO: 1317), hsa-miR-21-5p (SEQ ID NO: 1318), hsa-miR-6885-3p (SEQ ID NO: 1319), hsa-miR-197-3p (SEQ ID NO: 1320), hsa-miR-12113 (SEQ ID NO: 1321), hsa-miR-4786-5p (SEQ ID NO: 1322), hsa-miR-205-3p (SEQ ID NO: 1323), hsa-miR-5582-3p (SEQ ID NO: 1324), hsa-miR-4485-5p (SEQ ID NO: 1325), hsa-miR-513c-5p (SEQ ID NO: 1326), hsa-miR-8066 (SEQ ID NO: 1327), hsa-miR-145-5p (SEQ ID NO: 1328), hsa-miR-5693 (SEQ ID NO: 1329), hsa-miR-4780 (SEQ ID NO: 1330), hsa-miR-4801 (SEQ ID NO: 1331), hsa-miR-602 (SEQ ID NO: 1332), hsa-miR-3935 (SEQ ID NO: 1333), hsa-miR-2355-5p (SEQ ID NO: 1334), hsa-miR-4252 (SEQ ID NO: 1335), hsa-miR-8065 (SEQ ID NO: 1336), hsa-miR-668-5p (SEQ ID NO: 1337), hsa-miR-4768-3p (SEQ ID NO: 1338), hsa-miR-552-5p (SEQ ID NO: 1339), hsa-miR-5582-5p (SEQ ID NO: 1340), hsa-miR-6124 (SEQ ID NO: 1341), hsa-miR-4453 (SEQ ID NO: 1342), hsa-miR-3689b-3p (SEQ ID NO: 1343), hsa-miR-10401-5p (SEQ ID NO: 1344), hsa-miR-433-3p (SEQ ID NO: 1345), hsa-miR-187-5p (SEQ ID NO: 1346), hsa-miR-613 (SEQ ID NO: 1347), hsa-miR-1293 (SEQ ID NO: 1348), hsa-miR-6830-5p (SEQ ID NO: 1349), hsa-miR-1225-3p (SEQ ID NO: 1350), hsa-miR-4749-3p (SEQ ID NO: 1351), hsa-miR-4643 (SEQ ID NO: 1352), hsa-miR-210-3p (SEQ ID NO: 1353), hsa-miR-302d-3p (SEQ ID NO: 1354), hsa-miR-6850-3p (SEQ ID NO: 1355), hsa-miR-6762-5p (SEQ ID NO: 1356), hsa-miR-3177-3p (SEQ ID NO: 1357), hsa-miR-4687-5p (SEQ ID NO: 1358), hsa-miR-2682-5p (SEQ ID NO: 1359), hsa-miR-520f-3p (SEQ ID NO: 1360), hsa-miR-656-5p (SEQ ID NO: 1361), hsa-miR-5197-3p (SEQ ID NO: 1362), hsa-miR-487a-3p (SEQ ID NO: 1363), hsa-miR-30d-5p (SEQ ID NO: 1364), hsa-miR-548av-5p (SEQ ID NO: 1365), hsa-miR-6827-3p (SEQ ID NO: 1366), hsa-miR-518d-5p (SEQ ID NO: 1367), hsa-miR-4740-5p (SEQ ID NO: 1368), hsa-miR-637 (SEQ ID NO: 1369), hsa-miR-7108-5p (SEQ ID NO: 1370), hsa-miR-6887-5p (SEQ ID NO: 1371), hsa-miR-3690 (SEQ ID NO: 1372), hsa-miR-34b-5p (SEQ ID NO: 1373), hsa-miR-6786-3p (SEQ ID NO: 1374), hsa-miR-518a-3p (SEQ ID NO: 1375), hsa-miR-3975 (SEQ ID NO: 1376), hsa-miR-645 (SEQ ID NO: 1377), hsa-miR-4524b-3p (SEQ ID NO: 1378), hsa-miR-17-5p (SEQ ID NO: 1379), hsa-miR-6895-3p (SEQ ID NO: 1380), hsa-miR-6873-5p (SEQ ID NO: 1381), hsa-miR-4674 (SEQ ID NO: 1382), hsa-miR-3200-5p (SEQ ID NO: 1383), hsa-miR-2909 (SEQ ID NO: 1384), hsa-miR-4730 (SEQ ID NO: 1385), hsa-miR-3681-5p (SEQ ID NO: 1386), hsa-miR-6876-5p (SEQ ID NO: 1387), hsa-miR-302b-3p (SEQ ID NO: 1388), hsa-miR-92b-3p (SEQ ID NO: 1389), hsa-miR-3140-5p (SEQ ID NO: 1390), hsa-miR-3680-5p (SEQ ID NO: 1391), hsa-miR-8069 (SEQ ID NO: 1392), hsa-miR-526a-3p (SEQ ID NO: 1393), hsa-miR-6506-3p (SEQ ID NO: 1394), hsa-miR-3187-3p (SEQ ID NO: 1395), hsa-miR-758-5p (SEQ ID NO: 1396), hsa-miR-5089-3p (SEQ ID NO: 1397), hsa-miR-3977 (SEQ ID NO: 1398), hsa-miR-5682 (SEQ ID NO: 1399), hsa-miR-4695-3p (SEQ ID NO: 1400), hsa-miR-377-5p (SEQ ID NO: 1401), hsa-miR-562 (SEQ ID NO: 1402), hsa-miR-4328 (SEQ ID NO: 1403), hsa-miR-202-3p (SEQ ID NO: 1404), hsa-miR-5196-3p (SEQ ID NO: 1405), hsa-miR-4314 (SEQ ID NO: 1406), hsa-miR-3681-3p (SEQ ID NO: 1407), hsa-miR-2114-3p (SEQ ID NO: 1408), hsa-miR-5692a (SEQ ID NO: 1409), hsa-miR-615-5p (SEQ ID NO: 1410), hsa-miR-3677-3p (SEQ ID NO: 1411), hsa-miR-138-1-3p (SEQ ID NO: 1412), hsa-miR-4641 (SEQ ID NO: 1413), hsa-miR-370-5p (SEQ ID NO: 1414), hsa-miR-4732-5p (SEQ ID NO: 1415), hsa-miR-6760-3p (SEQ ID NO: 1416), hsa-miR-7162-5p (SEQ ID NO: 1417), hsa-miR-8060 (SEQ ID NO: 1418), hsa-miR-1237-5p (SEQ ID NO: 1419), hsa-miR-4421 (SEQ ID NO: 1420), hsa-miR-3195 (SEQ ID NO: 1421), hsa-miR-193a-5p (SEQ ID NO: 1422), hsa-miR-4486 (SEQ ID NO: 1423), hsa-miR-4534 (SEQ ID NO: 1424), hsa-miR-2278 (SEQ ID NO: 1425), hsa-miR-892c-5p (SEQ ID NO: 1426), hsa-miR-1179 (SEQ ID NO: 1427), hsa-miR-6834-3p (SEQ ID NO: 1428), hsa-miR-380-5p (SEQ ID NO: 1429), hsa-miR-19b-1-5p (SEQ ID NO: 1430), hsa-miR-6759-3p (SEQ ID NO: 1431), hsa-miR-500a-5p (SEQ ID NO: 1432), hsa-miR-744-5p (SEQ ID NO: 1433), hsa-miR-26a-2-3p (SEQ ID NO: 1434), hsa-miR-3605-3p (SEQ ID NO: 1435), hsa-miR-4677-3p (SEQ ID NO: 1436), hsa-miR-34a-3p (SEQ ID NO: 1437), hsa-miR-3122 (SEQ ID NO: 1438), hsa-miR-758-3p (SEQ ID NO: 1439), hsa-miR-4274 (SEQ ID NO: 1440), hsa-miR-6732-5p (SEQ ID NO: 1441), hsa-miR-598-5p (SEQ ID NO: 1442), hsa-miR-4465 (SEQ ID NO: 1443), hsa-miR-548ar-3p (SEQ ID NO: 1444), hsa-miR-4690-3p (SEQ ID NO: 1445), hsa-miR-202-5p (SEQ ID NO: 1446), hsa-miR-378d (SEQ ID NO: 1447), hsa-miR-6765-5p (SEQ ID NO: 1448), hsa-miR-6511a-5p (SEQ ID NO: 1449), hsa-miR-1267 (SEQ ID NO: 1450), hsa-miR-4668-5p (SEQ ID NO: 1451), hsa-miR-6730-3p (SEQ ID NO: 1452), hsa-miR-4671-5p (SEQ ID NO: 1453), hsa-miR-920 (SEQ ID NO: 1454), hsa-miR-6826-5p (SEQ ID NO: 1455), hsa-miR-5092 (SEQ ID NO: 1456), hsa-miR-548b-5p (SEQ ID NO: 1457), hsa-miR-135a-3p (SEQ ID NO: 1458), hsa-miR-516a-3p (SEQ ID NO: 1459), hsa-miR-4513 (SEQ ID NO: 1460), hsa-miR-518e-5p (SEQ ID NO: 1461), hsa-miR-205-5p (SEQ ID NO: 1462), hsa-miR-593-3p (SEQ ID NO: 1463), hsa-miR-6836-5p (SEQ ID NO: 1464), hsa-miR-6515-5p (SEQ ID NO: 1465), hsa-miR-653-5p (SEQ ID NO: 1466), hsa-miR-6503-5p (SEQ ID NO: 1467), hsa-miR-6504-5p (SEQ ID NO: 1468), hsa-miR-627-5p (SEQ ID NO: 1469), hsa-miR-3618 (SEQ ID NO: 1470), hsa-miR-5186 (SEQ ID NO: 1471), hsa-miR-513a-3p (SEQ ID NO: 1472), hsa-miR-10392-3p (SEQ ID NO: 1473), hsa-miR-210-5p (SEQ ID NO: 1474), hsa-miR-5196-5p (SEQ ID NO: 1475), hsa-miR-4708-3p (SEQ ID NO: 1476), hsa-miR-6078 (SEQ ID NO: 1477), hsa-miR-3173-5p (SEQ ID NO: 1478), hsa-miR-4748 (SEQ ID NO: 1479), hsa-miR-4302 (SEQ ID NO: 1480), hsa-miR-4735-3p (SEQ ID NO: 1481), hsa-miR-223-3p (SEQ ID NO: 1482), hsa-miR-142-5p (SEQ ID NO: 1483), hsa-miR-6803-3p (SEQ ID NO: 1484), hsa-miR-6511a-3p (SEQ ID NO: 1485), hsa-miR-5583-5p (SEQ ID NO: 1486), hsa-miR-507 (SEQ ID NO: 1487), hsa-miR-3171 (SEQ ID NO: 1488), hsa-miR-3908 (SEQ ID NO: 1489), hsa-miR-4761-5p (SEQ ID NO: 1490), hsa-miR-8053 (SEQ ID NO: 1491), hsa-miR-186-3p (SEQ ID NO: 1492), hsa-miR-3689d (SEQ ID NO: 1493), hsa-miR-6769b-3p (SEQ ID NO: 1494), hsa-miR-12115 (SEQ ID NO: 1495), hsa-miR-7106-3p (SEQ ID NO: 1496), hsa-miR-10a-3p (SEQ ID NO: 1497), hsa-miR-6815-3p (SEQ ID NO: 1498), hsa-miR-6088 (SEQ ID NO: 1499), hsa-miR-1-3p (SEQ ID NO: 1500), hsa-miR-200a-5p (SEQ ID NO: 1501), hsa-miR-3202 (SEQ ID NO: 1502), hsa-miR-943 (SEQ ID NO: 1503), hsa-miR-6833-5p (SEQ ID NO: 1504), hsa-miR-6883-5p (SEQ ID NO: 1505), hsa-miR-6890-3p (SEQ ID NO: 1506), hsa-miR-3149 (SEQ ID NO: 1507), hsa-let-7b-3p (SEQ ID NO: 1508), hsa-miR-6878-5p (SEQ ID NO: 1509), hsa-miR-146b-3p (SEQ ID NO: 1510), hsa-miR-10399-3p (SEQ ID NO: 1511), hsa-miR-4731-5p (SEQ ID NO: 1512), hsa-let-7g-3p (SEQ ID NO: 1513), hsa-miR-5590-5p (SEQ ID NO: 1514), hsa-miR-551b-5p (SEQ ID NO: 1515), hsa-miR-3186-3p (SEQ ID NO: 1516), hsa-miR-302a-5p (SEQ ID NO: 1517), hsa-miR-769-5p (SEQ ID NO: 1518), hsa-miR-19a-3p (SEQ ID NO: 1519), hsa-miR-381-5p (SEQ ID NO: 1520), hsa-miR-6741-3p (SEQ ID NO: 1521), hsa-miR-6845-5p (SEQ ID NO: 1522), hsa-miR-7155-5p (SEQ ID NO: 1523), hsa-miR-544b (SEQ ID NO: 1524), hsa-miR-4505 (SEQ ID NO: 1525), hsa-miR-8073 (SEQ ID NO: 1526), hsa-miR-4633-3p (SEQ ID NO: 1527), hsa-miR-4694-5p (SEQ ID NO: 1528), hsa-miR-3677-5p (SEQ ID NO: 1529), hsa-miR-4491 (SEQ ID NO: 1530), hsa-miR-7848-3p (SEQ ID NO: 1531), hsa-miR-668-3p (SEQ ID NO: 1532), hsa-miR-938 (SEQ ID NO: 1533), hsa-miR-3074-3p (SEQ ID NO: 1534), hsa-miR-6716-3p (SEQ ID NO: 1535), hsa-miR-342-3p (SEQ ID NO: 1536), hsa-miR-5587-5p (SEQ ID NO: 1537), hsa-miR-584-5p (SEQ ID NO: 1538), hsa-miR-298 (SEQ ID NO: 1539), hsa-miR-8055 (SEQ ID NO: 1540), hsa-miR-3612 (SEQ ID NO: 1541), hsa-miR-542-5p (SEQ ID NO: 1542), hsa-miR-19b-3p (SEQ ID NO: 1543), hsa-miR-1231 (SEQ ID NO: 1544), hsa-miR-4255 (SEQ ID NO: 1545), hsa-miR-10396a-3p (SEQ ID NO: 1546), hsa-miR-675-5p (SEQ ID NO: 1547), hsa-miR-199a-5p (SEQ ID NO: 1548), hsa-miR-513b-5p (SEQ ID NO: 1549), hsa-miR-4455 (SEQ ID NO: 1550), hsa-miR-3682-3p (SEQ ID NO: 1551), hsa-miR-9500 (SEQ ID NO: 1552), hsa-miR-6828-5p (SEQ ID NO: 1553), hsa-miR-6718-5p (SEQ ID NO: 1554), hsa-miR-939-3p (SEQ ID NO: 1555), hsa-miR-548ad-5p (SEQ ID NO: 1556), hsa-miR-339-3p (SEQ ID NO: 1557), hsa-miR-4739 (SEQ ID NO: 1558), hsa-miR-630 (SEQ ID NO: 1559), hsa-miR-1246 (SEQ ID NO: 1560), hsa-miR-3135b (SEQ ID NO: 1561), hsa-miR-600 (SEQ ID NO: 1562), hsa-miR-129-1-3p (SEQ ID NO: 1563), hsa-miR-4268 (SEQ ID NO: 1564), hsa-miR-876-5p (SEQ ID NO: 1565), hsa-miR-3145-5p (SEQ ID NO: 1566), hsa-miR-12122 (SEQ ID NO: 1567), hsa-miR-582-5p (SEQ ID NO: 1568), hsa-miR-3085-3p (SEQ ID NO: 1569), hsa-miR-1470 (SEQ ID NO: 1570), hsa-miR-4291 (SEQ ID NO: 1571), hsa-miR-766-5p (SEQ ID NO: 1572), hsa-miR-548ae-5p (SEQ ID NO: 1573), hsa-miR-1271-3p (SEQ ID NO: 1574), hsa-miR-200c-3p (SEQ ID NO: 1575), hsa-miR-4721 (SEQ ID NO: 1576), hsa-miR-6810-5p (SEQ ID NO: 1577), hsa-miR-6853-5p (SEQ ID NO: 1578), hsa-miR-4744 (SEQ ID NO: 1579), hsa-miR-3670 (SEQ ID NO: 1580), hsa-miR-3925-3p (SEQ ID NO: 1581), hsa-miR-17-3p (SEQ ID NO: 1582), hsa-miR-5585-5p (SEQ ID NO: 1583), hsa-miR-4652-3p (SEQ ID NO: 1584), hsa-miR-196b-3p (SEQ ID NO: 1585), hsa-miR-10523-5p (SEQ ID NO: 1586), hsa-miR-3200-3p (SEQ ID NO: 1587), hsa-miR-196a-3p (SEQ ID NO: 1588), hsa-miR-320e (SEQ ID NO: 1589), hsa-miR-4804-3p (SEQ ID NO: 1590), hsa-miR-4433a-5p (SEQ ID NO: 1591), hsa-miR-3689a-3p (SEQ ID NO: 1592), hsa-miR-548ba (SEQ ID NO: 1593), hsa-miR-548d-3p (SEQ ID NO: 1594), hsa-miR-10393-3p (SEQ ID NO: 1595), hsa-miR-8080 (SEQ ID NO: 1596), hsa-miR-4316 (SEQ ID NO: 1597), hsa-miR-548az-5p (SEQ ID NO: 1598), hsa-miR-4717-5p (SEQ ID NO: 1599), hsa-miR-10400-3p (SEQ ID NO: 1600), hsa-miR-4690-5p (SEQ ID NO: 1601), hsa-miR-3678-3p (SEQ ID NO: 1602), hsa-miR-3194-5p (SEQ ID NO: 1603), hsa-miR-605-3p (SEQ ID NO: 1604), hsa-miR-4706 (SEQ ID NO: 1605), hsa-miR-4725-3p (SEQ ID NO: 1606), hsa-miR-4432 (SEQ ID NO: 1607), hsa-miR-7849-3p (SEQ ID NO: 1608), hsa-miR-503-3p (SEQ ID NO: 1609), hsa-miR-1307-3p (SEQ ID NO: 1610), hsa-miR-6731-3p (SEQ ID NO: 1611), hsa-miR-376a-3p (SEQ ID NO: 1612), hsa-miR-6859-3p (SEQ ID NO: 1613), hsa-miR-5000-5p (SEQ ID NO: 1614), hsa-miR-3065-5p (SEQ ID NO: 1615), hsa-miR-6822-3p (SEQ ID NO: 1616), hsa-miR-8086 (SEQ ID NO: 1617), hsa-miR-217-3p (SEQ ID NO: 1618), hsa-miR-125b-2-3p (SEQ ID NO: 1619), hsa-miR-548am-5p (SEQ ID NO: 1620), hsa-miR-323b-3p (SEQ ID NO: 1621), hsa-miR-4489 (SEQ ID NO: 1622), hsa-miR-6862-3p (SEQ ID NO: 1623), hsa-miR-194-3p (SEQ ID NO: 1624), hsa-miR-6772-5p (SEQ ID NO: 1625), hsa-miR-223-5p (SEQ ID NO: 1626), hsa-miR-569 (SEQ ID NO: 1627), hsa-miR-520b-3p (SEQ ID NO: 1628), hsa-miR-4680-5p (SEQ ID NO: 1629), hsa-miR-1914-3p (SEQ ID NO: 1630), hsa-miR-4740-3p (SEQ ID NO: 1631), hsa-miR-518b (SEQ ID NO: 1632), hsa-miR-504-3p (SEQ ID NO: 1633), hsa-miR-149-3p (SEQ ID NO: 1634), hsa-miR-4758-5p (SEQ ID NO: 1635), hsa-miR-26b-5p (SEQ ID NO: 1636), hsa-miR-5708 (SEQ ID NO: 1637), hsa-miR-1236-3p (SEQ ID NO: 1638), hsa-miR-670-3p (SEQ ID NO: 1639), hsa-miR-573 (SEQ ID NO: 1640), hsa-miR-3912-5p (SEQ ID NO: 1641), hsa-miR-12130 (SEQ ID NO: 1642), hsa-miR-4783-5p (SEQ ID NO: 1643), hsa-miR-3686 (SEQ ID NO: 1644), hsa-miR-660-3p (SEQ ID NO: 1645), hsa-miR-3655 (SEQ ID NO: 1646), hsa-miR-4720-3p (SEQ ID NO: 1647), hsa-miR-3160-5p (SEQ ID NO: 1648), hsa-miR-1202 (SEQ ID NO: 1649), hsa-miR-765 (SEQ ID NO: 1650), hsa-miR-628-3p (SEQ ID NO: 1651), hsa-miR-12135 (SEQ ID NO: 1652), hsa-miR-532-5p (SEQ ID NO: 1653), hsa-miR-3131 (SEQ ID NO: 1654), hsa-miR-296-5p (SEQ ID NO: 1655), hsa-miR-378a-5p (SEQ ID NO: 1656), hsa-miR-6769a-3p (SEQ ID NO: 1657), hsa-miR-6802-5p (SEQ ID NO: 1658), hsa-miR-1911-5p (SEQ ID NO: 1659), hsa-miR-20a-5p (SEQ ID NO: 1660), hsa-miR-6128 (SEQ ID NO: 1661), hsa-miR-5586-5p (SEQ ID NO: 1662), hsa-miR-4790-5p (SEQ ID NO: 1663), hsa-miR-188-3p (SEQ ID NO: 1664), hsa-miR-4654 (SEQ ID NO: 1665), hsa-miR-21-3p (SEQ ID NO: 1666), hsa-miR-6859-5p (SEQ ID NO: 1667), hsa-miR-550b-2-5p (SEQ ID NO: 1668), hsa-miR-4527 (SEQ ID NO: 1669), hsa-miR-6782-5p (SEQ ID NO: 1670), hsa-miR-2355-3p (SEQ ID NO: 1671), hsa-miR-2117 (SEQ ID NO: 1672), hsa-miR-148b-3p (SEQ ID NO: 1673), hsa-miR-6864-3p (SEQ ID NO: 1674), hsa-miR-590-3p (SEQ ID NO: 1675), hsa-miR-5589-5p (SEQ ID NO: 1676), hsa-miR-100-5p (SEQ ID NO: 1677), hsa-miR-548u (SEQ ID NO: 1678), hsa-miR-6837-3p (SEQ ID NO: 1679), hsa-miR-6774-5p (SEQ ID NO: 1680), hsa-miR-4263 (SEQ ID NO: 1681), hsa-miR-320a-3p (SEQ ID NO: 1682), hsa-miR-8074 (SEQ ID NO: 1683), hsa-miR-4666a-5p (SEQ ID NO: 1684), hsa-miR-6808-3p (SEQ ID NO: 1685), hsa-miR-6743-3p (SEQ ID NO: 1686), hsa-miR-511-5p (SEQ ID NO: 1687), hsa-miR-4632-5p (SEQ ID NO: 1688), hsa-miR-301a-3p (SEQ ID NO: 1689), hsa-miR-1200 (SEQ ID NO: 1690), hsa-miR-3199 (SEQ ID NO: 1691), hsa-miR-152-3p (SEQ ID NO: 1692), hsa-miR-361-5p (SEQ ID NO: 1693), hsa-miR-1199-3p (SEQ ID NO: 1694), hsa-miR-6751-3p (SEQ ID NO: 1695), hsa-miR-6757-3p (SEQ ID NO: 1696), hsa-miR-6871-5p (SEQ ID NO: 1697), hsa-miR-656-3p (SEQ ID NO: 1698), hsa-miR-4728-5p (SEQ ID NO: 1699), hsa-miR-23c (SEQ ID NO: 1700), hsa-miR-4717-3p (SEQ ID NO: 1701), hsa-miR-517c-3p (SEQ ID NO: 1702), hsa-miR-520c-3p (SEQ ID NO: 1703), hsa-miR-6854-5p (SEQ ID NO: 1704), hsa-miR-4310 (SEQ ID NO: 1705), hsa-miR-6849-3p (SEQ ID NO: 1706), hsa-miR-5591-5p (SEQ ID NO: 1707), hsa-miR-4277 (SEQ ID NO: 1708), hsa-miR-509-3p (SEQ ID NO: 1709), hsa-miR-4722-3p (SEQ ID NO: 1710), hsa-miR-6729-5p (SEQ ID NO: 1711), hsa-miR-4496 (SEQ ID NO: 1712), hsa-miR-4764-3p (SEQ ID NO: 1713), hsa-miR-4782-5p (SEQ ID NO: 1714), hsa-miR-1323 (SEQ ID NO: 1715), hsa-miR-708-3p (SEQ ID NO: 1716), hsa-let-7i-5p (SEQ ID NO: 1717), hsa-miR-384 (SEQ ID NO: 1718), hsa-miR-4292 (SEQ ID NO: 1719), hsa-miR-4699-3p (SEQ ID NO: 1720), hsa-miR-1252-5p (SEQ ID NO: 1721), hsa-miR-500b-3p (SEQ ID NO: 1722), hsa-miR-4802-5p (SEQ ID NO: 1723), hsa-miR-4509 (SEQ ID NO: 1724), hsa-miR-6090 (SEQ ID NO: 1725), hsa-miR-654-3p (SEQ ID NO: 1726), hsa-miR-4679 (SEQ ID NO: 1727), hsa-miR-429 (SEQ ID NO: 1728), hsa-miR-584-3p (SEQ ID NO: 1729), hsa-miR-4655-5p (SEQ ID NO: 1730), hsa-miR-508-3p (SEQ ID NO: 1731), hsa-miR-4762-5p (SEQ ID NO: 1732), hsa-miR-6808-5p (SEQ ID NO: 1733), hsa-miR-6837-5p (SEQ ID NO: 1734), hsa-miR-200b-5p (SEQ ID NO: 1735), hsa-miR-6801-3p (SEQ ID NO: 1736), hsa-miR-548d-5p (SEQ ID NO: 1737), hsa-miR-3157-3p (SEQ ID NO: 1738), hsa-miR-3059-5p (SEQ ID NO: 1739), hsa-miR-324-5p (SEQ ID NO: 1740), hsa-miR-3713 (SEQ ID NO: 1741), hsa-miR-4280 (SEQ ID NO: 1742), hsa-miR-26a-1-3p (SEQ ID NO: 1743), hsa-miR-6743-5p (SEQ ID NO: 1744), hsa-miR-6855-5p (SEQ ID NO: 1745), hsa-miR-1294 (SEQ ID NO: 1746), hsa-miR-5696 (SEQ ID NO: 1747), hsa-miR-548aa (SEQ ID NO: 1748), hsa-miR-7158-5p (SEQ ID NO: 1749), hsa-miR-3157-5p (SEQ ID NO: 1750), hsa-miR-154-3p (SEQ ID NO: 1751), hsa-miR-122-3p (SEQ ID NO: 1752), hsa-miR-1270 (SEQ ID NO: 1753), hsa-miR-769-3p (SEQ ID NO: 1754), hsa-miR-3168 (SEQ ID NO: 1755), hsa-miR-143-3p (SEQ ID NO: 1756), hsa-miR-6874-5p (SEQ ID NO: 1757), hsa-miR-516b-3p (SEQ ID NO: 1758), hsa-miR-4677-5p (SEQ ID NO: 1759), hsa-miR-10b-5p (SEQ ID NO: 1760), hsa-miR-7705 (SEQ ID NO: 1761), hsa-miR-6755-5p (SEQ ID NO: 1762), hsa-miR-6847-3p (SEQ ID NO: 1763), hsa-miR-512-3p (SEQ ID NO: 1764), hsa-miR-153-3p (SEQ ID NO: 1765), hsa-miR-4454 (SEQ ID NO: 1766), hsa-miR-8064 (SEQ ID NO: 1767), hsa-miR-7110-3p (SEQ ID NO: 1768), hsa-miR-423-3p (SEQ ID NO: 1769), hsa-miR-331-5p (SEQ ID NO: 1770), hsa-miR-190a-3p (SEQ ID NO: 1771), hsa-miR-610 (SEQ ID NO: 1772), hsa-miR-198 (SEQ ID NO: 1773), hsa-miR-4536-5p (SEQ ID NO: 1774), hsa-miR-4760-5p (SEQ ID NO: 1775), hsa-miR-3924 (SEQ ID NO: 1776), hsa-miR-5692b (SEQ ID NO: 1777), hsa-miR-6742-5p (SEQ ID NO: 1778), hsa-miR-935 (SEQ ID NO: 1779), hsa-miR-612 (SEQ ID NO: 1780), hsa-miR-8089 (SEQ ID NO: 1781), hsa-miR-6774-3p (SEQ ID NO: 1782), hsa-miR-4503 (SEQ ID NO: 1783), hsa-miR-762 (SEQ ID NO: 1784), hsa-miR-150-3p (SEQ ID NO: 1785), hsa-miR-4999-3p (SEQ ID NO: 1786), hsa-miR-548aq-5p (SEQ ID NO: 1787), hsa-miR-6883-3p (SEQ ID NO: 1788), hsa-miR-497-3p (SEQ ID NO: 1789), hsa-miR-3689c (SEQ ID NO: 1790), hsa-miR-548bb-3p (SEQ ID NO: 1791), hsa-miR-8082 (SEQ ID NO: 1792), hsa-miR-4315 (SEQ ID NO: 1793), hsa-miR-6818-5p (SEQ ID NO: 1794), hsa-miR-6506-5p (SEQ ID NO: 1795), hsa-miR-676-3p (SEQ ID NO: 1796), hsa-miR-6508-3p (SEQ ID NO: 1797), hsa-miR-3181 (SEQ ID NO: 1798), hsa-miR-3927-3p (SEQ ID NO: 1799), hsa-miR-5193 (SEQ ID NO: 1800), hsa-miR-376b-3p (SEQ ID NO: 1801), hsa-miR-7843-5p (SEQ ID NO: 1802), hsa-miR-6781-3p (SEQ ID NO: 1803), hsa-miR-4259 (SEQ ID NO: 1804), hsa-miR-371a-5p (SEQ ID NO: 1805), hsa-miR-548e-5p (SEQ ID NO: 1806), hsa-miR-651-5p (SEQ ID NO: 1807), hsa-miR-10397-3p (SEQ ID NO: 1808), hsa-miR-422a (SEQ ID NO: 1809), hsa-miR-4642 (SEQ ID NO: 1810), hsa-miR-4724-3p (SEQ ID NO: 1811), hsa-miR-7160-3p (SEQ ID NO: 1812), hsa-miR-6744-5p (SEQ ID NO: 1813), hsa-miR-4524a-3p (SEQ ID NO: 1814), hsa-miR-873-3p (SEQ ID NO: 1815), hsa-miR-362-3p (SEQ ID NO: 1816), hsa-miR-4758-3p (SEQ ID NO: 1817), hsa-miR-1263 (SEQ ID NO: 1818), hsa-miR-6512-3p (SEQ ID NO: 1819), hsa-miR-3911 (SEQ ID NO: 1820), hsa-miR-6073 (SEQ ID NO: 1821), hsa-miR-1257 (SEQ ID NO: 1822), hsa-miR-670-5p (SEQ ID NO: 1823), hsa-miR-4429 (SEQ ID NO: 1824), hsa-miR-4711-5p (SEQ ID NO: 1825), hsa-miR-3646 (SEQ ID NO: 1826), hsa-miR-4667-5p (SEQ ID NO: 1827), hsa-miR-6085 (SEQ ID NO: 1828), hsa-miR-146b-5p (SEQ ID NO: 1829), hsa-miR-6730-5p (SEQ ID NO: 1830), hsa-miR-378a-3p (SEQ ID NO: 1831), hsa-miR-3650 (SEQ ID NO: 1832), hsa-miR-4798-3p (SEQ ID NO: 1833), hsa-miR-4789-5p (SEQ ID NO: 1834), hsa-miR-4686 (SEQ ID NO: 1835), hsa-miR-133b (SEQ ID NO: 1836), hsa-miR-12128 (SEQ ID NO: 1837), hsa-miR-548ah-3p (SEQ ID NO: 1838), hsa-miR-3663-5p (SEQ ID NO: 1839), hsa-miR-4728-3p (SEQ ID NO: 1840), hsa-miR-410-5p (SEQ ID NO: 1841), hsa-miR-4664-3p (SEQ ID NO: 1842), hsa-miR-548q (SEQ ID NO: 1843), hsa-miR-15b-5p (SEQ ID NO: 1844), hsa-miR-4659b-5p (SEQ ID NO: 1845), hsa-miR-572 (SEQ ID NO: 1846), hsa-miR-4485-3p (SEQ ID NO: 1847), hsa-miR-4478 (SEQ ID NO: 1848), hsa-miR-4307 (SEQ ID NO: 1849), hsa-miR-3648 (SEQ ID NO: 1850), hsa-miR-6081 (SEQ ID NO: 1851), hsa-miR-10399-5p (SEQ ID NO: 1852), hsa-miR-937-5p (SEQ ID NO: 1853), hsa-miR-181d-3p (SEQ ID NO: 1854), hsa-miR-128-3p (SEQ ID NO: 1855), hsa-miR-6829-5p (SEQ ID NO: 1856), hsa-miR-7856-5p (SEQ ID NO: 1857), hsa-miR-4756-5p (SEQ ID NO: 1858), hsa-miR-3175 (SEQ ID NO: 1859), hsa-miR-3158-5p (SEQ ID NO: 1860), hsa-miR-4632-3p (SEQ ID NO: 1861), hsa-miR-1237-3p (SEQ ID NO: 1862), hsa-miR-4517 (SEQ ID NO: 1863), hsa-miR-155-3p (SEQ ID NO: 1864), hsa-miR-3128 (SEQ ID NO: 1865), hsa-miR-1268a (SEQ ID NO: 1866), hsa-miR-4661-3p (SEQ ID NO: 1867), hsa-miR-7152-3p (SEQ ID NO: 1868), hsa-miR-6828-3p (SEQ ID NO: 1869), hsa-miR-4444 (SEQ ID NO: 1870), hsa-miR-8056 (SEQ ID NO: 1871), hsa-miR-6757-5p (SEQ ID NO: 1872), hsa-miR-501-5p (SEQ ID NO: 1873), hsa-miR-6838-5p (SEQ ID NO: 1874), hsa-miR-342-5p (SEQ ID NO: 1875), hsa-miR-6758-5p (SEQ ID NO: 1876), hsa-miR-1229-3p (SEQ ID NO: 1877), hsa-miR-1284 (SEQ ID NO: 1878), hsa-miR-642a-3p (SEQ ID NO: 1879), hsa-miR-1229-5p (SEQ ID NO: 1880), hsa-miR-3185 (SEQ ID NO: 1881), hsa-miR-4270 (SEQ ID NO: 1882), hsa-miR-556-5p (SEQ ID NO: 1883), hsa-miR-4705 (SEQ ID NO: 1884), hsa-miR-519a-5p (SEQ ID NO: 1885), hsa-miR-5094 (SEQ ID NO: 1886), hsa-miR-759 (SEQ ID NO: 1887), hsa-miR-145-3p (SEQ ID NO: 1888), hsa-miR-375-5p (SEQ ID NO: 1889), hsa-miR-2116-5p (SEQ ID NO: 1890), hsa-miR-4741 (SEQ ID NO: 1891), hsa-miR-6789-5p (SEQ ID NO: 1892), hsa-miR-548t-5p (SEQ ID NO: 1893), hsa-miR-29c-3p (SEQ ID NO: 1894), hsa-let-7f-5p (SEQ ID NO: 1895), hsa-miR-483-5p (SEQ ID NO: 1896), hsa-miR-1539 (SEQ ID NO: 1897), hsa-miR-6842-5p (SEQ ID NO: 1898), hsa-miR-4745-5p (SEQ ID NO: 1899), hsa-miR-3675-3p (SEQ ID NO: 1900), hsa-miR-7111-5p (SEQ ID NO: 1901), hsa-let-7d-5p (SEQ ID NO: 1902), hsa-miR-10526-3p (SEQ ID NO: 1903), hsa-miR-12118 (SEQ ID NO: 1904), hsa-miR-30e-5p (SEQ ID NO: 1905), hsa-miR-1271-5p (SEQ ID NO: 1906), hsa-miR-555 (SEQ ID NO: 1907), hsa-miR-18a-3p (SEQ ID NO: 1908), hsa-miR-1255b-2-3p (SEQ ID NO: 1909), hsa-miR-378b (SEQ ID NO: 1910), hsa-miR-7161-5p (SEQ ID NO: 1911), hsa-miR-5007-3p (SEQ ID NO: 1912), hsa-miR-3915 (SEQ ID NO: 1913), hsa-miR-4462 (SEQ ID NO: 1914), hsa-miR-369-5p (SEQ ID NO: 1915), hsa-miR-6820-3p (SEQ ID NO: 1916), hsa-miR-5704 (SEQ ID NO: 1917), hsa-miR-4537 (SEQ ID NO: 1918), hsa-miR-4450 (SEQ ID NO: 1919), hsa-miR-4321 (SEQ ID NO: 1920), hsa-miR-4799-3p (SEQ ID NO: 1921), hsa-miR-4745-3p (SEQ ID NO: 1922), hsa-miR-4725-5p (SEQ ID NO: 1923), hsa-miR-6862-5p (SEQ ID NO: 1924), hsa-miR-654-5p (SEQ ID NO: 1925), hsa-miR-9851-5p (SEQ ID NO: 1926), hsa-miR-99a-3p (SEQ ID NO: 1927), hsa-miR-626 (SEQ ID NO: 1928), hsa-miR-10398-3p (SEQ ID NO: 1929), hsa-miR-130b-5p (SEQ ID NO: 1930), hsa-miR-487a-5p (SEQ ID NO: 1931), hsa-miR-6799-3p (SEQ ID NO: 1932), hsa-miR-3064-3p (SEQ ID NO: 1933), hsa-miR-6726-3p (SEQ ID NO: 1934), hsa-miR-548au-3p (SEQ ID NO: 1935), hsa-miR-939-5p (SEQ ID NO: 1936), hsa-miR-485-5p (SEQ ID NO: 1937), hsa-miR-4251 (SEQ ID NO: 1938), hsa-miR-9985 (SEQ ID NO: 1939), hsa-miR-7158-3p (SEQ ID NO: 1940), hsa-miR-4506 (SEQ ID NO: 1941), hsa-miR-6880-3p (SEQ ID NO: 1942), hsa-miR-144-5p (SEQ ID NO: 1943), hsa-miR-122-5p (SEQ ID NO: 1944), hsa-miR-548f-5p (SEQ ID NO: 1945), hsa-miR-206 (SEQ ID NO: 1946), hsa-miR-136-5p (SEQ ID NO: 1947), hsa-miR-4715-5p (SEQ ID NO: 1948), hsa-miR-6810-3p (SEQ ID NO: 1949), hsa-miR-8072 (SEQ ID NO: 1950), hsa-miR-4295 (SEQ ID NO: 1951), hsa-miR-5195-3p (SEQ ID NO: 1952), hsa-miR-12131 (SEQ ID NO: 1953), hsa-miR-3689f (SEQ ID NO: 1954), hsa-miR-1972 (SEQ ID NO: 1955), hsa-miR-340-5p (SEQ ID NO: 1956), hsa-miR-3120-5p (SEQ ID NO: 1957), hsa-miR-485-3p (SEQ ID NO: 1958), hsa-miR-632 (SEQ ID NO: 1959), hsa-miR-25-3p (SEQ ID NO: 1960), hsa-miR-4684-3p (SEQ ID NO: 1961), hsa-miR-6728-5p (SEQ ID NO: 1962), hsa-miR-147b-5p (SEQ ID NO: 1963), hsa-miR-4289 (SEQ ID NO: 1964), hsa-miR-3688-3p (SEQ ID NO: 1965), hsa-miR-6851-5p (SEQ ID NO: 1966), hsa-miR-455-3p (SEQ ID NO: 1967), hsa-miR-425-5p (SEQ ID NO: 1968), hsa-miR-181c-5p (SEQ ID NO: 1969), hsa-miR-6717-5p (SEQ ID NO: 1970), hsa-miR-5685 (SEQ ID NO: 1971), hsa-miR-3668 (SEQ ID NO: 1972), hsa-miR-338-3p (SEQ ID NO: 1973), hsa-miR-499b-5p (SEQ ID NO: 1974), hsa-miR-153-5p (SEQ ID NO: 1975), hsa-miR-4703-3p (SEQ ID NO: 1976), hsa-miR-211-5p (SEQ ID NO: 1977), hsa-miR-767-5p (SEQ ID NO: 1978), hsa-miR-101-2-5p (SEQ ID NO: 1979), hsa-miR-219a-5p (SEQ ID NO: 1980), hsa-miR-219a-1-3p (SEQ ID NO: 1981), hsa-miR-4436b-5p (SEQ ID NO: 1982), hsa-miR-5572 (SEQ ID NO: 1983), hsa-miR-3121-5p (SEQ ID NO: 1984), hsa-miR-4523 (SEQ ID NO: 1985), hsa-miR-140-3p (SEQ ID NO: 1986), hsa-miR-3659 (SEQ ID NO: 1987), hsa-miR-3679-3p (SEQ ID NO: 1988), hsa-miR-4526 (SEQ ID NO: 1989), hsa-miR-7850-5p (SEQ ID NO: 1990), hsa-miR-6134 (SEQ ID NO: 1991), hsa-miR-625-5p (SEQ ID NO: 1992), hsa-miR-369-3p (SEQ ID NO: 1993), hsa-miR-1287-3p (SEQ ID NO: 1994), hsa-miR-132-3p (SEQ ID NO: 1995), hsa-miR-1537-5p (SEQ ID NO: 1996), hsa-miR-6882-5p (SEQ ID NO: 1997), hsa-miR-5002-3p (SEQ ID NO: 1998), hsa-miR-639 (SEQ ID NO: 1999), hsa-miR-6756-5p (SEQ ID NO: 2000), hsa-miR-1908-3p (SEQ ID NO: 2001), hsa-miR-6811-3p (SEQ ID NO: 2002), hsa-miR-6514-3p (SEQ ID NO: 2003), hsa-miR-6738-3p (SEQ ID NO: 2004), hsa-miR-548 h-5p (SEQ ID NO: 2005), hsa-miR-595 (SEQ ID NO: 2006), hsa-miR-662 (SEQ ID NO: 2007), hsa-miR-130a-3p (SEQ ID NO: 2008), hsa-miR-4736 (SEQ ID NO: 2009), hsa-miR-5088-3p (SEQ ID NO: 2010), hsa-miR-5004-5p (SEQ ID NO: 2011), hsa-miR-142-3p (SEQ ID NO: 2012), hsa-miR-4474-5p (SEQ ID NO: 2013), hsa-miR-520d-5p (SEQ ID NO: 2014), hsa-miR-1205 (SEQ ID NO: 2015), hsa-miR-1537-3p (SEQ ID NO: 2016), hsa-miR-9898 (SEQ ID NO: 2017), hsa-miR-5187-5p (SEQ ID NO: 2018), hsa-miR-4428 (SEQ ID NO: 2019), hsa-miR-4646-3p (SEQ ID NO: 2020), hsa-miR-3529-5p (SEQ ID NO: 2021), hsa-miR-6809-3p (SEQ ID NO: 2022), hsa-miR-1-5p (SEQ ID NO: 2023), hsa-miR-27a-3p (SEQ ID NO: 2024), hsa-miR-671-5p (SEQ ID NO: 2025), hsa-miR-5007-5p (SEQ ID NO: 2026), hsa-miR-1285-5p (SEQ ID NO: 2027), hsa-miR-3919 (SEQ ID NO: 2028), hsa-miR-8059 (SEQ ID NO: 2029), hsa-miR-539-5p (SEQ ID NO: 2030), hsa-miR-3124-3p (SEQ ID NO: 2031), hsa-miR-33b-5p (SEQ ID NO: 2032), hsa-miR-7108-3p (SEQ ID NO: 2033), hsa-miR-3916 (SEQ ID NO: 2034), hsa-miR-4275 (SEQ ID NO: 2035), hsa-miR-6744-3p (SEQ ID NO: 2036), hsa-miR-3649 (SEQ ID NO: 2037), hsa-miR-3170 (SEQ ID NO: 2038), hsa-miR-6756-3p (SEQ ID NO: 2039), hsa-miR-4754 (SEQ ID NO: 2040), hsa-miR-5586-3p (SEQ ID NO: 2041), hsa-miR-490-5p (SEQ ID NO: 2042), hsa-miR-34c-5p (SEQ ID NO: 2043), hsa-miR-522-3p (SEQ ID NO: 2044), hsa-miR-4306 (SEQ ID NO: 2045), hsa-miR-3689b-5p (SEQ ID NO: 2046), hsa-miR-4515 (SEQ ID NO: 2047), hsa-miR-373-3p (SEQ ID NO: 2048), hsa-miR-365b-5p (SEQ ID NO: 2049), hsa-miR-4802-3p (SEQ ID NO: 2050), hsa-miR-548aj-3p (SEQ ID NO: 2051), hsa-miR-185-5p (SEQ ID NO: 2052), hsa-miR-6876-3p (SEQ ID NO: 2053), hsa-miR-3127-5p (SEQ ID NO: 2054), hsa-miR-6503-3p (SEQ ID NO: 2055), hsa-miR-3180-5p (SEQ ID NO: 2056), hsa-miR-5698 (SEQ ID NO: 2057), hsa-miR-4477b (SEQ ID NO: 2058), hsa-miR-548ah-5p (SEQ ID NO: 2059), hsa-miR-888-5p (SEQ ID NO: 2060), hsa-miR-7-2-3p (SEQ ID NO: 2061), hsa-miR-4520-2-3p (SEQ ID NO: 2062), hsa-miR-3123 (SEQ ID NO: 2063), hsa-miR-4693-5p (SEQ ID NO: 2064), hsa-miR-6077 (SEQ ID NO: 2065), hsa-miR-6807-3p (SEQ ID NO: 2066), hsa-miR-4283 (SEQ ID NO: 2067), hsa-miR-549a-3p (SEQ ID NO: 2068), hsa-miR-12121 (SEQ ID NO: 2069), hsa-miR-922 (SEQ ID NO: 2070), hsa-miR-4789-3p (SEQ ID NO: 2071), hsa-miR-6890-5p (SEQ ID NO: 2072), hsa-miR-4284 (SEQ ID NO: 2073), hsa-miR-7702 (SEQ ID NO: 2074), hsa-miR-513a-5p (SEQ ID NO: 2075), hsa-miR-6787-5p (SEQ ID NO: 2076), hsa-miR-3667-5p (SEQ ID NO: 2077), hsa-miR-450a-5p (SEQ ID NO: 2078), hsa-miR-3118 (SEQ ID NO: 2079), hsa-miR-6076 (SEQ ID NO: 2080), hsa-miR-1290 (SEQ ID NO: 2081), hsa-miR-3163 (SEQ ID NO: 2082), hsa-miR-7154-5p (SEQ ID NO: 2083), hsa-miR-663a (SEQ ID NO: 2084), hsa-miR-6824-5p (SEQ ID NO: 2085), hsa-miR-9-5p (SEQ ID NO: 2086), hsa-miR-548f-3p (SEQ ID NO: 2087), hsa-miR-378c (SEQ ID NO: 2088), hsa-miR-216a-3p (SEQ ID NO: 2089), hsa-miR-892c-3p (SEQ ID NO: 2090), hsa-miR-6831-5p (SEQ ID NO: 2091), hsa-miR-125a-5p (SEQ ID NO: 2092), hsa-miR-378e (SEQ ID NO: 2093), hsa-miR-3617-5p (SEQ ID NO: 2094), hsa-miR-3662 (SEQ ID NO: 2095), hsa-miR-604 (SEQ ID NO: 2096), hsa-miR-4278 (SEQ ID NO: 2097), hsa-miR-3140-3p (SEQ ID NO: 2098), hsa-miR-181b-2-3p (SEQ ID NO: 2099), hsa-miR-4540 (SEQ ID NO: 2100), hsa-miR-6130 (SEQ ID NO: 2101), hsa-miR-3907 (SEQ ID NO: 2102), hsa-miR-493-3p (SEQ ID NO: 2103), hsa-miR-5009-3p (SEQ ID NO: 2104), hsa-miR-4456 (SEQ ID NO: 2105), hsa-miR-99a-5p (SEQ ID NO: 2106), hsa-miR-4743-5p (SEQ ID NO: 2107), hsa-miR-196a-5p (SEQ ID NO: 2108), hsa-miR-4723-3p (SEQ ID NO: 2109), hsa-miR-6763-5p (SEQ ID NO: 2110), hsa-miR-6809-5p (SEQ ID NO: 2111), hsa-miR-890 (SEQ ID NO: 2112), hsa-miR-4667-3p (SEQ ID NO: 2113), hsa-miR-1247-5p (SEQ ID NO: 2114), hsa-miR-6512-5p (SEQ ID NO: 2115), hsa-miR-3613-3p (SEQ ID NO: 2116), hsa-miR-548a-5p (SEQ ID NO: 2117), hsa-miR-526b-3p (SEQ ID NO: 2118), hsa-miR-1180-5p (SEQ ID NO: 2119), hsa-miR-4669 (SEQ ID NO: 2120), hsa-miR-497-5p (SEQ ID NO: 2121), hsa-miR-4426 (SEQ ID NO: 2122), hsa-miR-3976 (SEQ ID NO: 2123), hsa-miR-3196 (SEQ ID NO: 2124), hsa-miR-6840-5p (SEQ ID NO: 2125), hsa-miR-6889-5p (SEQ ID NO: 2126), hsa-miR-642b-3p (SEQ ID NO: 2127), hsa-miR-363-3p (SEQ ID NO: 2128), hsa-miR-3620-3p (SEQ ID NO: 2129), hsa-miR-4265 (SEQ ID NO: 2130), hsa-miR-3154 (SEQ ID NO: 2131), hsa-miR-6869-5p (SEQ ID NO: 2132), hsa-miR-320a-5p (SEQ ID NO: 2133), hsa-miR-4685-5p (SEQ ID NO: 2134), hsa-miR-4520-3p (SEQ ID NO: 2135), hsa-miR-6853-3p (SEQ ID NO: 2136), hsa-miR-3978 (SEQ ID NO: 2137), hsa-miR-5683 (SEQ ID NO: 2138), hsa-miR-493-5p (SEQ ID NO: 2139), hsa-miR-4675 (SEQ ID NO: 2140), hsa-miR-629-5p (SEQ ID NO: 2141), hsa-miR-6776-3p (SEQ ID NO: 2142), hsa-miR-4423-5p (SEQ ID NO: 2143), hsa-miR-5009-5p (SEQ ID NO: 2144), hsa-miR-6780b-3p (SEQ ID NO: 2145), hsa-miR-655-3p (SEQ ID NO: 2146), hsa-miR-181d-5p (SEQ ID NO: 2147), hsa-miR-4443 (SEQ ID NO: 2148), hsa-miR-4528 (SEQ ID NO: 2149), hsa-miR-2116-3p (SEQ ID NO: 2150), hsa-miR-12117 (SEQ ID NO: 2151), hsa-miR-6895-5p (SEQ ID NO: 2152), hsa-miR-22-5p (SEQ ID NO: 2153), hsa-miR-3190-3p (SEQ ID NO: 2154), hsa-miR-548m (SEQ ID NO: 2155), hsa-miR-18b-3p (SEQ ID NO: 2156), hsa-miR-28-5p (SEQ ID NO: 2157), hsa-miR-6070 (SEQ ID NO: 2158), hsa-miR-323a-3p (SEQ ID NO: 2159), hsa-miR-8068 (SEQ ID NO: 2160), hsa-miR-3173-3p (SEQ ID NO: 2161), hsa-miR-6767-5p (SEQ ID NO: 2162), hsa-miR-6861-5p (SEQ ID NO: 2163), hsa-miR-8061 (SEQ ID NO: 2164), hsa-miR-519c-3p (SEQ ID NO: 2165), hsa-miR-6785-3p (SEQ ID NO: 2166), hsa-miR-129-5p (SEQ ID NO: 2167), hsa-miR-1295b-3p (SEQ ID NO: 2168), hsa-miR-4733-5p (SEQ ID NO: 2169), hsa-miR-9903 (SEQ ID NO: 2170), hsa-miR-3136-3p (SEQ ID NO: 2171), hsa-miR-7159-3p (SEQ ID NO: 2172), hsa-miR-658 (SEQ ID NO: 2173), hsa-miR-182-5p (SEQ ID NO: 2174), hsa-miR-5588-5p (SEQ ID NO: 2175), hsa-miR-548ak (SEQ ID NO: 2176), hsa-miR-3143 (SEQ ID NO: 2177), hsa-miR-6529-3p (SEQ ID NO: 2178), hsa-miR-504-5p (SEQ ID NO: 2179), hsa-miR-411-3p (SEQ ID NO: 2180), hsa-miR-4439 (SEQ ID NO: 2181), hsa-miR-7111-3p (SEQ ID NO: 2182), hsa-miR-514a-3p (SEQ ID NO: 2183), hsa-miR-6727-5p (SEQ ID NO: 2184), hsa-miR-4672 (SEQ ID NO: 2185), hsa-miR-4714-3p (SEQ ID NO: 2186), hsa-miR-4753-3p (SEQ ID NO: 2187), hsa-miR-1825 (SEQ ID NO: 2188), hsa-miR-3925-5p (SEQ ID NO: 2189), hsa-miR-132-5p (SEQ ID NO: 2190), hsa-miR-6509-5p (SEQ ID NO: 2191), hsa-miR-4699-5p (SEQ ID NO: 2192), hsa-miR-5787 (SEQ ID NO: 2193), hsa-miR-1909-3p (SEQ ID NO: 2194), hsa-miR-147b-3p (SEQ ID NO: 2195), hsa-miR-30c-5p (SEQ ID NO: 2196), hsa-miR-455-5p (SEQ ID NO: 2197), hsa-miR-4761-3p (SEQ ID NO: 2198), hsa-miR-5585-3p (SEQ ID NO: 2199), hsa-miR-4760-3p (SEQ ID NO: 2200), hsa-miR-1911-3p (SEQ ID NO: 2201), hsa-miR-6889-3p (SEQ ID NO: 2202), hsa-miR-6893-3p (SEQ ID NO: 2203), hsa-miR-195-3p (SEQ ID NO: 2204), hsa-miR-1303 (SEQ ID NO: 2205), hsa-miR-5589-3p (SEQ ID NO: 2206), hsa-miR-10392-5p (SEQ ID NO: 2207), hsa-miR-4649-3p (SEQ ID NO: 2208), hsa-miR-548aj-5p (SEQ ID NO: 2209), hsa-miR-4747-3p (SEQ ID NO: 2210), hsa-miR-7161-3p (SEQ ID NO: 2211), hsa-miR-1295a (SEQ ID NO: 2212), hsa-miR-376b-5p (SEQ ID NO: 2213), hsa-miR-4637 (SEQ ID NO: 2214), hsa-miR-6800-5p (SEQ ID NO: 2215), hsa-miR-506-5p (SEQ ID NO: 2216), hsa-miR-1182 (SEQ ID NO: 2217), hsa-miR-653-3p (SEQ ID NO: 2218), hsa-miR-6871-3p (SEQ ID NO: 2219), hsa-miR-299-5p (SEQ ID NO: 2220), hsa-miR-548p (SEQ ID NO: 2221), hsa-miR-6803-5p (SEQ ID NO: 2222), hsa-miR-4470 (SEQ ID NO: 2223), hsa-miR-6788-5p (SEQ ID NO: 2224), hsa-miR-1203 (SEQ ID NO: 2225), hsa-miR-3137 (SEQ ID NO: 2226), hsa-miR-615-3p (SEQ ID NO: 2227), hsa-miR-5691 (SEQ ID NO: 2228), hsa-miR-1185-2-3p (SEQ ID NO: 2229), hsa-miR-548ad-3p (SEQ ID NO: 2230), hsa-miR-6776-5p (SEQ ID NO: 2231), hsa-miR-524-5p (SEQ ID NO: 2232), hsa-miR-3685 (SEQ ID NO: 2233), hsa-miR-7977 (SEQ ID NO: 2234), hsa-miR-4787-5p (SEQ ID NO: 2235), hsa-miR-519d-3p (SEQ ID NO: 2236), hsa-miR-449c-3p (SEQ ID NO: 2237), hsa-miR-5581-3p (SEQ ID NO: 2238), hsa-miR-10b-3p (SEQ ID NO: 2239), hsa-miR-454-5p (SEQ ID NO: 2240), hsa-miR-548ay-3p (SEQ ID NO: 2241), hsa-miR-10400-5p (SEQ ID NO: 2242), hsa-miR-5579-3p (SEQ ID NO: 2243), hsa-miR-4451 (SEQ ID NO: 2244), hsa-miR-5100 (SEQ ID NO: 2245), hsa-miR-92a-3p (SEQ ID NO: 2246), hsa-miR-337-3p (SEQ ID NO: 2247), hsa-miR-6852-5p (SEQ ID NO: 2248), hsa-miR-4763-3p (SEQ ID NO: 2249), hsa-miR-302f (SEQ ID NO: 2250), hsa-miR-548i (SEQ ID NO: 2251), hsa-miR-660-5p (SEQ ID NO: 2252), hsa-miR-6089 (SEQ ID NO: 2253), hsa-miR-520a-5p (SEQ ID NO: 2254), hsa-miR-4533 (SEQ ID NO: 2255), hsa-miR-12136 (SEQ ID NO: 2256), hsa-miR-7153-5p (SEQ ID NO: 2257), hsa-miR-3155b (SEQ ID NO: 2258), hsa-miR-151a-3p (SEQ ID NO: 2259), hsa-miR-3941 (SEQ ID NO: 2260), hsa-miR-135a-2-3p (SEQ ID NO: 2261), hsa-miR-200c-5p (SEQ ID NO: 2262), hsa-miR-1178-5p (SEQ ID NO: 2263), hsa-miR-26b-3p (SEQ ID NO: 2264), hsa-miR-1468-5p (SEQ ID NO: 2265), hsa-miR-8077 (SEQ ID NO: 2266), hsa-miR-523-5p (SEQ ID NO: 2267), hsa-miR-6715b-5p (SEQ ID NO: 2268), hsa-miR-4796-3p (SEQ ID NO: 2269), hsa-miR-6794-3p (SEQ ID NO: 2270), hsa-miR-4262 (SEQ ID NO: 2271), hsa-miR-582-3p (SEQ ID NO: 2272), hsa-miR-4433a-3p (SEQ ID NO: 2273), hsa-miR-106b-3p (SEQ ID NO: 2274), hsa-miR-6854-3p (SEQ ID NO: 2275), hsa-miR-5571-5p (SEQ ID NO: 2276), hsa-miR-3148 (SEQ ID NO: 2277), hsa-miR-433-5p (SEQ ID NO: 2278), hsa-miR-4762-3p (SEQ ID NO: 2279), hsa-miR-1269b (SEQ ID NO: 2280), hsa-miR-942-5p (SEQ ID NO: 2281), hsa-miR-4324 (SEQ ID NO: 2282), hsa-miR-129-2-3p (SEQ ID NO: 2283), hsa-miR-6739-3p (SEQ ID NO: 2284), hsa-miR-3177-5p (SEQ ID NO: 2285), hsa-miR-4492 (SEQ ID NO: 2286), hsa-miR-4779 (SEQ ID NO: 2287), hsa-miR-3126-3p (SEQ ID NO: 2288), hsa-miR-6760-5p (SEQ ID NO: 2289), hsa-miR-5001-3p (SEQ ID NO: 2290), hsa-miR-3145-3p (SEQ ID NO: 2291), hsa-miR-7109-3p (SEQ ID NO: 2292), hsa-miR-4670-5p (SEQ ID NO: 2293), hsa-miR-98-5p (SEQ ID NO: 2294), hsa-miR-6827-5p (SEQ ID NO: 2295), hsa-miR-29a-3p (SEQ ID NO: 2296), hsa-miR-362-5p (SEQ ID NO: 2297), hsa-miR-7113-3p (SEQ ID NO: 2298), hsa-miR-1286 (SEQ ID NO: 2299), hsa-miR-549a-5p (SEQ ID NO: 2300), hsa-miR-505-3p (SEQ ID NO: 2301), hsa-miR-3675-5p (SEQ ID NO: 2302), hsa-miR-15a-5p (SEQ ID NO: 2303), hsa-miR-7855-5p (SEQ ID NO: 2304), hsa-miR-548as-5p (SEQ ID NO: 2305), hsa-miR-642b-5p (SEQ ID NO: 2306), hsa-miR-619-3p (SEQ ID NO: 2307), hsa-miR-550a-3-5p (SEQ ID NO: 2308), hsa-miR-3622b-5p (SEQ ID NO: 2309), hsa-miR-6777-5p (SEQ ID NO: 2310), hsa-miR-147a (SEQ ID NO: 2311), hsa-miR-302d-5p (SEQ ID NO: 2312), hsa-miR-10525-3p (SEQ ID NO: 2313), hsa-miR-432-5p (SEQ ID NO: 2314), hsa-miR-204-3p (SEQ ID NO: 2315), hsa-miR-3663-3p (SEQ ID NO: 2316), hsa-miR-5093 (SEQ ID NO: 2317), hsa-miR-1913 (SEQ ID NO: 2318), hsa-miR-4529-5p (SEQ ID NO: 2319), hsa-miR-4666b (SEQ ID NO: 2320), hsa-miR-6886-5p (SEQ ID NO: 2321), hsa-miR-4309 (SEQ ID NO: 2322), hsa-miR-3184-5p (SEQ ID NO: 2323), hsa-miR-4633-5p (SEQ ID NO: 2324), hsa-miR-4290 (SEQ ID NO: 2325), hsa-miR-345-3p (SEQ ID NO: 2326), hsa-miR-3176 (SEQ ID NO: 2327), hsa-miR-6824-3p (SEQ ID NO: 2328), hsa-miR-9986 (SEQ ID NO: 2329), hsa-miR-502-5p (SEQ ID NO: 2330), hsa-miR-1180-3p (SEQ ID NO: 2331), hsa-miR-4464 (SEQ ID NO: 2332), hsa-miR-5187-3p (SEQ ID NO: 2333), hsa-miR-3944-5p (SEQ ID NO: 2334), hsa-miR-664a-3p (SEQ ID NO: 2335), hsa-miR-548at-5p (SEQ ID NO: 2336), hsa-miR-591 (SEQ ID NO: 2337), hsa-miR-143-5p (SEQ ID NO: 2338), hsa-miR-4753-5p (SEQ ID NO: 2339), hsa-miR-383-5p (SEQ ID NO: 2340), hsa-miR-6885-5p (SEQ ID NO: 2341), hsa-miR-503-5p (SEQ ID NO: 2342), hsa-miR-6875-5p (SEQ ID NO: 2343), hsa-miR-1234-3p (SEQ ID NO: 2344), hsa-miR-19a-5p (SEQ ID NO: 2345), hsa-miR-6779-5p (SEQ ID NO: 2346), hsa-miR-1233-3p (SEQ ID NO: 2347), hsa-miR-6769b-5p (SEQ ID NO: 2348), hsa-miR-133a-5p (SEQ ID NO: 2349), hsa-miR-2861 (SEQ ID NO: 2350), hsa-miR-6501-3p (SEQ ID NO: 2351), hsa-miR-1260b (SEQ ID NO: 2352), hsa-miR-4536-3p (SEQ ID NO: 2353), hsa-miR-185-3p (SEQ ID NO: 2354), hsa-miR-4529-3p (SEQ ID NO: 2355), hsa-miR-1243 (SEQ ID NO: 2356), hsa-miR-4738-5p (SEQ ID NO: 2357), hsa-miR-761 (SEQ ID NO: 2358), hsa-miR-5190 (SEQ ID NO: 2359), hsa-miR-4712-3p (SEQ ID NO: 2360), hsa-miR-6835-5p (SEQ ID NO: 2361), hsa-miR-6750-3p (SEQ ID NO: 2362), hsa-miR-6778-3p (SEQ ID NO: 2363), hsa-miR-4481 (SEQ ID NO: 2364), hsa-miR-4636 (SEQ ID NO: 2365), hsa-miR-10393-5p (SEQ ID NO: 2366), hsa-miR-449b-5p (SEQ ID NO: 2367), hsa-miR-6719-3p (SEQ ID NO: 2368), hsa-miR-4652-5p (SEQ ID NO: 2369), hsa-miR-4318 (SEQ ID NO: 2370), hsa-miR-7153-3p (SEQ ID NO: 2371), hsa-miR-1262 (SEQ ID NO: 2372), hsa-miR-4795-3p (SEQ ID NO: 2373), hsa-miR-374b-5p (SEQ ID NO: 2374), hsa-miR-29c-5p (SEQ ID NO: 2375), hsa-miR-30c-1-3p (SEQ ID NO: 2376), hsa-miR-548w (SEQ ID NO: 2377), hsa-miR-616-3p (SEQ ID NO: 2378), hsa-miR-12127 (SEQ ID NO: 2379), hsa-miR-561-5p (SEQ ID NO: 2380), hsa-miR-4445-5p (SEQ ID NO: 2381), hsa-miR-4786-3p (SEQ ID NO: 2382), hsa-miR-23b-5p (SEQ ID NO: 2383), hsa-miR-802 (SEQ ID NO: 2384), hsa-miR-125b-5p (SEQ ID NO: 2385), hsa-miR-596 (SEQ ID NO: 2386), hsa-miR-3651 (SEQ ID NO: 2387), hsa-miR-6804-5p (SEQ ID NO: 2388), hsa-miR-545-5p (SEQ ID NO: 2389), hsa-miR-4773 (SEQ ID NO: 2390), hsa-miR-371b-5p (SEQ ID NO: 2391), hsa-miR-4468 (SEQ ID NO: 2392), hsa-miR-519d-5p (SEQ ID NO: 2393), hsa-miR-4653-5p (SEQ ID NO: 2394), hsa-miR-302c-3p (SEQ ID NO: 2395), hsa-miR-516b-5p (SEQ ID NO: 2396), hsa-miR-671-3p (SEQ ID NO: 2397), hsa-miR-1261 (SEQ ID NO: 2398), hsa-miR-545-3p (SEQ ID NO: 2399), hsa-miR-184 (SEQ ID NO: 2400), hsa-miR-7107-3p (SEQ ID NO: 2401), hsa-miR-93-3p (SEQ ID NO: 2402), hsa-miR-7151-3p (SEQ ID NO: 2403), hsa-miR-128-2-5p (SEQ ID NO: 2404), hsa-miR-135b-5p (SEQ ID NO: 2405), hsa-miR-195-5p (SEQ ID NO: 2406), hsa-miR-11181-5p (SEQ ID NO: 2407), hsa-miR-548j-3p (SEQ ID NO: 2408), hsa-miR-651-3p (SEQ ID NO: 2409), hsa-miR-125a-3p (SEQ ID NO: 2410), hsa-miR-328-5p (SEQ ID NO: 2411), hsa-miR-8057 (SEQ ID NO: 2412), hsa-miR-6798-5p (SEQ ID NO: 2413), hsa-let-7a-2-3p (SEQ ID NO: 2414), hsa-miR-4258 (SEQ ID NO: 2415), hsa-miR-224-3p (SEQ ID NO: 2416), hsa-miR-6819-3p (SEQ ID NO: 2417), hsa-miR-520d-3p (SEQ ID NO: 2418), hsa-miR-4447 (SEQ ID NO: 2419), hsa-miR-6752-5p (SEQ ID NO: 2420), hsa-miR-450b-5p (SEQ ID NO: 2421), hsa-miR-3182 (SEQ ID NO: 2422), hsa-miR-4704-5p (SEQ ID NO: 2423), hsa-miR-6894-5p (SEQ ID NO: 2424), hsa-miR-4271 (SEQ ID NO: 2425), hsa-miR-6747-5p (SEQ ID NO: 2426), hsa-miR-4683 (SEQ ID NO: 2427), hsa-miR-4790-3p (SEQ ID NO: 2428), hsa-miR-6737-5p (SEQ ID NO: 2429), hsa-miR-6082 (SEQ ID NO: 2430), hsa-miR-4691-3p (SEQ ID NO: 2431), hsa-miR-1228-5p (SEQ ID NO: 2432), hsa-miR-3654 (SEQ ID NO: 2433), hsa-miR-7114-3p (SEQ ID NO: 2434), hsa-miR-299-3p (SEQ ID NO: 2435), hsa-miR-636 (SEQ ID NO: 2436), hsa-miR-4254 (SEQ ID NO: 2437), hsa-miR-578 (SEQ ID NO: 2438), hsa-miR-3150b-5p (SEQ ID NO: 2439), hsa-miR-6834-5p (SEQ ID NO: 2440), hsa-miR-3174 (SEQ ID NO: 2441), hsa-miR-6739-5p (SEQ ID NO: 2442), hsa-miR-1206 (SEQ ID NO: 2443), hsa-miR-934 (SEQ ID NO: 2444), hsa-miR-548am-3p (SEQ ID NO: 2445), hsa-miR-6823-5p (SEQ ID NO: 2446), hsa-miR-3939 (SEQ ID NO: 2447), hsa-miR-424-5p (SEQ ID NO: 2448), hsa-miR-194-5p (SEQ ID NO: 2449), hsa-miR-6868-5p (SEQ ID NO: 2450), hsa-miR-6502-3p (SEQ ID NO: 2451), hsa-miR-3667-3p (SEQ ID NO: 2452), hsa-miR-1322 (SEQ ID NO: 2453), hsa-miR-6842-3p (SEQ ID NO: 2454), hsa-miR-5189-3p (SEQ ID NO: 2455), hsa-miR-4742-3p (SEQ ID NO: 2456), hsa-miR-4495 (SEQ ID NO: 2457), hsa-miR-190b-5p (SEQ ID NO: 2458), hsa-miR-548bb-5p (SEQ ID NO: 2459), hsa-miR-4518 (SEQ ID NO: 2460), hsa-miR-3609 (SEQ ID NO: 2461), hsa-miR-609 (SEQ ID NO: 2462), hsa-miR-3944-3p (SEQ ID NO: 2463), hsa-miR-548aw (SEQ ID NO: 2464), hsa-miR-7110-5p (SEQ ID NO: 2465), hsa-miR-548a-3p (SEQ ID NO: 2466), hsa-miR-7975 (SEQ ID NO: 2467), hsa-miR-6126 (SEQ ID NO: 2468), hsa-miR-4440 (SEQ ID NO: 2469), hsa-miR-1255a (SEQ ID NO: 2470), hsa-miR-378 h (SEQ ID NO: 2471), hsa-miR-7976 (SEQ ID NO: 2472), hsa-miR-3059-3p (SEQ ID NO: 2473), hsa-miR-1468-3p (SEQ ID NO: 2474), hsa-miR-4719 (SEQ ID NO: 2475), hsa-miR-4727-5p (SEQ ID NO: 2476), hsa-miR-620 (SEQ ID NO: 2477), hsa-miR-451b (SEQ ID NO: 2478), hsa-miR-6832-3p (SEQ ID NO: 2479), hsa-miR-4660 (SEQ ID NO: 2480), hsa-miR-619-5p (SEQ ID NO: 2481), hsa-miR-6838-3p (SEQ ID NO: 2482), hsa-miR-6127 (SEQ ID NO: 2483), hsa-miR-597-5p (SEQ ID NO: 2484), hsa-miR-550a-3p (SEQ ID NO: 2485), hsa-miR-3144-3p (SEQ ID NO: 2486), hsa-let-7e-5p (SEQ ID NO: 2487), hsa-miR-3127-3p (SEQ ID NO: 2488), hsa-miR-4510 (SEQ ID NO: 2489), hsa-miR-4708-5p (SEQ ID NO: 2490), hsa-miR-4301 (SEQ ID NO: 2491), hsa-miR-1227-5p (SEQ ID NO: 2492), hsa-miR-8088 (SEQ ID NO: 2493), hsa-miR-5189-5p (SEQ ID NO: 2494), hsa-let-7f-1-3p (SEQ ID NO: 2495), hsa-miR-583 (SEQ ID NO: 2496), hsa-miR-760 (SEQ ID NO: 2497), hsa-miR-8063 (SEQ ID NO: 2498), hsa-let-7c-3p (SEQ ID NO: 2499), hsa-miR-611 (SEQ ID NO: 2500), hsa-miR-203b-3p (SEQ ID NO: 2501), hsa-miR-4724-5p (SEQ ID NO: 2502), hsa-miR-1296-3p (SEQ ID NO: 2503), hsa-miR-4703-5p (SEQ ID NO: 2504), hsa-miR-4700-3p (SEQ ID NO: 2505), hsa-miR-4287 (SEQ ID NO: 2506), hsa-miR-6843-3p (SEQ ID NO: 2507), hsa-miR-3929 (SEQ ID NO: 2508), hsa-miR-631 (SEQ ID NO: 2509), hsa-miR-7846-3p (SEQ ID NO: 2510), hsa-miR-3921 (SEQ ID NO: 2511), hsa-miR-6737-3p (SEQ ID NO: 2512), hsa-miR-125b-1-3p (SEQ ID NO: 2513), hsa-miR-4774-5p (SEQ ID NO: 2514), hsa-miR-4435 (SEQ ID NO: 2515), hsa-miR-6798-3p (SEQ ID NO: 2516), hsa-miR-4266 (SEQ ID NO: 2517), hsa-miR-4769-3p (SEQ ID NO: 2518), hsa-miR-1910-5p (SEQ ID NO: 2519), hsa-miR-548ab (SEQ ID NO: 2520), hsa-miR-181a-3p (SEQ ID NO: 2521), hsa-miR-664a-5p (SEQ ID NO: 2522), hsa-miR-12132 (SEQ ID NO: 2523), hsa-miR-4279 (SEQ ID NO: 2524), hsa-miR-466 (SEQ ID NO: 2525), hsa-miR-9899 (SEQ ID NO: 2526), hsa-miR-219b-3p (SEQ ID NO: 2527), hsa-miR-4425 (SEQ ID NO: 2528), hsa-miR-16-2-3p (SEQ ID NO: 2529), hsa-miR-1225-5p (SEQ ID NO: 2530), hsa-miR-7843-3p (SEQ ID NO: 2531), hsa-miR-4288 (SEQ ID NO: 2532), hsa-miR-181a-5p (SEQ ID NO: 2533), hsa-miR-4662a-3p (SEQ ID NO: 2534), hsa-miR-4766-3p (SEQ ID NO: 2535), hsa-miR-1245b-3p (SEQ ID NO: 2536), hsa-miR-6749-3p (SEQ ID NO: 2537), hsa-miR-5695 (SEQ ID NO: 2538), hsa-miR-1827 (SEQ ID NO: 2539), hsa-miR-1299 (SEQ ID NO: 2540), hsa-miR-374c-3p (SEQ ID NO: 2541), hsa-miR-4701-3p (SEQ ID NO: 2542), hsa-miR-7162-3p (SEQ ID NO: 2543), hsa-miR-6131 (SEQ ID NO: 2544), hsa-miR-6845-3p (SEQ ID NO: 2545), hsa-miR-320b (SEQ ID NO: 2546), hsa-miR-505-5p (SEQ ID NO: 2547), hsa-miR-4729 (SEQ ID NO: 2548), hsa-miR-6781-5p (SEQ ID NO: 2549), hsa-miR-3085-5p (SEQ ID NO: 2550), hsa-miR-6822-5p (SEQ ID NO: 2551), hsa-miR-3664-5p (SEQ ID NO: 2552), hsa-miR-192-5p (SEQ ID NO: 2553), hsa-miR-548l (SEQ ID NO: 2554), hsa-miR-9902 (SEQ ID NO: 2555), hsa-miR-6830-3p (SEQ ID NO: 2556), hsa-miR-1283 (SEQ ID NO: 2557), hsa-miR-6729-3p (SEQ ID NO: 2558), hsa-miR-5006-3p (SEQ ID NO: 2559), hsa-miR-5689 (SEQ ID NO: 2560), hsa-miR-412-3p (SEQ ID NO: 2561), hsa-miR-130b-3p (SEQ ID NO: 2562), hsa-let-7c-5p (SEQ ID NO: 2563), hsa-miR-548z (SEQ ID NO: 2564), hsa-miR-203a-3p (SEQ ID NO: 2565), hsa-miR-377-3p (SEQ ID NO: 2566), hsa-miR-6783-3p (SEQ ID NO: 2567), hsa-miR-498-3p (SEQ ID NO: 2568), hsa-miR-4319 (SEQ ID NO: 2569), hsa-miR-6841-5p (SEQ ID NO: 2570), hsa-miR-374c-5p (SEQ ID NO: 2571), hsa-miR-199b-3p (SEQ ID NO: 2572), hsa-miR-6165 (SEQ ID NO: 2573), hsa-miR-454-3p (SEQ ID NO: 2574), hsa-miR-12120 (SEQ ID NO: 2575), hsa-miR-4639-3p (SEQ ID NO: 2576), hsa-miR-1278 (SEQ ID NO: 2577), hsa-miR-5699-3p (SEQ ID NO: 2578), hsa-miR-6865-5p (SEQ ID NO: 2579), hsa-miR-6746-3p (SEQ ID NO: 2580), hsa-miR-6720-3p (SEQ ID NO: 2581), hsa-miR-874-5p (SEQ ID NO: 2582), hsa-miR-6505-3p (SEQ ID NO: 2583), hsa-miR-6849-5p (SEQ ID NO: 2584), hsa-miR-4473 (SEQ ID NO: 2585), hsa-miR-1973 (SEQ ID NO: 2586), hsa-miR-6786-5p (SEQ ID NO: 2587), hsa-miR-3129-3p (SEQ ID NO: 2588), hsa-miR-888-3p (SEQ ID NO: 2589), hsa-miR-6866-5p (SEQ ID NO: 2590), hsa-miR-6888-5p (SEQ ID NO: 2591), hsa-miR-107 (SEQ ID NO: 2592), hsa-miR-5003-5p (SEQ ID NO: 2593), hsa-miR-4804-5p (SEQ ID NO: 2594), hsa-miR-300 (SEQ ID NO: 2595), hsa-miR-6507-5p (SEQ ID NO: 2596), hsa-miR-3678-5p (SEQ ID NO: 2597), hsa-miR-6873-3p (SEQ ID NO: 2598), hsa-miR-4733-3p (SEQ ID NO: 2599), hsa-miR-4775 (SEQ ID NO: 2600), hsa-miR-3156-3p (SEQ ID NO: 2601), hsa-miR-4671-3p (SEQ ID NO: 2602), hsa-miR-4658 (SEQ ID NO: 2603), hsa-miR-933 (SEQ ID NO: 2604), hsa-miR-7845-5p (SEQ ID NO: 2605), hsa-miR-518c-3p (SEQ ID NO: 2606), hsa-miR-4666a-3p (SEQ ID NO: 2607), hsa-miR-3940-3p (SEQ ID NO: 2608), hsa-miR-218-2-3p (SEQ ID NO: 2609), hsa-miR-513c-3p (SEQ ID NO: 2610), hsa-miR-6742-3p (SEQ ID NO: 2611), hsa-miR-519b-5p (SEQ ID NO: 2612), hsa-miR-212-5p (SEQ ID NO: 2613), hsa-miR-889-3p (SEQ ID NO: 2614), hsa-miR-2054 (SEQ ID NO: 2615), hsa-miR-6796-3p (SEQ ID NO: 2616), hsa-miR-6732-3p (SEQ ID NO: 2617), hsa-miR-15a-3p (SEQ ID NO: 2618), hsa-miR-6790-5p (SEQ ID NO: 2619), hsa-miR-3614-3p (SEQ ID NO: 2620), hsa-miR-574-5p (SEQ ID NO: 2621), hsa-miR-33a-3p (SEQ ID NO: 2622), hsa-miR-1909-5p (SEQ ID NO: 2623), hsa-miR-548g-3p (SEQ ID NO: 2624), hsa-miR-1226-5p (SEQ ID NO: 2625), hsa-miR-5580-3p (SEQ ID NO: 2626), hsa-miR-4657 (SEQ ID NO: 2627), hsa-miR-3610 (SEQ ID NO: 2628), hsa-miR-4767 (SEQ ID NO: 2629), hsa-miR-296-3p (SEQ ID NO: 2630), hsa-miR-655-5p (SEQ ID NO: 2631), hsa-miR-7160-5p (SEQ ID NO: 2632), hsa-miR-6720-5p (SEQ ID NO: 2633), hsa-miR-4423-3p (SEQ ID NO: 2634), hsa-miR-519c-5p (SEQ ID NO: 2635), hsa-miR-877-3p (SEQ ID NO: 2636), hsa-miR-6752-3p (SEQ ID NO: 2637), hsa-miR-5008-3p (SEQ ID NO: 2638), hsa-miR-27b-3p (SEQ ID NO: 2639), hsa-miR-208b-3p (SEQ ID NO: 2640), hsa-miR-487b-5p (SEQ ID NO: 2641), hsa-miR-649 (SEQ ID NO: 2642), hsa-miR-326 (SEQ ID NO: 2643), hsa-miR-8085 (SEQ ID NO: 2644), hsa-miR-24-2-5p (SEQ ID NO: 2645), hsa-miR-1185-1-3p (SEQ ID NO: 2646), hsa-miR-548g-5p (SEQ ID NO: 2647), hsa-miR-4800-5p (SEQ ID NO: 2648), hsa-miR-6086 (SEQ ID NO: 2649), hsa-miR-6846-3p (SEQ ID NO: 2650), hsa-miR-10226 (SEQ ID NO: 2651), hsa-miR-3529-3p (SEQ ID NO: 2652), hsa-miR-4661-5p (SEQ ID NO: 2653), hsa-miR-548as-3p (SEQ ID NO: 2654), hsa-miR-6817-5p (SEQ ID NO: 2655), hsa-miR-6500-5p (SEQ ID NO: 2656), hsa-miR-5591-3p (SEQ ID NO: 2657), hsa-miR-6754-3p (SEQ ID NO: 2658), hsa-miR-16-5p (SEQ ID NO: 2659), hsa-miR-3159 (SEQ ID NO: 2660), hsa-miR-1302 (SEQ ID NO: 2661), hsa-miR-217-5p (SEQ ID NO: 2662), hsa-miR-558 (SEQ ID NO: 2663), hsa-miR-514b-3p (SEQ ID NO: 2664), hsa-miR-4785 (SEQ ID NO: 2665), hsa-miR-4723-5p (SEQ ID NO: 2666), hsa-miR-3671 (SEQ ID NO: 2667), hsa-miR-7706 (SEQ ID NO: 2668), hsa-miR-4502 (SEQ ID NO: 2669), hsa-miR-4294 (SEQ ID NO: 2670), hsa-miR-512-5p (SEQ ID NO: 2671), hsa-miR-191-5p (SEQ ID NO: 2672), or hsa-miR-1976 (SEQ ID NO: 2673).

In certain embodiments, the first domain of the first strand comprises 5 or more contiguous nucleobases on the 3′ end of the hsa-miR as provided above wherein each U is optionally individually and independently at each occurrence T. In certain embodiments, the first domain of the first strand comprises the 6 or more contiguous nucleobases on the 3′ end of the hsa-miR. In certain embodiments, the first domain of the first strand comprises the 7 or more contiguous nucleobases on the 3′ end of the hsa-miR. In certain embodiments, the first domain of the first strand comprises the 8 or more contiguous nucleobases on the 3′ end of the hsa-miR. In certain embodiments, the first domain of the first strand comprises the 9 or more contiguous nucleobases on the 3′ end of the hsa-miR. In certain embodiments, the first domain of the first strand comprises the 10 or more contiguous nucleobases on the 3′ end of the hsa-miR. In certain embodiments, the first domain of the first strand comprises the 11 or more contiguous nucleobases on the 3′ end of the hsa-miR. In certain embodiments, the first domain of the first strand comprises the 12 or more contiguous nucleobases on the 3′ end of the hsa-miR. In certain embodiments, the first domain of the first strand comprises the 13 contiguous nucleobases on the 3′ end of the hsa-miR. In certain embodiments, the first domain of the first strand comprises the 14 or more contiguous nucleobases on the 3′ end of the hsa-miR. In certain embodiments, the first domain of the first strand comprises the 15 or more contiguous nucleobases on the 3′ end of the hsa-miR. In certain embodiments, the first domain of the first strand comprises the 16 or more contiguous nucleobases on the 3′ end of the hsa-miR.

In certain embodiments, the second domain of the second strand is less than 16, 15, 14, 13, 12, 11, 10, 9, or 8 nucleobases.

In certain embodiments, the third domain of the second strand is between 12 and 4 nucleobases. In certain embodiments, the third domain of the second strand is less than 12, 11, 10, 9, 8, or 7 nucleobases, and more than 4, 5, 6, or 7 nucleobases.

In certain embodiments, the third domain of the second strand comprises 4 or more contiguous nucleobases that complement 4, 5, 6, or 7 or more nucleobases on the 5′ end of the hsa-miR.

In certain embodiments, the target RNA is messenger RNA. In certain embodiments, the target RNA is HIF1alpha messenger RNA.

In certain embodiments, this disclosure contemplates a conditional double stranded antisense oligonucleotide disclosed herein comprising/conjugated to a label.

In certain embodiments, this disclosure contemplates a particle comprising a conditional double stranded antisense oligonucleotide disclosed herein. In certain embodiments, the particle is a liposomal, viral, saccharide/polysaccharide or protein particle encapsulating the conditional double stranded antisense oligonucleotide.

In certain embodiments, this disclosure contemplates a pharmaceutical composition comprising a conditional double stranded antisense oligonucleotide or particle comprising the same as disclosed herein and a pharmaceutically acceptable excipient.

In certain embodiments, this disclosure contemplates an aqueous solution comprising a conditional double stranded antisense oligonucleotide disclosed herein or particles thereof optionally comprising salts, buffing agents, and/or saccharides or polysaccharides. In certain embodiments, this disclosure contemplates a pharmaceutical composition in the form of a tablet, pill, capsule, gel, gel capsule, or powder.

For therapeutic uses, the conditional double stranded antisense oligonucleotide or particle comprising the same as described herein may be formulated with a pharmaceutically acceptable excipient, such as physiological saline. Routes of administration for the compositions or agents described herein include systemic, subcutaneous, intravenous, intraperitoneal, intramuscular, or intradermal injections that provide continuous and sustained levels of the drug in the human patient.

Generally, for pharmaceutical use, the conditional double stranded antisense oligonucleotide or particle comprising the same may be formulated as a pharmaceutical preparation comprising a conditional double stranded antisense oligonucleotide or particle comprising the same and at least one pharmaceutically acceptable carrier, diluent, excipient and/or adjuvant, and optionally one or more further pharmaceutically active compounds.

The pharmaceutical preparations of the disclosure are preferably in a unit dosage form, and may be suitably packaged, for example, in a box, blister, vial, bottle, sachet, ampoule, or any other suitable single-dose or multi-dose holder or container (which may be properly labeled); optionally with one or more leaflets containing product information and/or instructions for use. Depending on the condition to be prevented or treated and the route of administration, such an effective amount will usually be between 0.1 and 500 mg per kilogram body weight of the patient per day, which may be administered as a single dose, divided over one or more daily doses. The amount(s) to be administered, the route of administration, and any further treatment regimen may be determined by the treating clinician, depending on factors such as age, gender, and general condition of the patient and the nature and severity of the disease/symptoms to be treated. Reference is made to U.S. Pat. Nos. 6,372,778, 6,369,086, 6,369,087, 6,372,733, as well as to the standard handbooks, such as the latest edition of Remington's Pharmaceutical Sciences.

Depending upon the manner of administration, the conditional double stranded antisense oligonucleotide or particle comprising the same as described herein may be formulated in a variety of ways. Solid dosage forms for oral administration include, but are not limited to, tablets, soft or hard gelatin or non-gelatin capsules, and caplets. However, liquid dosage forms, such as solutions, syrups, suspensions, shakes, etc. can also be utilized.

The carrier is all components present in the pharmaceutical formulations other than the active ingredient or ingredients. As generally used herein, “carrier” includes, but is not limited to, diluents, binders, lubricants, disintegrators, fillers, pH-modifying agents, preservatives, antioxidants, solubility enhancers, and coating compositions.

The carrier also includes all components of the coating composition which may include plasticizers, pigments, colorants, stabilizing agents, and glidants. Delayed release, extended release, and/or pulsatile release dosage formulations may be prepared as described in standard references, such as “Pharmaceutical dosage form tablets”, eds. Liberman et al. (New York, Marcel Dekker, Inc., 1989), “Remington—The science and practice of pharmacy”, 20^{th} et., Lippincott Williams & Wilkins, Baltimore, MD, 2000, and “Pharmaceutical dosage forms and drug delivery provide information on carriers, materials, equipment, and process for preparing tablets and capsules and delayed release dosage forms of tablets, capsules, and granules.

In certain embodiments, this disclosure contemplates kits comprising a conditional double stranded antisense oligonucleotide or particle comprising the same as disclosed herein and optionally a container, and optionally a pharmaceutically acceptable excipient. In certain embodiments, the container is a vial, capsule, syringe, or bottle/vial adapted with a septum for drawing liquid contents with a needle, syringe, cannula, or other transfer device.

In certain embodiments, this disclosure relates to methods of treating or preventing a disease or condition associated with target nucleic acid/RNA overexpression in a cell characterized by cell specific nucleic acid/RNA expression comprising administering to a subject in need thereof an effective amount of a conditional double stranded nucleobase polymer complex as disclosed herein.

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor. In certain embodiments, the caner is a hematological cancer such as lymphoma, leukemia, or multiple myeloma. Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, and thorax.

In certain embodiments, the method of administration is in a subject with a lymphodepleted environment due to prior or concurrent administration of lymphodepleting agents. In certain embodiments, lymphodepleting agents (e.g., cyclophosphamide and fludarabine).

In certain embodiments, the disease or condition is a cancer, cardiovascular disease, liver disease, type I diabetes, or type II diabetes. Contemplated cardiovascular diseases include calcific aortic valve disease (CAVD), atherosclerosis, myocardial infarction, stroke, congestive heart failure, or arrhythmia.

Design of miRNA-Inducible ASO

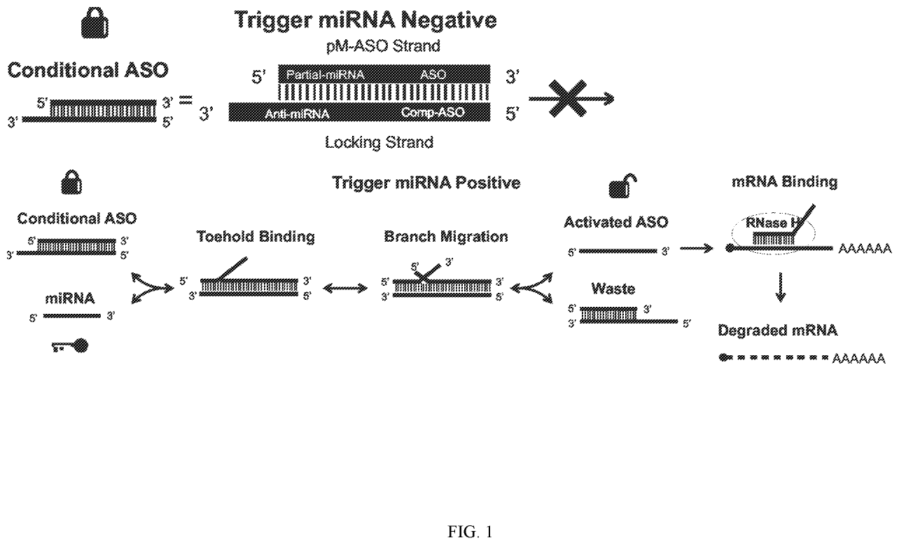

Conditional ASO are silenced due to the sequestration of the ASO sequence; however, upon triggering by the miRNA input, the conditional ASO was activated, allowing for binding and degradation of the target mRNA. The conditional ASO is a duplex formed by an extended ASO strand and a locking strand. The extended ASO strand was comprised of two domains: the parental ASO and a partial miRNA sequence at the 5′ terminus, i.e., the partial miRNA-ASO (pM-ASO) strand. The partial miRNA domain lacks the key seeding sequences (2-8 nt) of the miRNA, and hence avoids introducing the seeding sequence which may knockdown the miRNA target genes. The locking strand is composed of the complementary sequence to the entire miRNA and the parental ASO. Once hybridized, the two strands form a duplex with a single stranded toehold domain (FIG. 1). In the absence of the trigger miRNA, the duplex remains hybridized and the activity of the ASO is inhibited; whereas after internalization of the duplex by the trigger miRNA expressing cells, the miRNA binds to the toehold domain of the locking strand and thus initiates a competition reaction between miRNA and pM-ASO strand for the binding to the locking strand. If the binding of miRNA to the locking strand is favorable, the pM-ASO is displaced to expose and activate the ASO sequence. The activated ASO can then bind to and recruit RNase H to cleave the target mRNA (FIG. 1). Specifically, in the case of miR-122 inducible HIF1α ASO, the parental HIF1α ASO for conditional ASO is EZN2968, which is a 16 nt oligonucleotide with phosphorothioate (PS) backbone and locked nucleic acid (LNA) modification. miR-122-inducible HIF1α ASO is a duplex formed by the partial miR-122-EZN2968 (pM-EZN) strand and the locking strand.

### Optimization of Duplex Conformation of Conditional EZN2968

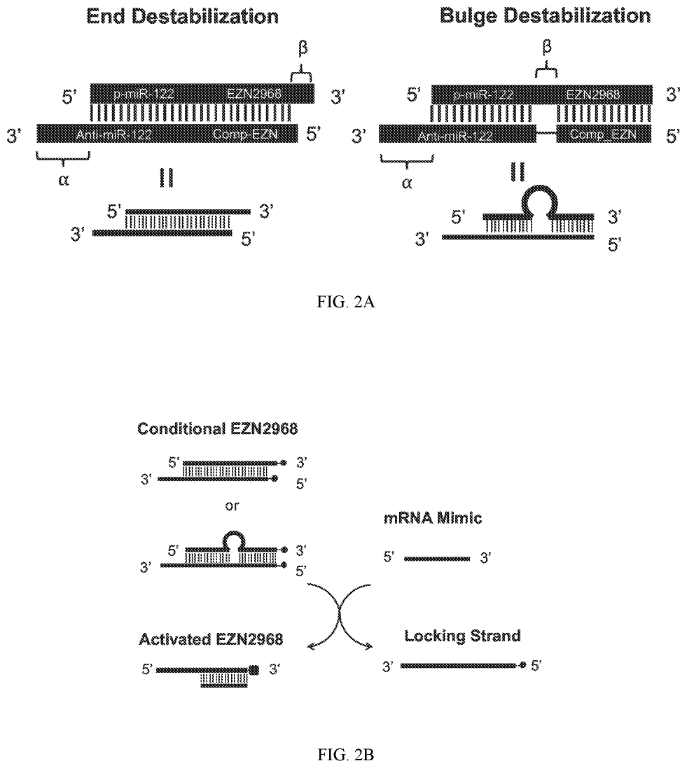

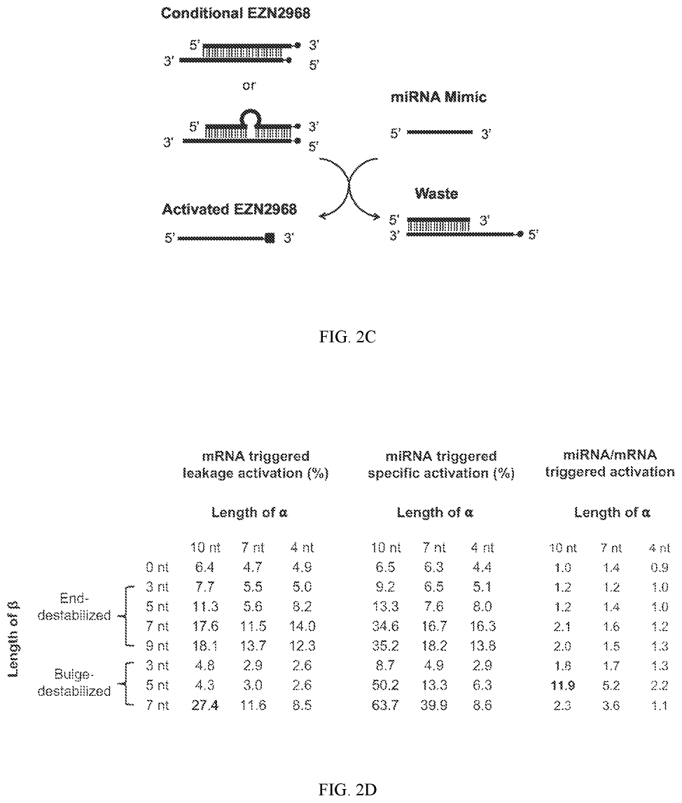

To create a selective and efficient miR-122 inducible EZN2968 ASO, the duplex was optimized for 1) minimum spontaneous dissociation (leakage) of the duplex in the absence of miR-122 to keep the HIF1α ASO activity low, and 2) a high miR-122 sensitivity that leads to maximum activation of the HIF1α ASO. These criteria can be met when the free energy (ΔG) of the conditional EZN2968 duplex is lower than the ΔG of pM-EZN/HIF1α mRNA duplex and higher than the ΔG of miR-122/locking strand duplex. The displacement mediated by the toehold (α domain) reduce the kinetic barrier for miR-122 triggered activation. Due to the thermostability of the completely locked duplex, its miR-122 sensitivity would be limited. To rationally enhance sensitivity to miR-122, several nucleotides can be removed (length of β domain) from the complementary sequence of EZN2968, rendering the duplex either in an end-destabilized conformation or in a bulge-destabilized conformation (FIG. 2A). The single stranded β domain may also function as a toehold and drive the separation of conditional EZN2968 upon binding of HIF1α mRNA reducing the kinetic barrier for HIF1α mRNA triggered leakage activation. Therefore, optimization of the duplex conformation and length of each domain is desired. Bulge destabilization is advantageous over end destabilization in terms of lower leakage activation triggered by HIF1α mRNA due to the relative inaccessibility of the bulge region for mRNA binding.

A library of chemically unmodified duplexes with different lengths (4 nt, 7 nt and 10 nt) of the toehold (α domain) and numbers of nucleotides removed in the duplex region (length of β domain) for both conformations. The duplexes were labeled with Cy5 on the 3′ termini of the pM-EZN strand and a quencher on the 5′ termini of the locking strand. Their de-hybridization upon triggering by a HIF1α mRNA mimicking strand was quantified by measuring the increase in fluorescence intensity driven by de-quenched Cy5 (FIG. 2B). The results showed that the percentage of displaced pM-EZN for all bulge destabilized duplexes were lower than their end destabilized counterparts, except for the ones with a 7 nt bulge and 7 nt or 10 nt toehold (FIG. 2D). The high leakage of 7 nt bulge duplexes may be attributed to lower thermodynamic stability because of the shorter double stranded domains as well as the lower stability of duplexes with larger bulge sizes. miRNA triggered activation was also measured with a similar assay, where the Cy5 and quencher-labeled duplexes were incubated with miR-122 mimicking sequence (FIG. 2C). The result showed that the miRNA triggered activation increased as the number of nucleotides removed from the duplex region (length of R domain) was increased for both conformations (FIG. 2D), due to the reduced stability of the duplexes indicated by calculated ΔG. Based on these results showing low HIF1α mRNA triggered leakage activation and high miR-122 sensitivity, or in other words, a higher miRNA to mRNA triggered activation ratio of the bulge-destabilized conformation. The bulge-destabilized duplex conformations were evaluated in vitro.

pM-EZN Strands Knock Down HIF1α in a Dose and Time Dependent Manner

To evaluate the potency of pM-EZN strands composed of partial miR-122 sequences for HIF1α knockdown as a benchmark, pM-EZN with 12 nt (pM12-EZN) or 15 nt (pM15-EZN) extension on the 5′ termini of EZN2968 were created. The pM-EZN strands were maintained the LNA modification and the PS backbone as the parental EZN2968. pM12-EZN or pM15-EZN were transfected in U373 cells, a glioblastoma cell line that expresses high level of HIF1α and negligible level of miR-122 and incubated for different time periods. These two strands knocked down HIF1α mRNA in a dose and time dependent manner, with a remarkable nearly complete knockdown at the condition of 200 nM concentration and 48 h incubation time. In addition, pM12-EZN and pM15-EZN knocked down HIF1α on both mRNA and protein level with similar potency. To confirm the inhibition of HIF1α activity by pM-EZN strands, LN229-V6R-Luc cells, which stably express luciferase under the control of a promoter containing Hypoxia Response Element (HRE) to report HIF1α activity, were transfected. Since this cell line expresses low amounts of luciferase at normoxic conditions, IOX4, a prolyl-hydroxylase 2 (PHD2) inhibitor, was added to inhibit HIF1α degradation and induce luciferase expression. Consistent with mRNA knockdown in U373 cells, transfection of 10 nM pM12-EZN and pM15-EZN led to significant reduction of luciferase expression in this reporter cell line.

Screening for Conditional EZN2968 with Minimum Spontaneous Leakage In Vitro

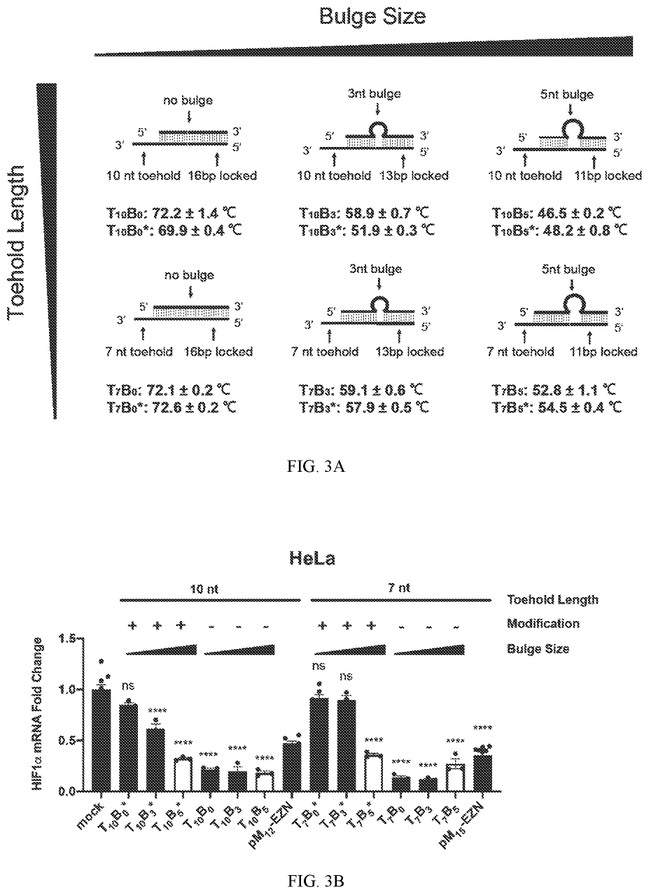

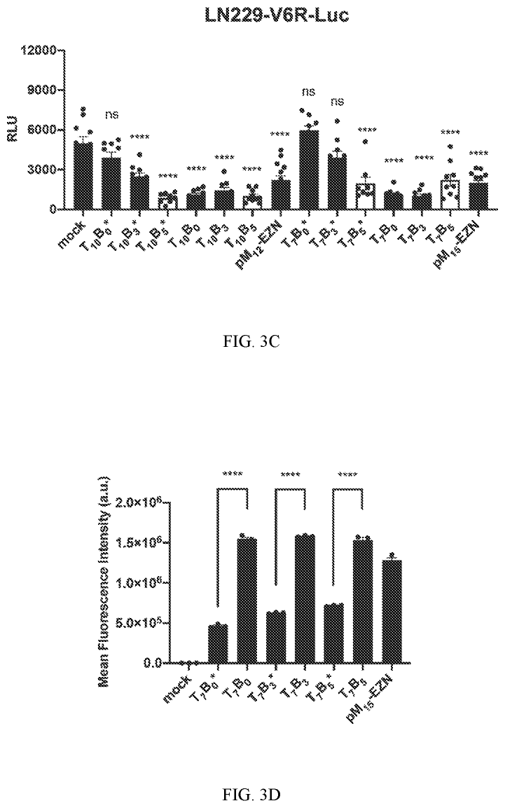

Experiments were performed to investigate the effect of chemical modification, bulge size and toehold length on the efficacy of locking strand in terms of spontaneous leakage of HIF1α knockdown activity. A library of 12 conditional EZN2968s were created by annealing pM12-EZN or pM15-EZN with 6 different locking strands (B0, B3, B5, B0*, B3*, and B5*) at a 1:1 ratio in PBS. Chemically modified (annotated with “*”) and unmodified locking strands were also compared to assess the role of nucleases in competition with the locking strand to inhibit EZN2968 activity. The locking strands are named based on the size of the bulge when hybridized to pM-EZN strand and the chemical modification. For example, B0 is the unmodified locking strand that does not generate a bulge when hybridized to pM-EZN strands; whereas B3* represents the chemically modified locking strand forming a 3 nt bulge duplex when hybridized to pM-EZN strands. Because backbone phosphorothioate (PS) modifications are reported to reduce affinity for complementary nucleic acid by about 0.5° C. per incorporation, LNA modifications were also incorporated in the locking strands to compensate for and maintain the thermodynamic stability of the duplex, i.e., it displays a similar melting temperature (Tm) compared to its unmodified counterpart. The resulting duplex library included permutations with a bulge size of 0 nt, 3 nt or 5 nt, a toehold length of 7 nt or 10 nt, and chemically modified (PS/LNA) or unmodified locking strands (FIG. 3A). The duplexes are termed based on the toehold length and bulge size of the duplex. For example, the duplex formed by pM12-EZN and B3* has a 10 nt toehold and a 3nt bulge, therefore it is named as T10B3*. The sequences are provided in the tables below.

TABLE 1
Oligonucleotide sequences for in buffer testing
of leakage activation of bulge and
end destabilized conditional EZN2968;
/3AmMO/ = 3′ amino modification;
/5IAbRQ/ = 5′ Iowa Black RQ;
/5AmMC6/ = 5′ amino modification;
“T” indicates a mismatch on the toehold
binding region.

| | Sequence | ID |
|---|---|---|
| Unmodified | CAA TGG TGT TTG TGG CAA | SEQ ID NO: 1 |
| pMI-EZN | GCA TCC IGT A/3AmMO/ | |
| Unmodified | TGA CAA TGG TGT TTG TGG | SEQ ID NO. 2 |
| pMI-EZN | CAA GCA TCC TGT A/3AmMO/ | |
| Unmodified | GTG TGA CAA TGG TGT TTG | SEQ ID NO: 3 |
| pMis-EZN | TGG CAA GCA TCC TGT A/3AmMO/ | |
| Unmodified B₀ | /5IAbRQ/TA CAG GAT GCT TGC CAC | SEQ ID NO: 4 |
| | AAA CAC CAT TGT CAC TCT CCA | |
| Unmodified B₃ | /5IAbRQ/TA CAG GAT GCT TGC AAA | SEQ ID NO: 5 |
| | CAC CAT TGT CAC TCT CCA | |
| Unmodified B₅ | /5IAbRQ/TA CAG GAT GCT CAA | SEQ ID NO: 6 |
| | ACA CCA TTG TCA CTC TCC A | |
| Unmodified B₇ | /5IAbRQ/TA CAG GAT GCA AAC | SEQ ID NO: 7 |
| | ACC ATT GTC ACT CTC CA | |
| Unmodified E₃ | /5IAbRQ/AG GAT GCT TGC CAC | SEQ ID NO: 8 |
| | AAA 5IAbRQCAT TGT CAC TCT CCA | |
| Unmodified E₅ | /5IAbRQ/GA TGC TTG CCA CAA | SEQ ID NO: 9 |
| | ACA CCA TTG TCA CTC TCC A | |
| Unmodified E₇ | /5IAbRQ/TG CTT GCC ACA AAC | SEQ ID NO: 10 |
| | ACC ATT GTC ACT CTC CA | |

Transfection experiments in HeLa cells showed that PS/LNA chemical modification of the locking strand was needed for maintaining the ASO in the inactive state. HeLa cells do not express miR-122. As a result, all the unmodified locking strands B0, B3, and B5 failed to inhibit ASO activity as measured by RT-qPCR and using the luciferase reporter cell line. Importantly, incorporating locking strands with the PS and LNA modifications (see table 2) showed substantial improvement, resulting in dampened knockdown of HIF1α as measured by RT-qPCR and the luciferase reporter (FIGS. 3B and 3C). Given that the Tm of B0 and B0* against the pM-EZN strands are similar, this indicates that the differential response is not driven by thermodynamic difference between the conventional nucleobases and the PS/LNA nucleic acids.

TABLE 2
Oligonucleotide sequences for in vitro testing of conditional EZN2968. “+” = LNA
modification; “*” = PS modification; /3AmMO/ = 3′ amino modification;
“T” indicates a mismatch on the toehold binding region.

| | Sequence | ID |
|---|---|---|
| EZN2968 | +T*+G*+G* C*A*A* G*C*A* T*C*C* +T*+G*+T* A | SEQ ID NO: 11 |
| EZN3088 | *C*+G*+T* C*A*G* T*A*T* G*C*G* +A*+A*+T* C | SEQ ID NO: 12 |
| Modified pM₁₂-EZN | C*A*A* T*G*G* T*G*T* T*T*G* +T*G*+G* C*A*A* G*C*A* | SEQ ID NO: 1 |
| | T*C*C* +T*+G*+T* A/3AmMO/ | |
| Modified pM/₁₅-EZN | T*G*A* C*A*A* T*G*G* T*G*T* T*T*G* +T*+G*+G* C*A*A* | SEQ ID NO: 2 |
| | G*C*A* T*C*C* +T*+G*+T* A/3AmMO/ | |
| Modified B₆ (B₆*) | /5IAbRQ/+T*+A* +C*A*G* G*A*T* G*C*T* T*G*C* C*A*C* | SEQ ID NO: 13 |
| | A*A*A* C*A*C* C*A*T* T*G*+T* +C*+A*C* A*C*T* C*C*A | |
| Modified B₃ (B₃*) | /5IAbRQ/*T*A* C*A*G* G*A*T* G*C*++T* +T*+G*+C* | SEQ ID NO. 14 |
| | +A*A*A* C*A*C* C*A*T* T*G*T* C*A*C* A*C*T* C*C*A | |
| Modified B₅ (B₅*) | /5IAbRQ/*T*A* C*A*G* G*A*T* +G*+C*+T* +C*+A*+A* | SEQ ID NO: 15 |
| | A*C*A*C*C*A* T*T*G* T*C*A* C*T*C* T*C*C* A | |
| B₃* without | /5IAbRQ/T*A* C*A*G* G*A*T* G*C*+T* +T*+G*+C* | SEQ ID NO: 16 |
| toehold | +A ** A*A* C*A*C* C*A*T* T*G*T* C*A | |
| miR-122 mimic | +T*G*G* +A*G*T* +G*T*G* +A*C*A* +A*T*G* +G*I*G* | SEQ ID NO: 17 |
| | +T*T*T* +G | |
| Scrambled miR-122 | +G*A*A*+G*T*A*+T*G*T*+G*G*T*+G*A*T*+T*G*C*+G*T* | SEQ ID NO: 18 |
| | G*+T | |
| Scr. 1-7nt | +G*G*G*+T*T*G*+A*T*G*+A*C*A*+A*T*G*+G*T*G*+T*T* | SEQ ID NO: 19 |
| miR-122 | T*+G | |

The failure in locking efficacy by unmodified locking strands may be due to their nuclease susceptibility, which leads to degradation of the locking strands and the spontaneous activation of pM-EZN strand. To test this, HeLa cells were transfected with pM15-EZN duplexes labeled with Cy5 and quencher pair and measured the cell-associated fluorescence by flow cytometry. Cells transfected with pM15-EZN duplexes with unmodified locking strands (T7B0, T7B3, T7B5) resulted in similar and even slightly higher levels of fluorescence intensity compared to single-stranded pM15-EZN alone transfected cells, indicating complete dissociation of the locking strand and pM15-EZN strand. The slightly higher level of fluorescence intensity was consistent with the higher HIF1α knockdown efficacy by the duplexes with unmodified locks compared to pM15-EZN only group. This may be due to higher transfection efficiency of double stranded DNA compared to single stranded DNA in HeLa cells. In contrast, pM15-EZN duplexes with modified locking strands (T7B0*, T7B3*, T7B5*) resulted in reduced fluorescence intensity compared to the pM15-EZN only group, showing that the duplexes remain primarily locked (hybridized) 24 h after transfection (FIG. 3D).

The experiments conducted using HeLa (lacking miR-122) also showed that increasing bulge size decreases the thermodynamic stability of the duplex as measured by Tm (FIG. 3A) and increased the level of spontaneous activation of the conditional ASO. T7B3* did not knockdown HIF1α, whereas T10B3* knocked down HIF1α significantly, indicating that the length of toehold and branch migration domain also plays an important role in the spontaneous leakage.

The conclusions that chemical modification of the locking strand and stable binding to the ASO (Tm>58° C.) are important to inhibit EZN2968 were further validated in U373 cells, lacking miR-122 expression, by both RT-qPCR and Western Blot analysis. T10B0*, T7B0* and T7B3* duplexes were used for testing miR-122 induced HIF1α knockdown because of low spontaneous activation.

Synthetic miR-122 Mimic Triggers Activation of Conditional EZN2968 In Vitro

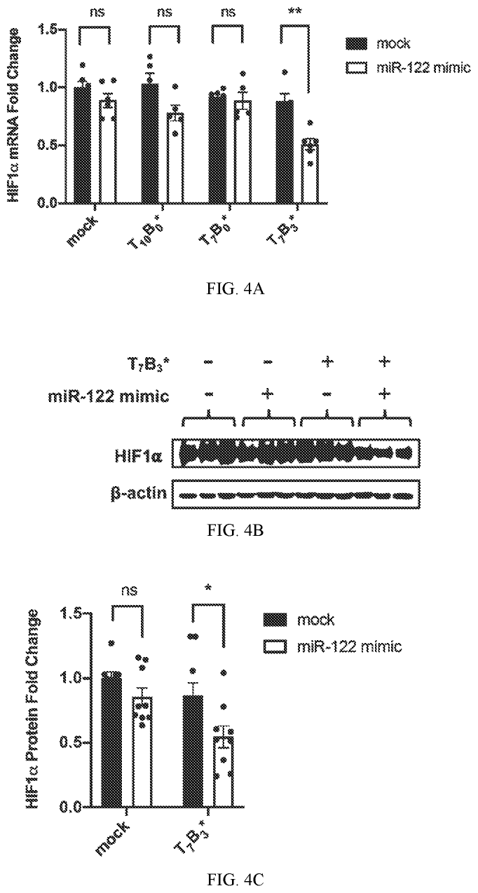

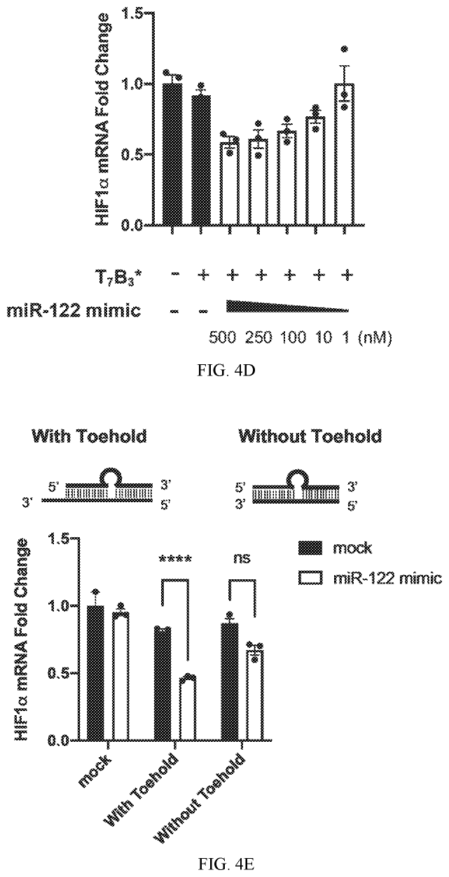

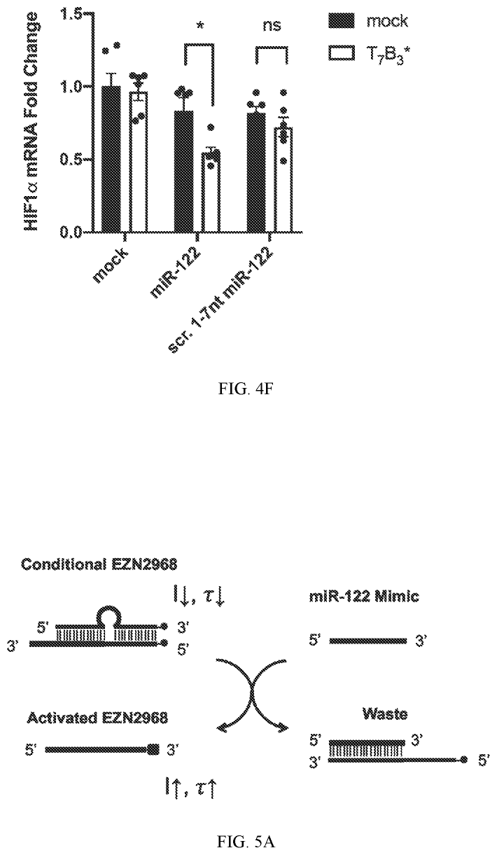

Experiments were performed to determine whether conditional EZN2968 could be activated in vitro by using an exogenously transfected miR-122 mimicking oligonucleotide (miR-122 mimic, table 2). For this purpose, the cells were co-transfected with the conditional EZN2968 along with the miR-122 mimic in U373 cells. To minimize the complexity of the experiment, the transfection mixture of the conditional EZN2968 duplex (T7B0* or T7B3*) and miR-122 mimic separately was prepared with Oligofectamine™. The two solutions were added to cells simultaneously. After 24 h incubation, 10 nM T7B3* alone did not show significant down regulation of HIF1α mRNA, whereas 10 nM T7B3* co-transfected with 500 nM miR-122 mimic down regulated HIF1α mRNA by ˜50% (FIG. 4A). In contrast, T7B0*, which has no bulge and higher thermodynamic stability, did not knockdown HIF1α mRNA in these conditions. These results indicate completely locked duplexes without a bulge render low miR-122 sensitivity, and the destabilization of the duplex is necessary for enhancing miR-122 sensitivity. Co-transfection of T7B3* and miR-122 mimic down regulated HIF1α protein levels in U373 cells after 24 h incubation (FIGS. 4B and 4C). It should be noted that the conclusions are time dependent, and when the incubation time was extended to 48 h, T7B3* showed more spontaneous knockdown of HIF1α mRNA, and T7B0* showed significant miR-122-triggered activation.

Given the miR-122 sensitivity of T7B3*, its dose-dependent response to miR-122 was quantified. HIF1α mRNA levels decreased with increasing miR-122 concentrations in a dose-dependent manner (FIG. 4D). Although miR-122 concentrations of 1-500 nM were tested, only when the miR-122 mimic concentration was above 100 nM, did T7B3* show significant knockdown of HIF1α.

In order to validate that ASO activation is driven through toehold-mediated strand displacement, a T7B3* with its toehold domain truncated we created. After co-transfection with miR-122 mimic, T7B3* lacking the toehold showed a dampened and not statistically significant knockdown of HIF1α mRNA, in contrast to T7B3* with the toehold (FIG. 4E). This knockdown is likely caused by displacement that is not mediated by the toehold over the 24 h duration of the experiment. In addition, T7B3* were co-transfected with miR-122 mimic or a miR-122 sequence with a scrambled toehold-binding domain (1-7nt from 5′ end). The scr. 1-7nt miR-122 did not trigger significant HIF1α knockdown (FIG. 4F). These results demonstrate that toehold binding facilitates the activation of the conditional EZN2968, both thermodynamically (because of weaker binding between the scr. 1-7nt miR-122 and locking strand) and kinetically (through acceleration of the displacement reaction).

Activation of Conditional EZN2968 is Specific to miR-122

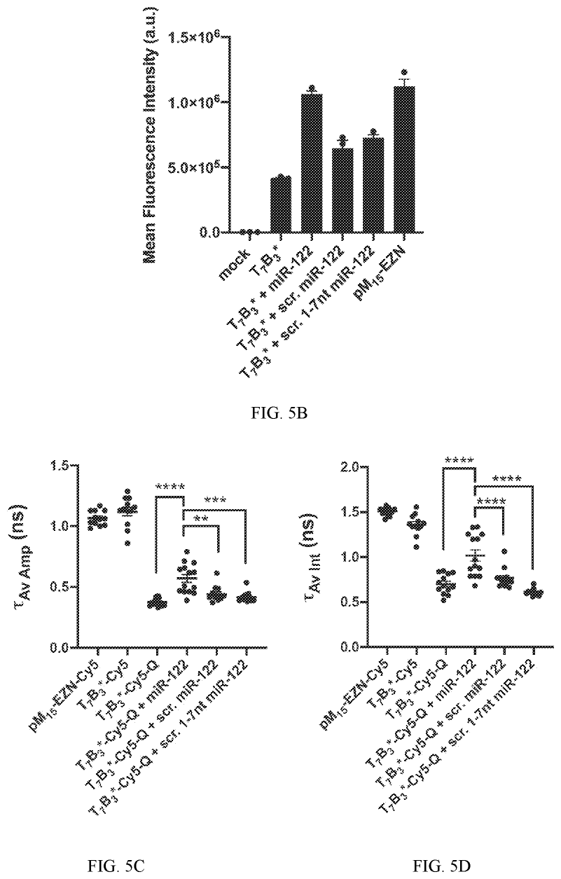

In order to test specificity of conditional EZN2968 activation in response to miR-122 sequence, two scrambled miR-122 sequences were used: one that is completely scrambled, the other with the scrambled toehold binding domain at 1-7 nt of miR-122. Each of these miRNA sequences were co-transfected with conditional ASO T7B3* in U373 cells. The T7B3* duplex was dual labelled with a Cy5 at the 3′ end of the pM15-EZN strand and a quencher on the 5′ end of the B3* strand (FIG. 5A). These labels generate a turn-on fluorescence response upon separation of the conditional ASO and provide a readout of displacement. After 24 h incubation with the miR-122 mimic (500 nM) along with T7B3* (10 nM), the cell-associated fluorescence was measured using flow cytometry. The results showed that the fluorescence of U373 cells showed the largest increase compared to T7B3* alone transfected cells, whereas U373 cells co-transfected with T7B3* and scrambled miR-122 or scrambled 1-7 nt miR-122 showed weaker fluorescence (FIG. 5B). Indeed, the cells co-transfected with T7B3* and miR-122 showed fluorescence levels on par with that of the Cy-5 tagged ASO (pM15-EZN), which confirms miR-122 driven unlocking of the ASO.

To further validate the fluorescence intensity-based assay, the fluorescence lifetime of the Cy5/quencher tagged T7B3* was measured, confirming the specificity of miRNA-inducible ASO activation (FIG. 5A). Fluorescence lifetime measurements provide a concentration-independent readout of the fluorophore local environment and have been used to quantify FRET efficiency of biosensors in cells. In PBS, Cy5 tagged pM15-EZN strands showed an amplitude-averaged lifetime (TAV Amp) of 1.7 ns and an intensity-averaged lifetime (TAV Int) of 1.9 ns; whereas after the pM15-EZN strand was hybridized to locking strand B3* tagged with quencher (Q), TAV Amp and TAV Int of Cy5 decreased to 0.4 ns and 0.7 ns, respectively. After incubation of 10 nM Cy5-Q pair labeled T7B3* with 500 nM miR-122 mimic, TAV Amp and TAV Int increased to 0.9 ns and 1.5 ns. However, the fluorescence lifetime of T7B3* with scrambled miR-122 and scr. 1-7nt miR-122 showed only slight increase compared to T7B3* alone group, which is probably due to non-specific binding of the miRNA mimics to the T7B3*.

Next, U373 cells were co-transfected with T7B3* and miR-122 mimic or scrambled miRNA triggers. After 24 h, fluorescence lifetime imaging microscopy (FLIM) was conducted on the cells to measure Cy5 fluorescence lifetime within cells. pM15-EZN-Cy5 and T7B3* labeled with only Cy5 but not Q were also transfected as positive controls. As shown in FIGS. 5C and 5D, cells co-transfected with T7B3* and miR-122 mimics showed a significant increase of Cy5 fluorescence lifetime compared to the T7B3* only group, whereas cells co-transfected with T7B3* and completely scrambled miR-122 or scrambled 1-7nt miR-122 did not show an increase in fluorescence lifetime. Together, these results confirm that dequenching of Cy5 is caused by separation of the pM15-EZN and locking strand B3* and this response is specifically triggered by miR-122 and mediated by the toehold domain.

Endogenous miR-122 Induces HIF1α Knockdown by Conditional EZN2968

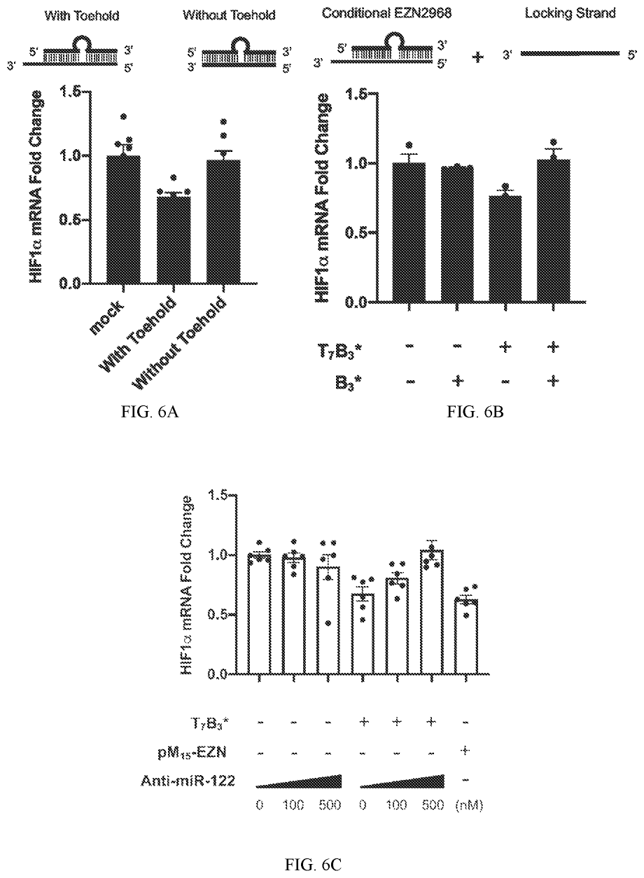

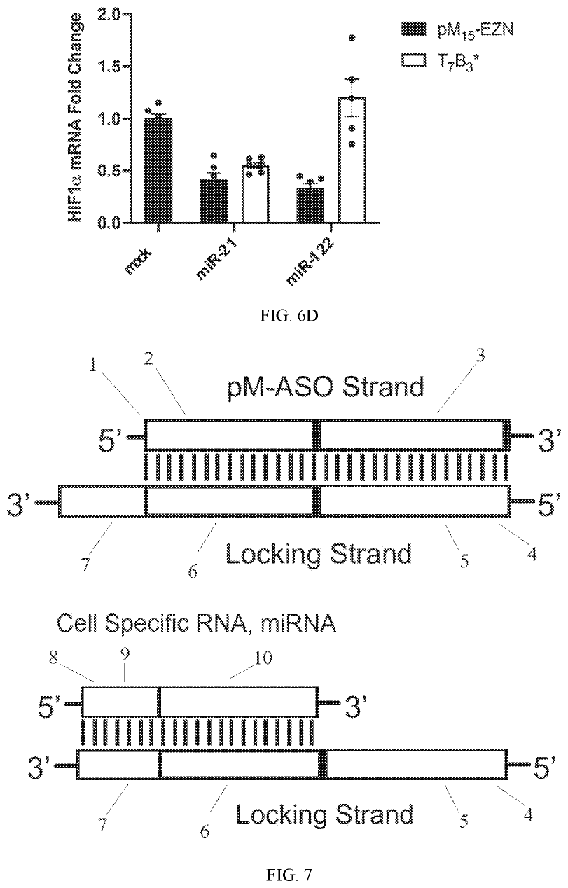

As it is unpredictable whether the miR-122 mimic reproduces the properties of cellular miR-122, experiments were performed to determine whether endogenous miR-122 could induce HIF1α knockdown by T7B3*. T7B3* were transfected into Huh7 cells, a hepatocellular carcinoma cell line that express high levels of miR-122. T7B3* knocked down HIF1α mRNA by ˜40%, whereas T7B3* with the toehold truncated did not show significant knockdown of HIF1α mRNA, suggesting that the activation by endogenous miR-122 proceeds through the intended toehold-mediated strand displacement mechanism (FIG. 6A).

Next, experiments were performed to determine whether sponging of endogenous miR-122 could prevent the activation of T7B3*. T7B3* was co-transfected with excess amounts of locking strand B3* to compete for endogenous miR-122 binding to T7B3*. The result showed that there was no HIF1α knockdown in these co-transfected groups (FIG. 6B). This was further confirmed by miR-122 knockdown experiments in Huh7 cells treated with different concentrations of commercially available anti-miR-122. With increasing concentration of anti-miR-122, the HIF1α knockdown efficacy of T7B3* in Huh7 cells was reduced with complete inhibition of conditional ASO activity at 500 nM anti-miR-122 concentration (FIG. 6C). Together, these data indicate that the conditional EZN2968 activity is induced by endogenous miR-122 and depends on miR-122 expression levels in the cells.

In principle, the conditional EZN2968 design is modular and one could engineer responses to virtually any miRNA trigger. To test the modular design concept, a miR-21-inducible EZN2968 was created with the same toehold length and bulge size as miR-122 inducible T7B3*.

TABLE 3
Oligonucleotide sequences for
miR-21-inducible EZN2968,
“+” = LNA modification;
“*” = PS modification;
/3AmMO/ = 3′ amino modification

| ID | Sequence |
|---|---|
| pMI-EZN | T*C*A* G*A*C* T*G*A* |
| for | T*G*T* T*G*A* +T*+G*+G* |
| miR-21- | C*A*A* G*C*A* T*C*C* |
| inducible | +T*+G*+T* A/3AmMO/ |
| EZN2968 | SEQ ID NO: 2674 |
| B₃* for | /5IAbRQ/*T*A* C*A*G* |
| miR-21- | G*A*T* G*C*+T* +T*+G*+T* |
| inducible | +C*+A*A* C*A*T* C*A*G* |
| EZN2968 | T*C*T* G*A*T* A*A*G* |
| | C*T*A |
| | SEQ ID NO: 2675 |

This duplex was transfected into U373 cells, which express high levels of miR-21. miR-21-inducible T7B3* showed a significant knockdown of HIF1α, while the miR-122-inducible T7B3* did not (FIG. 6D). Thus, by simply changing the miRNA sequence in the extended EZN2968 ASO and the miRNA-complementary sequence in the locking strand, it was possible to generate another conditional EZN2968 ASO triggered by endogenous miR-21. Flow cytometry further showed that cells transfected with fluorophore-quencher labeled miR-21-inducible duplex showed slightly higher fluorescence compared to the miR-122-inducible counterpart, suggesting displacement of the duplex triggered by miR-21. The small differences in fluorescence intensity between miR-21 and miR-122-inducible duplexes may suggest that the down regulation of HIF1α by miR-21-inducible ASO is caused by a small fraction of ASOs that are activated. These results demonstrate the concept of the miRNA-inducible, modular conditional ASO therapeutics, which can be rapidly designed and synthesized based on the disease- or cell-specific miRNAs.

Most ASOs are 16 nts long while most miRNAs are 22 nt, and accordingly, conditional ASOs can be adapted to other specific targets. In principle, conditional ASOs can be engineered to trigger inhibition of any mRNA using other specific miRNA triggers. This concept was further supported by adapting the conditional EZN2968 ASO design to miR-21, showing that miR-21-inducible EZN2968 knocked down HIF1α in cells expressing high levels of miR-21. This miR-21-inducible HIF1α inhibitor could be a potential therapeutic for cancer, given the high expression level and significant roles of miR-21 and HIF1α in cancer development.

What is claimed is:

1. A non-naturally occurring double stranded nucleobase polymer complex comprising
a first strand comprising,
a first domain with a first segment of cell specific RNA and
a second domain that is complementary to a segment of target RNA; and
a second strand comprising,
a first domain that is complementary to the second domain of the first strand,
a second domain that is complementary the first domain of the first strand second, and
a third domain that is complementary to a second segment of the cell specific RNA, wherein the third domain is not complementary to the first strand providing single stranded segment.

2. The double stranded nucleobase polymer complex of claim 1, wherein the first domain of the first strand is on the 5′ end of the first strand.

3. The double stranded nucleobase polymer complex of claim 1, wherein the second domain of the first strand is on the 3′ end of the first strand.

4. The double stranded nucleobase polymer complex of claim 1, wherein the first domain of the second strand is on the 5′ end of the second strand.

5. The double stranded nucleobase polymer complex of claim 1, wherein the second domain of the second strand is on the 3′ end of the first domain of the second strand.

6. The double stranded nucleobase polymer complex of claim 1, wherein the third domain of the second strand is on the 3′-end of the second strand.

7. The double stranded nucleobase polymer complex of claim 1, wherein the cell specific RNA is microRNA.

8. The double stranded nucleobase polymer complex of claim 1, wherein the cell specific RNA is miR-122 or miR-21.

9. The double stranded nucleobase polymer complex of claim 1, wherein the target RNA is messenger RNA.

10. The double stranded nucleobase polymer complex of claim 1, wherein the target RNA is HIF1alpha messenger RNA.

11. The double stranded nucleobase polymer complex of claim 1, wherein between the first domain of the first strand and the second domain of the first strand is one or more nucleobases that do not base pair with the second strand providing a bulge.

12. The double stranded nucleobase polymer complex of claim 11, wherein one or more nucleobases that do not base pair with the second strand are one, two or three nucleobases.

13. The double stranded nucleobase polymer complex of claim 11, wherein the third domain of the second strand is between 12 and 4 nucleobases.

14. The double stranded nucleobase polymer complex of claim 11, wherein the first strand comprises CAATGGTGTTTGTGGCAAGCATCCTGT (SEQ ID NO: 1) (pM12-EZN miR-122), TGACAATGGTGTTTGTGGCAAGCATCCTGT (SEQ ID NO: 2) (pM15-EZN miR-122), or GTGTGACAATGGTGTTTGTGGCAAGCATCCTGT (SEQ ID NO: 3) (pM18-EZN miR-122) wherein each T is optionally U.

15. The double stranded nucleobase polymer complex of claim 11, wherein the second strand comprises
TACAGGATGCTTGCCACAAACACCATTGTCACACTCCA (SEQ ID NO: 13) (B0 miR-122), or
TACAGGATGCTTGCAAACACCATTGTCACACTCCA (SEQ ID NO: 14) (B3 miR-122), wherein each T is optionally U.

16. The double stranded nucleobase polymer complex of claim 11, wherein the first strand comprises TGACAATGGTGTTTGTGGCAAGCATCCTGT (SEQ ID NO: 2) (pM15-EZN miR-122), and the second strand comprises TACAGGATGCTTGCAAACACCATTGTCACACTCCA (SEQ ID NO: 14) (B3 miR-122), wherein each T is optionally U.

17. The double stranded nucleobase polymer complex of claim 16, wherein the first strand is T*G*A*C*A*A*T*G*G*T*G*T*T*T*G*+T*+G*+G*C*A*A*G*C*A*T*C*C*+T*+G*+T* (SEQ ID NO: 2) (pM15-EZN miR-122) and the second strand is *T*A*C*A*G*G*A*T*G*C*+T*+T*+G*+C*+A*+A*A*C*A*C*C*A*T*T*G*T*C*A*C*A*C*T*C*C*A (SEQ ID NO: 14) (B3 miR-122) wherein + is the locked nucleobase and * is a phosphorothioate.

18. The double stranded nucleobase polymer complex of claim 11, wherein the first strand comprises TCAGACTGATGTTGATGGCAAGCATCCTGT (SEQ ID NO: 2674) (pM15-EZN miR-21), and the second strand comprises TACAGGATGCTTGTCAACATCAGTCTGATAAGCTA (SEQ ID NO: 2675) (B3 miR-21), wherein each T is optionally U.

19. The double stranded nucleobase polymer complex of claim 18, wherein the first strand is T*C*A*G*A*C*T*G*A*T*G*T*T*G*A*+T*+G*+G*C*A*A*G*C*A*T*C*C*+T*+G*+T* (SEQ ID NO: 2674) (pM15-EZN miR-21) and the second strand is *T*A*C*A*G*G*A*T*G*C*+T*+T*+G*+T*+C*+A*A*C*A*T*C*A*G*T*C*T*G*A*T*A*A*G*C*T*A (SEQ ID NO: 2675) (B3 miR-21) wherein + is the locked nucleobase and * is a phosphorothioate.

20. A pharmaceutical composition comprising a double stranded nucleobase polymer complex of claim 1 and a pharmaceutically acceptable excipient.

21. A method of treating a disease or condition associated with target RNA overexpression in a cell characterized by cell specific RNA expression comprising administering to a subject in need thereof an effective amount of a double stranded nucleobase polymer complex of claim 1.
